(19) United States
(12) Patent Application Publication
(10) Pub. No.: US 2021/0038585 A1
(43) Pub. Date: Feb. 11, 2021

(54) NIRAPARIB FORMULATIONS

(71) Applicant: Tesaro, Inc., Waltham, MA (US)

(72) Inventors: Simon McGurk, Waltham, MA (US); Padma Narayan, Waltham, MA (US); Clare Medendorp, Waltham, MA (US); George Wu, Waltham, MA (US); Stephen Ruddy, Waltham, MA (US); Heidi Kempinski, Waltham, MA (US); Alistair Stewart, Waltham, MA (US)

(21) Appl. No.: 17/073,198

(22) Filed: Oct. 16, 2020

Related U.S. Application Data
(63) Continuation of application No. 16/584,149, filed on Sep. 26, 2019, now abandoned.
Application No. 16/584,149 is a continuation of application No. PCT/US2018/024597, filed on Mar. 27, 2018.
(60) Provisional application No. 62/477,425, filed on Mar. 27, 2017.

(51) Int. Cl. A61K 31/4439; A61K 9/48; A61K 47/02; A61K 47/12; A61K 47/26

(52) U.S. Cl. CPC A61K 31/4439; A61K 9/4825; A61K 47/26; A61K 47/12; A61K 47/02

(57) ABSTRACT

The present invention relates to pharmaceutical capsule compositions comprising the compound niraparib as an active pharmaceutical ingredient, suitable for oral administration as well as to methods for their preparation. Also described herein are capsule formulations containing niraparib formed by the disclosed methods, and therapeutic uses of such capsule formulations for treating various disorders and conditions. The niraparib is distributed with substantial uniformity throughout a pharmaceutically acceptable carrier of the capsule formulations and exhibit good long-term stability and dissolution properties.

### CROSS REFERENCE

This application claims the benefit of U.S. Provisional Application No. 62/477,425, filed Mar. 27, 2017, which is incorporated herein by reference in its entirety.

### SUMMARY OF THE INVENTION

Niraparib is an orally active and potent poly (ADP-ribose) polymerase, or PARP, inhibitor. Niraparib and pharmaceutically acceptable salts thereof, are disclosed in International Publication No. WO2007/113596 and European Patent No. EP2007733B1; International Publication No. WO2008/084261 and U.S. Pat. No. 8,071,623; and International Publication No. WO2009/087381 and U.S. Pat. No. 8,436,185. Methods of making niraparib and pharmaceutically acceptable salts thereof are disclosed in International Publication Nos. WO2014/088983 and WO2014/088984. Methods to treat cancer with niraparib and pharmaceutically acceptable salts thereof are disclosed in Methods to treat cancer with niraparib and pharmaceutically acceptable salts thereof are disclosed in U.S. Provisional Patent Application Nos. 62/356,461, 62/402,427, 62/470,141, and PCT application PCT/US17/40039. The contents of each of the foregoing references are incorporated herein by reference in their entirety.

PARP is a family of proteins involved in many functions in a cell, including DNA repair, gene expression, cell cycle control, intracellular trafficking and energy metabolism. PARP proteins play key roles in single strand break repair through the base excision repair pathway. PARP inhibitors have shown activity as a monotherapy against tumors with existing DNA repair defects, such as BRCA1 and BRCA2, and as a combination therapy when administered together with anti-cancer agents that induce DNA damage.

Despite several advances in treatment of ovarian cancer, most patients eventually relapse, and subsequent responses to additional treatment are often limited in duration. Women with germline BRCA1 or BRCA2 mutations have an increased risk for developing high grade serous ovarian cancer (HGSOC), and their tumors appear to be particularly sensitive to treatment with a PARP inhibitor. In addition, published scientific literature indicates that patients with platinum sensitive HGSOC who do not have germline BRCA1 or BRCA2 mutations may also experience clinical benefit from treatment with a PARP inhibitor.

It is estimated that 5% to 10% of women who are diagnosed with breast cancer, or more than 15,000 women each year, carry a germline mutation in either their BRCA1 or BRCA2 genes. The development of cancer in these women involves the dysfunction of a key DNA repair pathway known as homologous recombination. While cancer cells can maintain viability despite disruption of the homologous recombination pathway, they become particularly vulnerable to chemotherapy if an alternative DNA repair pathway is disrupted. This is known as synthetic lethality—a situation where the individual loss of either repair pathway is compatible with cell viability; but the simultaneous loss of both pathways results in cancer cell deaths. Since PARP inhibitors block DNA repair, in the context of cancer cells with the BRCA mutation, PARP inhibition results in synthetic lethality. For this reason, patients with germline mutations in a BRCA gene show marked clinical benefit that follows treatment with a PARP inhibitor.

It has surprisingly been found that the solid dosage forms according to the present invention have desirable properties that prevent jamming and/or equipment seizing during encapsulation, prevent adherence of material to encapsulation components and demonstrate suitable content uniformity of dosing units, storage stability, potency, and dissolution profiles.

Provided herein is a method of making a formulation comprising niraparib comprising: obtaining niraparib; obtaining lactose monohydrate that has been screened with a screen; combining the niraparib with the screened lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate.

In some embodiments, obtaining niraparib comprises obtaining niraparib that has been screened. In some embodiments, combining the niraparib with the screened lactose monohydrate comprises combining unscreened niraparib with the screened lactose monohydrate. In some embodiments, combining the niraparib with the screened lactose monohydrate comprises combining screened niraparib with the screened lactose monohydrate.

Provided herein is a method of making a formulation comprising niraparib comprising: obtaining niraparib or obtaining niraparib that has been screened; obtaining lactose monohydrate that has been screened; combining the screened niraparib with the screened lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. In some embodiments, obtaining niraparib that has been screened comprises obtaining niraparib that has been screened with a screen having a mesh size of greater than 425 microns. In some embodiments, obtaining niraparib that has been screened with a screen having a mesh size of greater than about 425 microns comprises obtaining niraparib that has been screened with a screen having a mesh size of about 850 microns or about 1180 microns. In some embodiments, obtaining lactose monohydrate that has been screened with a screen comprises obtaining screened lactose monohydrate that has been screened with a screen having a mesh size of at most about 600 microns. In some embodiments, over about 50% of the screened lactose monohydrate is present as particles with a diameter of between about 53 microns and 500 microns. In some embodiments, the magnesium stearate is magnesium stearate screened with a screen having a mesh size of greater than about 250 microns. In some embodiments, the magnesium stearate is magnesium stearate screened with a screen having a mesh size of about 600 microns. In some embodiments, the method further comprises screening the blended composition comprising niraparib and lactose monohydrate before combining the blended composition comprising niraparib and lactose monohydrate with magnesium stearate. In some embodiments, the blended composition comprising niraparib and lactose monohydrate is screened with a screen having a mesh size of about 600 microns.

Provided herein is a method of making a formulation comprising niraparib comprising: obtaining niraparib that has been screened with a screen having a mesh size of greater than about 425 microns; combining the screened niraparib with lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. In some embodiments, the lactose monohydrate has been screened before combining the screened niraparib with the lactose monohydrate to form a composition comprising niraparib and lactose monohydrate. In some embodiments, the lactose monohydrate that has been screened has been screened with a screen having a mesh size of at most about 600 microns. In some embodiments, over about 50% of the screened lactose monohydrate is present as particles with a diameter of between about 53 microns and 500 microns. In some embodiments, obtaining niraparib that has been screened with a screen having a mesh size of greater than about 425 microns comprises obtaining niraparib that has been screened with a screen having a mesh size of about 850 microns or about 1180 microns. In some embodiments, the magnesium stearate is magnesium stearate screened with a screen having a mesh size of greater than about 250 microns. In some embodiments, the magnesium stearate is magnesium stearate screened with a screen having a mesh size of about 600 microns. In some embodiments, the method further comprises screening the blended composition comprising niraparib and lactose monohydrate before combining the blended composition comprising niraparib and lactose monohydrate with magnesium stearate. In some embodiments, the blended composition comprising niraparib and lactose monohydrate is screened with a screen having a mesh size of about 600 microns.

Provided herein is a method of making a formulation comprising niraparib comprising: obtaining niraparib that has been screened; combining the screened niraparib with lactose monohydrate to form a composition comprising niraparib and lactose monohydrate, blending the composition comprising niraparib and lactose monohydrate, combining the blended composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than about 250 microns, and blending the composition comprising niraparib, lactose monohydrate and magnesium stearate. In some embodiments, the magnesium stearate is magnesium stearate screened with a screen having a mesh size of about 600 microns. In some embodiments, the lactose monohydrate has been screened before combining the screened niraparib with the lactose monohydrate to form a composition comprising niraparib and lactose monohydrate. In some embodiments, the lactose monohydrate has been screened with a screen having a mesh size of at most about 600 microns. In some embodiments, over about 50% of the screened lactose monohydrate is present as particles with a diameter of between about 53 microns and 500 microns. In some embodiments, obtaining niraparib that has been screened comprises obtaining niraparib that has been screened with a screen having a mesh size of greater than about 425 microns. In some embodiments, obtaining niraparib that has been screened with a screen having a mesh size of greater than about 425 microns comprises obtaining niraparib that has been screened with a screen having a mesh size of about 850 microns or about 1180 microns. In some embodiments, the method further comprises screening the blended composition comprising niraparib and lactose monohydrate before combining the blended composition comprising niraparib and lactose monohydrate with magnesium stearate. In some embodiments, the blended composition comprising niraparib and lactose monohydrate is screened with a screen having a mesh size of about 600 microns.

Provided herein is a method of making a formulation comprising niraparib comprising: obtaining niraparib that has been screened; combining the screened niraparib with lactose monohydrate to form a composition comprising niraparib and lactose monohydrate; blending the composition comprising niraparib and lactose monohydrate; screening the blended composition comprising niraparib and lactose monohydrate; combining the screened composition comprising niraparib and lactose monohydrate with magnesium stearate to form a composition comprising niraparib, lactose monohydrate and magnesium stearate; and blending the composition comprising niraparib, lactose monohydrate and magnesium stearate. In some embodiments, the blended composition comprising niraparib and lactose monohydrate is screened with a screen having a mesh size of about 600 microns. In some embodiments, the lactose monohydrate has been screened before combining the screened niraparib with the lactose monohydrate to form a composition comprising niraparib and lactose monohydrate. In some embodiments, the lactose monohydrate has been screened with a screen having a mesh size of at most about 600 microns. In some embodiments, over about 50% of the screened lactose monohydrate is present as particles with a diameter of between about 53 microns and 500 microns. In some embodiments, obtaining niraparib that has been screened comprises obtaining niraparib that has been screened with a screen having a mesh size of greater than about 425 microns. In some embodiments, obtaining niraparib that has been screened with a screen having a mesh size of greater than about 425 microns comprises obtaining niraparib that has been screened with a screen having a mesh size of about 850 microns or about 1180 microns. In some embodiments, the magnesium stearate is magnesium stearate screened with a screen having a mesh size of greater than about 250 microns. In some embodiments, the magnesium stearate is magnesium stearate screened with a screen having a mesh size of about 600 microns. In some embodiments, the screened niraparib has been annealed one or more times.

Provided herein is a method of making a formulation comprising niraparib comprising: obtaining niraparib that has been screened, wherein the niraparib has been annealed two or more times; combining the screened niraparib with lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. In some embodiments, the blended composition comprising niraparib and lactose monohydrate is screened with a screen having a mesh size of about 600 microns. In some embodiments, the lactose monohydrate has been screened before combining the screened niraparib with the lactose monohydrate to form a composition comprising niraparib and lactose monohydrate. In some embodiments, the lactose monohydrate has been screened with a screen having a mesh size of at most about 600 microns. In some embodiments, over about 50% of the screened lactose monohydrate is present as particles with a diameter of between about 53 microns and 500 microns. In some embodiments, obtaining niraparib that has been screened comprises obtaining niraparib that has been screened with a screen having a mesh size of greater than about 425 microns. In some embodiments, obtaining niraparib that has been screened with a screen having a mesh size of greater than about 425 microns comprises obtaining niraparib that has been screened with a screen having a mesh size of about 850 microns or about 1180 microns. In some embodiments, the magnesium stearate is magnesium stearate screened with a screen having a mesh size of greater than about 250 microns. In some embodiments, the magnesium stearate is magnesium stearate screened with a screen having a mesh size of about 600 microns. In some embodiments, the method further comprises screening the blended composition comprising niraparib and lactose monohydrate before combining the blended composition comprising niraparib and lactose monohydrate with magnesium stearate. In some embodiments, the blended composition comprising niraparib and lactose monohydrate is screened with a screen having a mesh size of about 600 microns.

Provided herein is a method of making a formulation comprising niraparib comprising: obtaining niraparib that has been screened with a screen having a mesh size of greater than about 425 microns; obtaining lactose monohydrate that has been screened with a screen; combining the screened niraparib with lactose monohydrate to form a composition comprising niraparib and lactose monohydrate; blending the composition comprising niraparib and lactose monohydrate; screening the blended composition comprising niraparib and lactose monohydrate; combining the screened composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than about 250 microns; and blending the composition comprising niraparib, lactose monohydrate and magnesium stearate.

In some embodiments, the niraparib has been annealed one or two or more times. In some embodiments, the niraparib has been milled. In some embodiments, the niraparib has been wet milled.

In some embodiments, the niraparib is screened with a conical mill, a vibratory sifter, or an oscillating screen. In some embodiments, the niraparib is screened manually or mechanically.

In some embodiments, the method further comprises encapsulating the blended composition comprising niraparib, lactose monohydrate and magnesium stearate into one or more capsules. In some embodiments, the method further comprises encapsulating the formulation comprising niraparib, lactose monohydrate and magnesium stearate into one or more capsules. In some embodiments, the one or more capsules are hard-shelled capsules. In another embodiment, the capsules are soft-shelled capsules. Hard shelled capsules may be gelatin capsules. Hard-shelled capsules are made in two halves: a lower-diameter “body” that is filled and then sealed using a higher-diameter “cap”. Hard capsules may be gelatin capsules. In some embodiments, the encapsulating comprises using an encapsulator. In some embodiments, the encapsulating comprises producing at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 100,000, 150,000, 200,000, 300,000, 400,000, 500,000, or 1 million of the one or more capsules. In some embodiments, the encapsulating comprises producing at a rate of at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 75,000, 100,000, 150,000 or 200,000 of the one or more capsules/hour. In some embodiments, the encapsulating comprises producing the one or more capsules from a batch comprising the composition comprising niraparib, lactose monohydrate and magnesium stearate that is in the encapsulator. In some embodiments, a portion of the volume of the batch in the encapsulator is used to producing the one or more capsules. In some embodiments, the portion of the volume of the batch in the encapsulator used to produce the one or more capsules is less than about 100%, 99%, 98%, 97%, 96%, 95%, 90%, 85%, 80%, or 75% of a total initial volume of the batch. In some embodiments, one or more parts of the encapsulator are coated with a coating. In some embodiments, the one or more coated parts comprises a tamping pin, a dosing disc, or both. In some embodiments, the coating comprises nickel, chrome, or a combination thereof. In some embodiments, the encapsulating comprises automatic encapsulation. In some embodiments, adherence of the composition to one or more coated encapsulating components is reduced or prevented compared to uncoated encapsulating components. In some embodiments, jamming of an encapsulator with coated encapsulating components is reduced or prevented compared to an encapsulator with uncoated encapsulating components.

In some embodiments, blending the composition comprising niraparib and lactose monohydrate comprises blending for about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, or 1000 revolutions. In some embodiments, blending the composition comprising niraparib, lactose monohydrate and magnesium stearate comprises blending for about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, or 1000 revolutions. In some embodiments, the particle size of the lactose monohydrate is the same as the particle size of the niraparib. In some embodiments, the blending comprises using a blender, and wherein the niraparib is distributed with substantial uniformity throughout the blender.

In some embodiments, a dose-to-dose niraparib concentration variation in the one or more capsules is less than about 50%. In some embodiments, the dose-to-dose niraparib concentration variation in the one or more capsules is less than about 40%. In some embodiments, the dose-to-dose niraparib concentration variation in the one or more capsules is less than about 30%. In some embodiments, the dose-to-dose niraparib concentration variation in the one or more capsules is less than about 20%. In some embodiments, the dose-to-dose niraparib concentration variation in the one or more capsules is less than about 10%. In some embodiments, the dose-to-dose niraparib concentration variation in the one or more capsules is less than about 5%. In some embodiments, the dose-to-dose niraparib concentration variation is based on 10 or fewer consecutive doses or capsules. In some embodiments, the dose-to-dose niraparib concentration variation is based on 8 consecutive doses or capsules. In some embodiments, the dose-to-dose niraparib concentration variation is based on 5 consecutive doses or capsules. In some embodiments, the dose-to-dose niraparib concentration variation is based on 3 consecutive doses or capsules. In some embodiments, the dose-to-dose niraparib concentration variation is based on 2 consecutive doses or capsules.

Provided herein is a capsule comprising a formulation comprising an effective amount of niraparib to inhibit polyadenosine diphosphate ribose polymerase (PARP) when administered to a human, lactose monohydrate, and magnesium stearate; wherein the capsule comprises the composition comprising niraparib, lactose monohydrate and magnesium stearate produced according a method described herein. Provided herein is a composition comprising an effective amount of niraparib to inhibit polyadenosine diphosphate ribose polymerase (PARP) when administered to a human, lactose monohydrate, and magnesium stearate; wherein the capsule comprises the composition comprising niraparib, lactose monohydrate and magnesium stearate produced according a method described herein.

Provided herein is a capsule comprising a formulation comprising an effective amount of niraparib to inhibit polyadenosine diphosphate ribose polymerase (PARP) when administered to a human, lactose monohydrate, and magnesium stearate; wherein the niraparib has been annealed two or more times. Provided herein is a capsule comprising a formulation comprising an effective amount of niraparib to inhibit polyadenosine diphosphate ribose polymerase (PARP) when administered to a human, lactose monohydrate, and magnesium stearate; wherein the niraparib has a Hausner's ratio of less than about 1.3 or less than about 1.7 or wherein the niraparib has a Hausner's ratio of less than about 1.3 or less than about 1.8. In some embodiments, the niraparib has a Hausner's ratio of about 1.4 or less. In some embodiments, the niraparib has a Hausner's ratio of about 1.48 or less. In some embodiments, the niraparib has a Hausner's ratio of about 1.38 or less. In some embodiments, the niraparib has a Hausner's ratio of about 1.3-1.7. In some embodiments, the average is about 1.5.

Provided herein is a formulation comprising an effective amount of niraparib to inhibit polyadenosine diphosphate ribose polymerase (PARP) when administered to a human, lactose monohydrate, and magnesium stearate; wherein the niraparib has been annealed two or more times.

Provided herein is a formulation comprising an effective amount of niraparib to inhibit polyadenosine d.iphosphate ribose polymerase (PARP) when administered to a human, lactose monohydrate, and magnesium stearate; wherein the niraparib has a Hausner's ratio of less than about 1.3 or less than about 1.7. In some embodiments, the niraparib has a Hausner's ratio of about 1.48 or less. In some embodiments, the niraparib has a Hausner's ratio of about 1.38 or less. In some embodiments, the niraparib has a Hausner's ratio of about 1.3-1.7 or a range of about 1.4-1.8. In some embodiments, the average can be about 1.5.

Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation in the capsule has a Hausner's ratio of about 1.8 or less. Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation in the capsule has a Hausner's ratio of about 1.63 or less or wherein the formulation on the capsule has a Hausner's ratio in the range of about 1.18-1.63. In some embodiments, the Hausner's ratio is about an average of 1.41.

Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation in the capsule has a Hausner's ratio of about 1.7 or less. In some embodiments, the formulation in the capsule has a Hausner's ratio of about 1.67 or less. In some embodiments, the formulation in the capsule has a Hausner's ratio of about 1.64 or less. In some embodiments, the formulation in the capsule has a Hausner's ratio of about 1.52 or less. In some embodiments, the formulation in the capsule has a Hausner's ratio of about 1.47 or less. In some embodiments, the formulation in the capsule has a Hausner's ratio of about 1.43 or less. In some embodiments, the formulation in the capsule has a Hausner's ratio of about 1.41 or less. In some embodiments, the formulation in the capsule has a Hausner's ratio of about 1.3 or less.

Provided herein is a formulation comprising an effective amount of niraparib to inhibit polyadenosine diphosphate ribose polymerase (PARP) when administered to a human, lactose monohydrate, and magnesium stearate; wherein the has a Hausner's ratio of about 1.7 or less. In some embodiments, the formulation has a Hausner's ratio of about 1.67 or less. In some embodiments, the formulation has a Hausner's ratio of about 1.64 or less. In some embodiments, the formulation has a Hausner's ratio of about 1.52 or less. In some embodiments, the formulation has a Hausner's ratio of about 1.47 or less. In some embodiments, the formulation has a Hausner's ratio of about 1.43 or less. In some embodiments, the formulation has a Hausner's ratio of about 1.41 or less. In some embodiments, the formulation has a Hausner's ratio of about 1.3 or less.

Provided herein is a capsule comprising a formulation comprising an effective amount of niraparib to inhibit polyadenosine diphosphate ribose polymerase (PARP) when administered to a human, lactose monohydrate, and magnesium stearate; wherein the niraparib in the capsule has an internal friction angle of about 29 degrees or higher or about 33.1 degrees or higher.

Provided herein is a formulation comprising an effective amount of niraparib to inhibit polyadenosine diphosphate ribose polymerase (PARP) when administered to a human, lactose monohydrate, and magnesium stearate; wherein the niraparib has an internal friction angle of about 29 degrees or higher or about 33.1 degrees or higher.

Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation in the capsule has an internal friction angle of less than about 34 degrees or of less than about 37 degrees. Provided herein is a formulation comprising an effective amount of niraparib to inhibit polyadenosine diphosphate ribose polymerase (PARP) when administered to a human, lactose monohydrate, and magnesium stearate; wherein the formulation has an internal friction angle of less than about 34 degrees or of less than about 37 degrees.

Provided herein is a capsule comprising a formulation comprising an effective amount of niraparib to inhibit polyadenosine diphosphate ribose polymerase (PARP) when administered to a human, lactose monohydrate, and magnesium stearate; wherein the niraparib has a flow function ratio value of more than about 3.5 or more than about 6.4. Provided herein is a formulation comprising an effective amount of niraparib to inhibit polyadenosine diphosphate ribose polymerase (PARP) when administered to a human, lactose monohydrate, and magnesium stearate; wherein the niraparib has a flow function ratio value of more than about 3.5 or more than about 6.4.

Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation has a flow function ratio value of more than about 6.5 or more than about 14.4. Provided herein is a formulation comprising an effective amount of niraparib to inhibit polyadenosine diphosphate ribose polymerase (PARP) when administered to a human, lactose monohydrate, and magnesium stearate; wherein the formulation has a flow function ratio value of more than about 6.5 or more than about 14.4.

Provided herein is a capsule comprising a formulation comprising an effective amount of niraparib to inhibit polyadenosine diphosphate ribose polymerase (PARP) when administered to a human, lactose monohydrate, and magnesium stearate; wherein the niraparib has a wall friction angle of less than about 29 at an Ra of about 0.05 or of less than about 35 at an Ra of about 0.05. Provided herein is a formulation comprising an effective amount of niraparib to inhibit polyadenosine diphosphate ribose polymerase (PARP) when administered to a human, lactose monohydrate, and magnesium stearate; wherein the niraparib has a wall friction angle of less than about 29 at an Ra of about 0.05 or of less than about 35 at an Ra of about 0.05.

Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 15 degrees at an Ra of about 0.05 or of less than about 25 degrees at an Ra of about 0.05. Provided herein is a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 15 degrees at an Ra of about 0.05 of less than about 25 degrees at an Ra of about 0.05.

Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 26 degrees at an Ra of about 1.2 or of less than about 30 degrees at an Ra of about 1.2. Provided herein is a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 26 degrees at an Ra of about 1.2 or of less than about 30 degrees at an Ra of about 1.2.

Provided herein is a formulation comprising an effective amount of niraparib to inhibit polyadenosine diphosphate ribose polymerase (PARP) when administered to a human, lactose monohydrate, and magnesium stearate; wherein the lactose monohydrate has (i) a bulk density of about 0.2-0.8 mg/cm³ and/or (ii) a tapped density of about 0.3-0.9 mg/cm³. Provided herein is a capsule comprising a formulation comprising an effective amount of niraparib to inhibit polyadenosine diphosphate ribose polymerase (PARP) when administered to a human, lactose monohydrate, and magnesium stearate; wherein the lactose monohydrate has (i) a bulk density of about 0.2-0.8 mg/cm³ and/or (ii) a tapped density of about 0.3-0.9 mg/cm³.

Provided herein is a formulation comprising an effective amount of niraparib to inhibit polyadenosine diphosphate ribose polymerase (PARP) when administered to a human, lactose monohydrate particles, and magnesium stearate; wherein about 50% or more of the lactose monohydrate particles has a diameter of at least about 106 microns, and/or about 50% or more of the lactose monohydrate particles has a diameter of at most about 250 microns. Provided herein is a capsule comprising a formulation comprising an effective amount of niraparib to inhibit polyadenosine diphosphate ribose polymerase (PARP) when administered to a human, lactose monohydrate particles, and magnesium stearate; wherein about 50% or more of the lactose monohydrate particles has a diameter of at least about 106 microns, and/or about 50% or more of the lactose monohydrate particles has a diameter of at most about 250 microns.

In some embodiments, the formulation is stable with respect to niraparib degradation after storage for about 1 month, 3 months, 6 months, 9 months, 12 months, 24 months, or 36 months at 5° C. In some embodiments, the composition comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at 5° C. In some embodiments, the formulation comprises less than about 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, 0.09, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 25° C. and about 60% relative humidity (RH). In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 30° C. and about 65% relative humidity (RH). In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 40° C. and about 75% relative humidity (RH) In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurities after storage for about 1 month, 3 months, 6 months, 9 months, 12 months, 24 months, or 36 months at about 5° C. In some formulation, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurities after storage for about 1 month, 3 months, 6 months, 9 months, 12 months, 24 months, or 36 months at about 25° C. and about 60% relative humidity (RH). In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurities after storage for about 1 month, 3 months, 6 months, 9 months, 12 months, 24 months, or 36 months at about 30° C. and about 65% relative humidity (RH). In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurities after storage for about 1 month, 3 months, 6 months, 9 months, 12 months, 24 months, or 36 months at about 40° C. and about 75% relative humidity (RH). In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 5° C. In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 25° C. and about 60% relative humidity (RH). In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 30° C. and about 65% relative humidity (RH). In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 40° C. and about 75% relative humidity (RH).

In some embodiments, the single unspecified degradation product has a relative retention time of about 1.84. In some embodiments, the single unspecified degradation product has a relative retention time of about 1.93.

In some embodiments, the formulation comprises less than about 3%, 2.5% 2%, 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 5° C. In some embodiments, the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 30° C. and about 65% relative humidity (RH). In some embodiments, the composition comprises less than about 3%, 2.5%, 2.0%, 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 40° C. and 7 about 0% relative humidity (RH).

In some embodiments, the formulation has an absolute bioavailability of niraparib of about 60 to about 90%.

In some embodiments, not less than about 30%, 35%, 40%, 45%, 55%, 60%, 65% 70%, 75%, 80%, 85%, 90%, 95%, or 100% of the niraparib dissolves in about 5, 10, 15, 20, 30, 45, 60, 90, or 120 minutes under dissolution evaluation after storage of the formulation for about 1 month, 3 months, 6 months, 9 months, 12 months, 24 months, or 36 months at about 25° C. and about 60% relative humidity (RH).

In some embodiments, the composition comprises two or more capsules each comprising the formulation. In some embodiments, a formulation comprises niraparib tosylate monohydrate in an amount that is about 19.16%, 38.32%, 57.48%, or 76.64% by weight of the composition.

In some embodiments, a formulation comprises niraparib tosylate monohydrate in an amount that is about 19.2 to about 38.3% w/w niraparib.

In some embodiments, a formulation comprises about 50 mg to about 300 mg of niraparib tosylate monohydrate, about 100 mg to about 200 mg of niraparib tosylate monohydrate, or about 125 mg to about 175 mg of niraparib tosylate monohydrate.

In some embodiments, a formulation comprises about 79.7 mg, about 159.4 mg, about 318.8 mg, or about 478.2 mg niraparib tosylate monohydrate.

In some embodiments, a formulation comprises about 100 mg of niraparib based on free base (e.g., about 159.4 mg niraparib tosylate monohydrate).

In some embodiments, a formulation comprises about 61.2 to about 80.3% w/w lactose monohydrate.

In some embodiments, a formulation comprises at least about 0.5% w/w magnesium stearate.

In embodiments, a capsule comprises any formulation described herein.

Provided herein is a method of treating cancer, comprising administering to a subject in need thereof an effective amount of a formulation or a capsule comprising a formulation as described herein.

In some embodiments, the formulation or capsule is administered in doses having a dose-to-dose niraparib concentration variation of less than 50%, less than 40%, less than 30%, less than 20%, less than 10%, or less than 5%.

In some embodiments, the cancer is selected from the group consisting of ovarian cancer, breast cancer, cervical cancer, endometrial cancer, prostate cancer, testicular cancer, pancreatic cancer, esophageal cancer, head and neck cancer, gastric cancer, bladder cancer, lung cancer, bone cancer, colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma, bladder cancer, liver cancer, kidney cancer, myeloma, lymphoma, and combinations thereof. In some embodiments, the cancer is selected from the group consisting of ovarian cancer, fallopian tube cancer, primary peritoneal cancer, and combinations thereof. In some embodiments, the cancer is a recurrent cancer.

In some embodiments, the subject is a human subject. In some embodiments, the human subject was previously treated with a chemotherapy. In some embodiments, the chemotherapy is a platinum-based chemotherapy. In some embodiments, the human subject had a complete or partial response to the chemotherapy.

In some embodiments, the subject has a mean peak plasma concentration (Cₘₐₓ) of about 600 ng/mL to 1000 ng/mL of the niraparib. In some embodiments, the subject has the mean peak plasma concentration (Cmax) within about 0.5 to 6 hours after the administering. In some embodiments, about 60%, 65%, 70%, 75%, 80%, 85% or 90% of the niraparib is bound to human plasma protein of the subject after the administering. In some embodiments, an apparent volume of distribution (Vd/F) of the niraparib is from about 500 L to about 2000 L after administration to a human subject. In some embodiments, the niraparib has a mean terminal half-life (t_{1/2}) of from about 30 to about 60 hours after the administering. In some embodiments, the niraparib has a mean terminal half-life (t_{1/2}) of from about 32-38 hours after the administering. In some embodiments, the niraparib has a mean terminal half-life (t_{1/2}) of from about 36 hours after the administering. In some embodiments, the niraparib has an apparent total clearance (CL/F) of from about 10 L/hour to about 20 L/hour after the administering. In some embodiments, at least about 60%, 65%, 70%, 75%, 80%, 85%, 90%, 95%, 96%, 97%, 98%, 99%, or 100% of the niraparib is released from the composition within about 1 minute, or within about 5 minutes, or within about 10 minutes, or within about 15 minutes, or within about 30 minutes, or within about 60 minutes or within about 90 minutes after the administering. In some embodiments, the subject has a Cₘᵢₙ niraparib blood plasma level at steady state of from about 10 ng/ml to about 100 ng/ml after the administering. In some embodiments, at least about 70%, 80%, 90%, or 95% of the niraparib is absorbed into the bloodstream of the subject within about 1, 2, 3, 4, 5, 6, 7, 8, 9, 10, 11, 12, 16, 18, or 24 hours after administering.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a schematic of an exemplary manufacturing process of the niraparib capsule.

FIG. 1B is a schematic of an exemplary manufacturing process of the niraparib capsule.

FIG. 2 is an exemplary graph of results of stratified uniformity testing during encapsulation of batch D. It shows the average, minimum, and maximum percent label claim values across the encapsulation process.

FIG. 3 is an exemplary graph of particle size of powder blends of batches E, F, G, J, K, and L.

FIG. 4A is an exemplary diagram of a level of a blend in blender showing an exemplary point where capsule fill may be cutoff in some embodiments.

FIG. 4B is a diagram of an exemplary blender attached to a transfer chute.

FIG. 4C is a diagram of an exemplary transfer chute. The transfer chute can be attached to a blender and a powder blend can be transferred from the blender to an encapsulator through the transfer chute.

FIG. 4D is a diagram of an exemplary transfer chute.

FIG. 5 is an exemplary graph of individual stratified content uniformity data from different batches tested. One capsule (from batch K) tested at 170 minutes resulted in an assay value of 88.3%, but this capsule would have been rejected during weight sorting because it was outside of the in-process range. Stratified content uniformity (SCU) samples are not weight sorted.

FIG. 6 is an exemplary graph of sampling location of the encapsulator dosing bowl for batches E, F, G, J, K, and L.

FIG. 7 is an exemplary illustration of an apparatus used in an USP dissolution evaluation.

FIG. 8 is an exemplary illustration of an apparatus used in an USP dissolution evaluation.

FIG. 9 is an exemplary illustration of an apparatus used in an USP dissolution evaluation.

FIG. 10A depicts an exemplary scanning electron microscope (SEM) image of niraparib particles used in a batch.

FIG. 10B depicts an exemplary scanning electron microscope (SEM) image of niraparib particles used in a batch.

FIG. 10C depicts an exemplary scanning electron microscope (SEM) image of niraparib particles used in a batch.

FIG. 10D depicts an exemplary scanning electron microscope (SEM) image of niraparib particles used in a batch.

FIG. 10E depicts an exemplary scanning electron microscope (SEM) image of niraparib particles used in a batch.

FIG. 10F depicts an exemplary scanning electron microscope (SEM) image of niraparib particles used in a batch

FIG. 10G depicts an exemplary scanning electron microscope (SEM) image of niraparib particles used in a batch.

FIG. 10H depicts an exemplary scanning electron microscope (SEM) image of niraparib particles used in a batch.

FIG. 10I depicts an exemplary scanning electron microscope (SEM) image of niraparib particles used in a batch.

FIG. 11 shows an exemplary X-ray powder diffraction pattern for crystalline Form I of 2-{4-[(3S)-piperidin-3-yl]phenyl}-2H-indazole-7-carboxamide.

### DETAILED DESCRIPTION OF THE INVENTION

Various pharmaceutical products are packaged in the form of capsules for oral dosage and release of a pharmaceutically active composition within an individual's body. Oral dosage pharmaceutical capsules are typically filled with microparticulate material or granules on the order of several microns. The encapsulated particles typically contain a select amount of one or more pharmaceutically active compositions along with one or more inert excipient materials. In a typical encapsulation process, a source of particulate material or particles to be encapsulated is transferred from a blender to an encapsulator, where the encapsulator determines the amount of particles to be added to each capsule. The encapsulator transfers the requisite amount of particles into an open capsule (e.g., an open shell portion of the capsule), and the open capsule is then sealed (e.g., by placing a shell cap over the open shell portion filled with particles).

Depending upon the physical attributes of the particles to be encapsulated for the oral dosage product (e.g., variations in particle size, tackiness of the particulate material, irregularities in particle surface geometries, etc.), problems may occur in the encapsulation process, such as jamming of the encapsulator, for example, due to undesired flow properties of the powder. For example, when the particles to be encapsulated have non-spherical and/or irregular geometric surfaces, the particles may frictionally adhere to each other or the walls of the encapsulator, rather than sliding with respect to each other, as the particles are fed through the encapsulator. Significant and undesirable deviations in the consistency and amount of particles transferred through the encapsulator and, thus, to the pharmaceutical capsules being produced can result. In preparing product capsules with particulate material that has undesirable flow properties for encapsulation, the capsules may, for example, decrease in fill weight during the production process, or segregation may occur. For example, during encapsulation in a batch production process, segregation of the original blend may occur with increasing production time. Described herein is an improved system and method for ensuring consistent and accurate dosage amounts of particulate material in the production of oral dosage pharmaceutical products, particularly niraparib capsule products. Oral dosage pharmaceutical capsules are formed in accordance with the present invention that contain particles of particular geometries and particle size distributions while substantially maintaining the capsule weight and particle size distribution of each capsule within a desired range. Preferably, a majority of the capsules in a production batch do not deviate from a target fill weight by more than about 15%, and the average fill weight of a single capsule in the batch does not deviate from the target fill weight by more than about 10%.

Hence, it is recognized that the flowability of powder may be sensitive to the shape and smoothness of the particles of the powder and the size distribution of particles in the powder.

It is, accordingly, among the objects of the present invention to provide dry powder formulations for use as pharmaceuticals, which formulations have for example, improved flow and/or compressibility characteristics facilitating encapsulation in state-of-the-art, high speed production equipment.

### Definitions

The term “AUC” refers to the area under the time/plasma concentration curve after administration of the pharmaceutical composition. AUC_{0-Infinity} denotes the area under the plasma concentration versus time curve from time 0 to infinity; AUC denotes the area under the plasma concentration versus time curve from time 0 to time t.

“Blood plasma concentration” refers to the concentration of compounds provided herein in the plasma component of blood of a subject

The term “bioequivalent” means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. In practice, two products are considered bioequivalent if the 90% confidence interval of the Cₘₐₓ, AUC, or, optionally, Tₘₐₓ is within the range of 80.00% to 125.00%.

“Bulk density”, as used herein, refers to the ratio of the mass of an untapped powder sample and its volume including the contribution of the interparticulate void volume. Bulk density indicates mass of a powder material that can be filled in per unit volume. For example, granules present in the pharmaceutical composition can have a bulk density more than or equal to 0.2-0.8 g/cm³.

The term “Cₘₐₓ” refers to the maximum concentration of isotretinoin in the blood following administration of the pharmaceutical composition.

The term “cancer” includes both solid tumors and hematological malignancies. Cancers include, but are not limited to, ovarian cancer, breast cancer, cervical cancer, endometrial cancer, prostate cancer, testicular cancer, pancreatic cancer, esophageal cancer, head and neck cancer, gastric cancer, bladder cancer, lung cancer (e.g., adenocarcinoma, NSCLC and SCLC), bone cancer (e.g., osteosarcoma), colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma (e.g., liposarcoma), bladder cancer, liver cancer (e.g., hepatocellular carcinoma), kidney cancer (e.g., renal cell carcinoma), myeloid disorders (e.g., AML, CML, myelodysplastic syndrome and promyelocytic leukemia), and lymphoid disorders (e.g., leukemia, multiple myeloma, mantle cell lymphoma, ALL, CLL, B-cell lymphoma, T-cell lymphoma, Hodgkin's lymphoma, non-Hodgkin's lymphoma, hairy cell lymphoma).

The term “capsule” is intended to encompass any encapsulated shell filled with medicines in powder form. Generally, capsules are made of liquid solutions of gelling agents like as gelatin (animal protein) and plant polysaccharides. These include modified forms of starch and cellulose and other derivatives like carrageenans. Capsule ingredients may be broadly classified as: (1) Gelatin Capsules: Gelatin capsules are made of gelatin manufactured from the collagen of animal skin or bone. Also known as gel caps or gelcaps. In gelatin capsules, other ingredients can also be added for their shape, color and hardness like as plasticizers, sorbitol to decrease or increase the capsule's hardness, preservatives, coloring agents, lubricants and disintegrants; (2) Vegetable capsules: They are made of hypromellose, a polymer formulated from cellulose.

The term “composition”, as in pharmaceutical composition, is intended to encompass a drug product comprising niraparib or its pharmaceutically acceptable salts, esters, solvates, polymorphs, stereoisomers or mixtures thereof, and the other inert ingredient(s) (pharmaceutically acceptable excipients). Such pharmaceutical compositions are synonymous with “formulation” and “dosage form”. Pharmaceutical composition of the invention include, but is not limited to, granules, tablets (single layered tablets, multilayered tablets, mini tablets, bioadhesive tablets, caplets, matrix tablets, tablet within a tablet, mucoadhesive tablets, modified release tablets, orally disintegrating tablets, pulsatile release tablets, timed release tablets, delayed release, controlled release, extended release and sustained release tablets), capsules (hard and soft or liquid filled soft gelatin capsules), pills, troches, sachets, powders, microcapsules, minitablets, tablets in capsules and microspheres, matrix composition and the like. In some embodiments, the pharmaceutical composition refers to capsules.

In some embodiments, the pharmaceutical composition refers to hard gelatin capsules or HPMC based capsules. In some embodiments, the pharmaceutical composition refers to hard gelatin capsules.

By “D₅₀”, it is meant that 50% of the particles are below and 50% of the particles are above a defined measurement. D₅₀ can be used to describe different parameters (volume, length, number, area, etc.). D₅₀ as used herein indicates the volume-weighted median diameter, for example, as measured by a laser/light scattering method or equivalent, wherein 50% of the particles, by volume, have a smaller diameter, while 50% by volume have a larger diameter. The volume weighted D₅₀ also relates to the percentage of weight of the particle under a certain size. For example, a D₅₀ of 500 nm means that 50% of the particulate mass is less than 500 nm in diameter and 50% of the particulate mass is greater than 500 nm in diameter. The particle size can be measured by conventional particle size measuring techniques well known to those skilled in the art. Such techniques include, for example, sedimentation field flow fractionation, photon correlation spectroscopy, light scattering (e.g., with a Microtrac UPA 150), laser diffraction and disc centrifugation. For the purposes of the compositions, formulations and methods described herein, effective particle size is the volume median diameter as determined using laser/light scattering instruments and methods, e.g., a Horiba LA-910, or Horiba LA-950. Similarly, “D₉₀” is the volume-weighted diameter, wherein 90% of the particles, by volume, have a smaller diameter, while 10% by volume have a larger diameter and “D₁₀” is the volume-weighted diameter, wherein 10% of the particles, by volume, have a smaller diameter, while 90% by volume have a larger diameter. It is sometimes useful to express the D₅₀ value after sonication. This low power and short period can break up very loose aggregates which will not typically have a negative impact on the in vivo performance of the composition in a subject.

“Diluents” increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like. Combinations of one or more diluents can also be used.

The terms “effective amount” or “therapeutically effective amount,” as used herein, refer to a sufficient amount of the niraparib being administered that would be expected to relieve to some extent one or more of the symptoms of the disease or condition being treated. For example, the result of administration of niraparib disclosed herein is reduction and/or alleviation of the signs, symptoms, or causes of cancer. For example, an “effective amount” for therapeutic uses is the amount of niraparib, including a formulation as disclosed herein required to provide a decrease or amelioration in disease symptoms without undue adverse side effects. The term “therapeutically effective amount” includes, for example, a prophylactically effective amount. It is understood that an “an effective amount” or a “therapeutically effective amount” varies, in some embodiments, from subject to subject, due to variation in metabolism of the compound administered, age, weight, general condition of the subject, the condition being treated, the severity of the condition being treated, and the judgment of the prescribing physician.

The terms “enhance” or “enhancing” refers to an increase or prolongation of either the potency or duration of a desired effect of niraparib, or a diminution of any adverse symptomatology that is consequent upon the administration of the therapeutic agent. Thus, in regard to enhancing the effect of niraparib disclosed herein, the term “enhancing” refers to the ability to increase or prolong, either in potency or duration, the effect of other therapeutic agents that are used in combination with niraparib disclosed herein. An “enhancing-effective amount,” as used herein, refers to an amount of niraparib or other therapeutic agent which is adequate to enhance the effect of another therapeutic agent or niraparib in a desired system. When used in a patient, amounts effective for this use will depend on the severity and course of the disease, disorder or condition, previous therapy, the patient's health status and response to the drugs, and the judgment of the treating physician.

The term “excipient” means a pharmacologically inactive component such as a diluent, lubricant, surfactant, carrier, or the like. Excipients that are useful in preparing a pharmaceutical composition are generally safe, non-toxic and are acceptable for human pharmaceutical use. Reference to an excipient includes both one and more than one such excipient. Co-processed excipients are also covered under the scope of present invention.

“Filling agents” or “fillers” include compounds such as lactose, lactose monohydrate, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, magnesium stearate, calcium hydroxide, talc, sodium stearyl fumarate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Niraparib” is intended to include to encompass niraparib or its pharmaceutically acceptable salts, esters, solvates, polymorphs, stereoisomers or mixtures thereof.

“Particle size” refers to a measured distribution of particles and is usually expressed as the “volume weighted median” size unless specified otherwise.

“Pharmacodynamics” refers to the factors which determine the biologic response observed relative to the concentration of drug.

“Pharmacokinetics” refers to the factors which determine the attainment and maintenance of the appropriate concentration of drug.

“Ready-to-use” refers to pharmaceutical compositions or medical products that can be used without the needs of further changing, modifying, or optimizing the composition or the product prior to administration, for example through dilution, reconstitution, sterilization, etc.

The term “subject” is used to mean an animal, preferably a mammal, including a human or non-human. The terms patient and subject may be used interchangeably.

A “therapeutically effective amount” or “effective amount” is that amount of a pharmaceutical agent to achieve a pharmacological effect. The term “therapeutically effective amount” includes, for example, a prophylactically effective amount. An “effective amount” of niraparib is an amount needed to achieve a desired pharmacologic effect or therapeutic improvement without undue adverse side effects. The effective amount of a niraparib will be selected by those skilled in the art depending on the particular patient and the disease. It is understood that “an effective amount” or a “therapeutically effective amount” can vary from subject to subject, due to variation in metabolism of niraparib, age, weight, general condition of the subject, the condition being treated, the severity of the condition being treated, and the judgment of the prescribing physician. As used herein, amelioration or lessening of the symptoms of a particular disease, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

The term “tₘₐₓ” refers to the time in hours when Cₘₐₓ is achieved following administration of the pharmaceutical composition.

The terms “treat,” “treating” or “treatment,” as used herein, include alleviating, abating or ameliorating a disease or condition, for example cancer, symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, “weight percent,” “wt %,” “percent by weight,” “% by weight,” and variations thereof refer to the concentration of a substance as the weight of that substance divided by the total weight of the composition and multiplied by 100.

Other objects, features, and advantages of the methods and compositions described herein will become apparent from the following detailed description. It should be understood, however, that the detailed description and the specific examples, while indicating specific embodiments, are given by way of illustration only.

### Niraparib Formulations

The present invention recognizes the need to provide improved dosage forms of niraparib having desirable dissolution profiles, pharmacokinetic characteristics, flow properties, and/or good storage stability. There are multiple challenges associated with formulation, process and stability of niraparib as a final formulation or composition. Considerations are interrelated and resolved with a multi focused effort comprising various manufacturing considerations such as formulation, process and equipment considerations. Niraparib presents manufacturing challenges associated with its cohesive nature, which led to powder flow and segregation challenges. The present invention resolves these challenges and provides improved dosage forms of niraparib having desirable properties.

The present invention relates to a process for the preparation of a solid, orally administrable pharmaceutical composition, comprising a poly (adenosine diphosphate [ADP]-ribose) polymerase (PARP)-1 and -2 inhibitor, and its use for the prophylaxis and/or treatment of diseases. The present invention relates to solid dosage forms of niraparib and pharmaceutically acceptable salts thereof (e.g., niraparib tosylate monohydrate), having desirable pharmacokinetic characteristics which exhibit favorable storage stability and dissolution properties. Niraparib has the following structure:

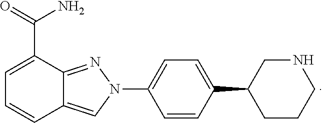 [SMILES: NC(=O)c1cccc2cn(-c3ccc([C@@H]4CCCNC4)cc3)nc12]

Niraparib is an orally available, selective poly(ADP-ribose) polymerase (PARP) 1 and 2 inhibitor. Niraparib displays PARP 1 and 2 inhibition with IC₅₀=3.8 and 2.1 nM, respectively, and in a whole cell assay, it inhibited PARP activity with EC₅₀=4 nM and inhibited proliferation of cancer cells with mutant BRCA-1 and BRCA-2 with CC₅₀ in the 10-100 nM range (see Jones et al., Journal Medicinal Chemistry, 2009, 52, 7170-7185). Methods of administering niraparib to cancer patients are also described in WO2018/005818, which is hereby incorporated by reference in its entirety.

The chemical name for niraparib tosylate monohydrate is 2-{4-[(3S)-piperidin-3-yl]phenyl}-2H-indazole 7-carboxamide 4-methylbenzenesulfonate hydrate (1:1:1) and it has the following chemical structure:

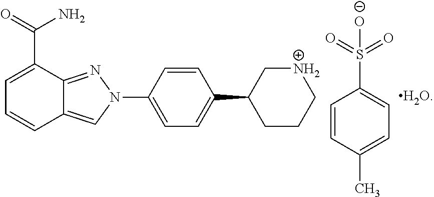

The empirical molecular formula for niraparib is C₂₆H₃₀N₄O₅S and its molecular weight is 510.61 g/mol. Niraparib tosylate monohydrate drug substance is a white to off-white, non-hygroscopic crystalline solid. Niraparib solubility is pH independent below the pKa of 9.95, with an aqueous free base solubility of 0.7 mg/mL to 1.1 mg/mL across the physiological pH range.

Methods for preparation of niraparib include those described in WO 2014/088983; WO 2014/088984; U.S. Pat. Nos. 8,071,623; 8,436,185; U.S. 62/489,415, filed Apr. 24, 2017; and Jones et al., J Med. Chem., 52:7170-7185, 2009, each of which is incorporated by reference in its entirety.

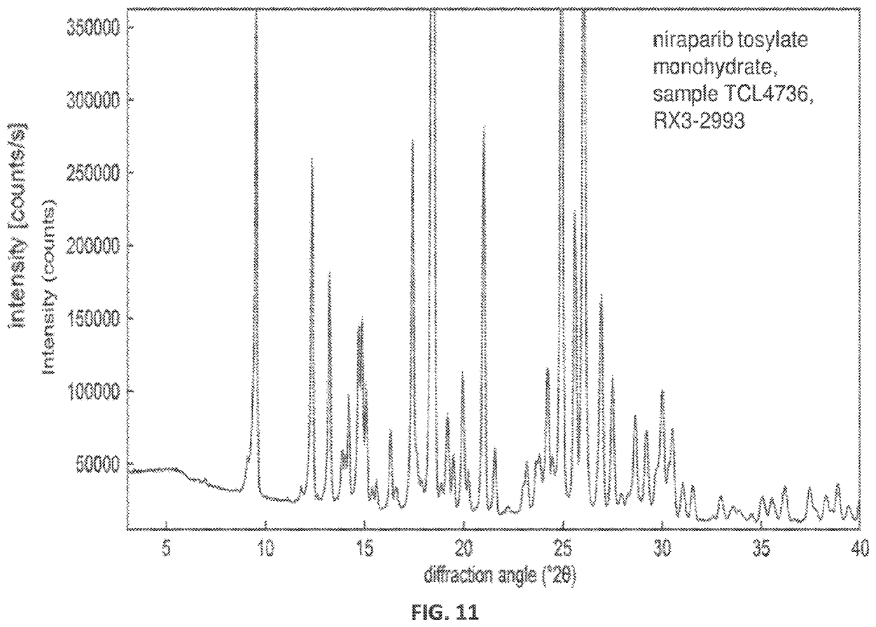

Methods for the preparation of certain solid forms of niraparib are described in U.S. 62/477,411, filed Mar. 27, 2017, which is incorporated by reference in its entirety. In some embodiments, niraparib is provided as crystalline Form I of 2-{4-[(3S)-piperidin-3-yl]phenyl}-2H-indazole-7-carboxamide. Crystalline Form I of 2-{4-[(3S)-piperidin-3-yl]phenyl}-2H-indazole-7-carboxamide is the 4-toluenesulfonate salt and is a monohydrate. In some embodiments, a composition or formulation described herein comprises crystalline Form I of 2-{4-[(3S)-piperidin-3-yl]phenyl}-2H-indazole-7-carboxamide substantially free of Form II and Form III of 2-{4-[(3S)-piperidin-3-yl]phenyl}-2H-indazole-7-carboxamide. Another embodiment provides the composition wherein the crystalline Form I of 2-{4-[(3S)-piperidin-3-yl]phenyl}-2H-indazole-7-carboxamide has an X-ray powder diffraction pattern substantially as shown in FIG. 11. Another embodiment provides the composition where the crystalline Form I of 2-{4-[(3S)-piperidin-3-yl]phenyl}-2H-indazole-7-carboxamide is characterized by at least one X-ray diffraction pattern reflection selected from a 2θ value of 9.5±0.2, 12.4±0.2, 13.2±0.2, 17.4±0.2, 18.4±0.2, 21.0±0.2, 24.9±0.2, 25.6±0.2, 26.0±0.2, and 26.9±0.2.

Niraparib is a selective poly(ADP-ribose) polymerase (PARP) 1 and 2 inhibitor which selectively kills tumor cells in vitro and in mouse xenograft models. PARP inhibition leads to irreparable double strand breaks (DSBs), use of the error-prone DNA repair pathway, resultant genomic instability, and ultimately cell death. Additionally, PARP trapped at genetic lesions as a result of the suppression of autoparlyation can contribute to cytotoxicity.

ZEJULA™ is indicated for the maintenance or treatment of adult patients with recurrent epithelial ovarian, fallopian tube, or primary peritoneal cancer following a complete or partial response to platinum-based chemotherapy. Each ZEJULA™ capsule contains 100 mg of niraparib (as tosylate monohydrate). The hard capsules have a white body with “100 mg” printed in black ink, and a purple cap with “Niraparib” printed in white ink. The recommended dose of ZEJULA™ as monotherapy is three 100 mg capsules taken orally once daily, equivalent to a total daily dose of 300 mg.

Provided herein is an oral composition containing niraparib or its pharmaceutically acceptable salts. In some embodiments, the oral composition includes from about 20 wt % to about 60 wt % of niraparib for treatment of a disorder or condition such as cancer; and a pharmaceutically acceptable carrier, wherein the niraparib is distributed with substantial uniformity throughout the pharmaceutically acceptable carrier.

In some embodiments, the disorder or condition is cancer, for example, ovarian cancer.

In some embodiments, the niraparib can be a pharmaceutically acceptable salt thereof. In some embodiments, the pharmaceutically acceptable salt is niraparib tosylate monohydrate.

In some embodiments, the pharmaceutical composition comprises about 50 mg to about 300 mg of niraparib tosylate monohydrate. For example, the pharmaceutical composition can comprise about 100 mg to about 200 mg of niraparib tosylate monohydrate. For example, the pharmaceutical composition can comprise about 125 mg to about 175 mg of niraparib tosylate monohydrate.

The formulation can comprise one or more components, including niraparib. The components can be combined to create a powder blend that is used to fill capsules. For example, the powder blend can be filled into gelatin capsules, such as size 0 gelatin capsules.

The niraparib may be present in the formulation as a pharmaceutically acceptable salt. For example, the niraparib can be niraparib tosylate monohydrate.

The formulation can comprise one or more diluents. For example, the formulation can comprise lactose monohydrate.

The formulation can comprise one or more lubricants. For example, the formulation can comprise magnesium stearate.

An exemplary niraparib formulation of the present invention comprises 100 mg of niraparib (based on free base, 1.000 mg niraparib anhydrous free base is equivalent to 1.594 mg niraparib tosylate monohydrate), lactose monohydrate and magnesium stearate. An exemplary niraparib formulation of the present invention comprises 100 mg of niraparib (based on free base, 1.000 mg niraparib anhydrous free base is equivalent to 1.594 mg niraparib tosylate monohydrate), lactose monohydrate, magnesium stearate and tartrazine.

### Pharmacodynamics

Niraparib inhibits PARP-1 and PARP-2 enzymes in vitro with IC₅₀ of 3.8 nM (0.82 ng/mL) and 2.1 nM (0.67 ng/mL), respectively. Niraparib inhibits intracellular PARP activity, with an IC₅₀ of 4 nM (1.28 mg/mL) and an IC₉₀ of 50 nM (16 ng/mL). A single dose of 50 mg/kg niraparib in tumor models resulted in >90% PARP inhibition and with daily dosing, tumor regression. At a dose of 50 mg/kg, tumor concentrations of ˜4567 ng/mL were achieved at 6 hours, which exceeds the PARP IC₉₀ and resulted in tumor regression. In this same model, a dose of 75 mg/kg olaparib did not result in tumor regression; tumor regression was achieved when dosing was switched to a 50 mg/kg dose of niraparib.

As used herein, fasted human pharmacokinetic studies include both single dose, fasted, human pharmacokinetic studies and multiple dose, fasted, human pharmacokinetic studies. Multiple dose, fasted, human pharmacokinetic studies are performed in accordance to the FDA Guidance documents and/or analogous EMEA Guidelines. Pharmacokinetic parameters for steady state values may be determined directly from multiple dose, fasted, human pharmacokinetic studies or may be conveniently determined by extrapolation of single dose data using standard methods or industry standard software such as WinNonlin version 5.3 or higher.

In some embodiments, a once daily oral administration of a niraparib composition described herein to a human subject provides a mean peak plasma concentration (Cₘₐₓ) of 600 ng/mL to 1000 ng/mL. For example, a once daily oral administration of a niraparib composition described herein to a human subject can provide a mean peak plasma concentration (Cₘₐₓ) of about 600 ng/mL, 625 ng/mL, 650 ng/mL, 675 ng/mL, 700 ng/mL, 725 ng/mL, 750 ng/mL, 775 ng/mL, 800 ng/mL, 825 ng/mL, 850 ng/mL, 875 ng/mL, 900 ng/mL, 925 ng/mL, 950 ng/mL, 975 ng/mL or 1000 ng/mL. For example, a once daily oral administration of a niraparib composition described herein to a human subject can provide a mean peak plasma concentration (Cₘₐₓ) of about 804 ng/mL.

In some embodiments, a once daily oral administration of a niraparib composition described herein to a human subject provides a mean peak plasma concentration (Cₘₐₓ) in 0.5 to 6 hours. For example, a once daily oral administration of a niraparib composition described herein to a human subject can provide a mean peak plasma concentration (Cₘₐₓ) in about 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, or 6 hours.

In some embodiments, an absolute bioavailability of niraparib provided in a composition described herein is about 60-90%. For example, an absolute bioavailability of niraparib provided in a composition described herein can be about 60%, 65%, 70%, 75%, 80%, 85% or 90%. For example, an absolute bioavailability of niraparib provided in a composition described herein can be about 73%.

In some embodiments, concomitant administration of a high fat meal does not significantly affect the pharmacokinetics of a niraparib composition described herein after administration of a dose described herein. For example, concomitant administration of a high fat meal may not significantly affect the pharmacokinetics of a niraparib composition described herein after administration of an about 50 mg, 100 mg, 150 mg, 200 mg, 250 mg, 300 mg, 350 mg or 400 mg dose of niraparib.

In some embodiments, niraparib is moderately protein bound to human plasma after administration to a human subject. For example, after administration to a human subject about 60%-90% of the niraparib is protein bound to human plasma. For example, after administration to a human subject about 60%, 65%, 70%, 75%, 80%, 85% or 90% of the niraparib is protein bound to human plasma. For example, after administration to a human subject about 83% of the niraparib is protein bound to human plasma.

In some embodiments, an apparent volume of distribution (Vd/F) of niraparib is from about 500 L to about 2000 L after administration to a human subject. For example, an apparent volume of distribution (Vd/F) of niraparib can be about 500 L, 550 L, 600 L, 650 L, 700 L, 750 L, 800 L, 850 L, 900 L, 950 L, 1000 L, 1100 L, 1200 L, 1300 L, 1350 L, 1400 L, 1450 L, 1500 L, 1600 L, 1700 L, 1800 L, 1900 L or 2000 L after administration to a human subject. For example, an apparent volume of distribution (Vd/F) of niraparib can be about 1220 L after administration to a human subject. For example, an apparent volume of distribution (Vd/F) of niraparib can be about 1074 L after administration to a human subject with cancer.

In some embodiments, following administration of niraparib provided in a composition described herein, the mean terminal half-life (t_{1/2}) of niraparib is from about 40 to 60 hours. For example, following administration of niraparib provided in a composition described herein, the mean terminal half-life (t_{1/2}) of niraparib can be about 40 hours, 42 hours, 44 hours, 46 hours, 48 hours, 50 hours, 52 hours, 54 hours, 56 hours, 58 hours or 60 hours. For example, following administration of niraparib provided in a composition described herein, the mean terminal half-life (t_{1/2}) of niraparib can be about 48 to 51 hours. For example, following administration of niraparib provided in a composition described herein, the mean terminal half-life (t_{1/2}) of niraparib can be about 48 hours, 49 hours, 50 hours or 51 hours.

In some embodiments, following administration of niraparib provided in a composition described herein, the apparent total clearance (CL/F) of niraparib is from about 10 L/hour to about 20 L/hour. For example, following administration of niraparib provided in a composition described herein, the apparent total clearance (CL/F) of niraparib can be about 10 L/hour, 11 L/hour, 12 L/hour, 13 L/hour, 14 L/hour, 15 L/hour, 16 L/hour, 17 L/hour, 18 L/hour, 19 L/hour or 20 L/hour. For example, following administration of niraparib provided in a composition described herein, the apparent total clearance (CL/F) of niraparib can be about 16.2 L/hour.

In some embodiments, the formulations disclosed herein provide a release of niraparib from the composition within about 1 minute, or within about 5 minutes, or within about 10 minutes, or within about 15 minutes, or within about 30 minutes, or within about 60 minutes or within about 90 minutes. In other embodiments, a therapeutically effective amount of niraparib is released from the composition within about 1 minute, or within about 5 minutes, or within about 10 minutes, or within about 15 minutes, or within about 30 minutes, or within about 60 minutes or within about 90 minutes. In some embodiments the composition comprises a niraparib capsule formulation providing immediate release of niraparib. In some embodiments the composition comprises a niraparib capsule formulation providing immediate release of niraparib within about 1 minute, or within about 5 minutes, or within about 10 minutes, or within about 15 minutes, or within about 30 minutes, or within about 60 minutes or within about 90 minutes.

The niraparib formulations and dosage forms described herein display pharmacokinetic profiles that can result in Cₘᵢₙ niraparib blood plasma levels at steady state from about 10 ng/ml to about 100 ng/ml. In one embodiment, the niraparib formulations described herein provide blood plasma levels immediately prior to the next dose (Cₘᵢₙ) at steady state from about 25 ng/ml to about 100 ng/ml. In another embodiment, the niraparib formulations described herein provide Cₘᵢₙ blood plasma levels at steady state from about 40 ng/ml to about 75 ng/ml. In yet another embodiment, the niraparib formulations described herein provide Cₘᵢₙ blood plasma levels at steady state of about 50 ng/ml.

The niraparib formulations described herein are administered and dosed in accordance with good medical practice, taking into account the clinical condition of the individual patient, the site and method of administration, scheduling of administration, and other factors known to medical practitioners. In human therapy, the dosage forms described herein deliver niraparib formulations that maintain a therapeutically effective amount of niraparib of at least 10 ng/ml or typically at least about 100 ng/ml in plasma at steady state while reducing the side effects associated with an elevated Cₘₐₓ blood plasma level of niraparib.

In some embodiments, greater than about 95%; or greater than about 90%; or greater than about 80%; or greater than about 70% of the niraparib dosed by weight is absorbed into the bloodstream within 1, 2, 3, 4, 5, 6, 7, 8, 9, 10, 11, 12, 16, 18, or 24 hours after administration.

### Niraparib Concentration/Amount

By means of methods and compositions described herein, formulations can be made that achieve the desired dissolution characteristics and target pharmacokinetic profiles described herein. For example, therapeutically effective doses of niraparib can be administered once, twice or three times daily in capsules using the manufacturing methods and compositions that have been described herein to achieve these results. In some embodiments, the niraparib or a pharmaceutically acceptable prodrug or salt thereof is present in an amount of from about 20-80 wt %, 45-70 wt %, 40-50 wt %, 45-55 wt %, 50-60 wt %, 55-65 wt %, 60-70 wt %, 65-75 wt %, 70-80 wt %, or 40-60 wt %.

In some embodiments, the compositions described herein have a concentration of niraparib or a pharmaceutically acceptable prodrug or salt thereof of from about 1% to about 50%, from about 5% to about 50%, from about 10% to about 50%, from about 15% to about 50%, from about 20% to about 50%, from about 25% to about 50%, from about 30% to about 50%, from about 35% to about 50%, from about 40% to about 50%, or from about 45% to about 50% by weight of the composition.

In some embodiments, the compositions described herein have a concentration of niraparib or a pharmaceutically acceptable prodrug or salt thereof of from about 1% to about 45%, from about 5% to about 45%, from about 10% to about 45%, from about 15% to about 45%, from about 20% to about 45%, from about 25% to about 45%, from about 30% to about 45%, from about 35% to about 45%, or from about 40% to about 45% by weight of the composition.

In some embodiments, the compositions described herein have a concentration of niraparib or a pharmaceutically acceptable prodrug or salt thereof of from about 1% to about 40%, from about 5% to about 40%, from about 10% to about 40%, from about 15% to about 40%, from about 20% to about 40%, from about 25% to about 40%, from about 30% to about 40%, from about 35% to about 40% by weight of the composition.

In some embodiments, the compositions described herein have a concentration of niraparib or a pharmaceutically acceptable prodrug or salt thereof of from about 1% to about 35%, from about 5% to about 35%, from about 10% to about 35%, from about 15% to about 35%, from about 20% to about 35%, from about 25% to about 35%, or from about 30% to about 35% by weight of the composition.

In some embodiments, the compositions described herein have a concentration of niraparib or a pharmaceutically acceptable prodrug or salt thereof of about 1%, 5%, 10%, 15%, 20%, 25%, 30%, 35%, 40%, 45%, or 50% by weight of the composition. In some embodiments, the compositions described herein have a concentration of niraparib tosylate monohydrate of about 19.16% by weight of the composition. In some embodiments, the compositions described herein have a concentration of niraparib tosylate monohydrate of about 38.32% by weight of the composition. In some embodiments, the compositions described herein have a concentration of niraparib tosylate monohydrate of about 57.48% by weight of the composition. In some embodiments, the compositions described herein have a concentration of niraparib tosylate monohydrate of about 76.64% by weight of the composition.

In some embodiments, the compositions described herein have an amount of niraparib or a pharmaceutically acceptable prodrug or salt thereof of from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, the compositions described herein can have an amount of niraparib tosylate monohydrate of from about 1 mg to about 1000 mg, for example, from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg.

In some embodiments, the compositions described herein have an amount of niraparib or a pharmaceutically acceptable prodrug or salt thereof of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg. For example, the compositions described herein can have an amount of niraparib tosylate monohydrate of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

In some embodiments, the compositions described herein have an amount of niraparib or a pharmaceutically acceptable prodrug or salt thereof of about 25 mg, about 50 mg, about 100 mg, about 150 mg, about 200 mg, about 250 mg, about 300 mg, about 350 mg, about 400 mg, about 450 mg, or about 500 mg. For example, the compositions described herein can have an amount of niraparib tosylate monohydrate of about 25 mg, about 50 mg, about 100 mg, about 150 mg, about 200 mg, about 250 mg, about 300 mg, about 350 mg, about 400 mg, about 450 mg, or about 500 mg. In some embodiments, the compositions described herein have an amount of niraparib tosylate monohydrate of about 79.7 mg. In some embodiments, the compositions described herein have an amount of niraparib tosylate monohydrate of about 159.4 mg. In some embodiments, the compositions described herein have an amount of niraparib tosylate monohydrate of about 318.8 mg. In some embodiments, the compositions described herein have an amount of niraparib tosylate monohydrate of about 478.2 mg.

### Pharmaceutically Acceptable Salts

In some embodiments, the niraparib used in a composition disclosed herein is the form of a free base, pharmaceutically acceptable salt, prodrug, analog or complex. In some instances, the niraparib comprises the form of a pharmaceutically acceptable salt. In some embodiments, with respect to niraparib in a composition, a pharmaceutically acceptable salt includes, but is not limited to, 4-methylbenzenesulfonate salts, sulfate salts, benzenesulfate salts, fumarate salts, succinate salts, and stereoisomers or tautomers thereof. In some embodiments, with respect to niraparib in a composition, a pharmaceutically acceptable salt includes, but is not limited to, tosylate salts. In some embodiments, with respect to niraparib in a composition, a pharmaceutically acceptable salt includes, but is not limited to, tosylate monohydrate salts. In some embodiments, the crystalline form of niraparib tosylate is a hydrate. In some embodiments, the crystalline form of niraparib tosylate is niraparib tosylate monohydrate.

### Capsules

The term capsule is intended to encompass any encapsulated shell filled with medicines in powder form. Generally, capsules are made of liquid solutions of gelling agents like as gelatin (animal protein) and plant polysaccharides. These include modified forms of starch and cellulose and other derivatives like carrageenans. Capsule ingredients may be broadly classified as: (1) Gelatin Capsules: Gelatin capsules are made of gelatin manufactured from the collagen of animal skin or bone. Gelatin capsules are also known as gel caps or gelcaps. In gelatin capsules, other ingredients can also be added for their shape, color and hardness such as plasticizers, sorbitol to decrease or increase the capsule's hardness, preservatives, coloring agents, lubricants and disintegrants; (2) Vegetable capsules: They are made of hypromellose, a polymer formulated from cellulose.

### Pharmaceutically Acceptable Excipients

In some aspects, the pharmaceutical composition disclosed herein comprises one or more pharmaceutically acceptable excipients. Exemplary pharmaceutically acceptable excipients for the purposes of pharmaceutical compositions disclosed herein include, but are not limited to, binders, disintegrants, superdisintegrants, lubricants, diluents, fillers, flavors, glidants, sorbents, solubilizers, chelating agents, emulsifiers, thickening agents, dispersants, stabilizers, suspending agents, adsorbents, granulating agents, preservatives, buffers, coloring agents and sweeteners or combinations thereof. Examples of binders include microcrystalline cellulose, hydroxypropyl methylcellulose, carboxyvinyl polymer, polyvinylpyrrolidone, polyvinylpolypyrrolidone, carboxymethylcellulose calcium, carboxymethylcellulose sodium, ceratonia, chitosan, cottonseed oil, dextrates, dextrin, ethylcellulose, gelatin, glucose, glyceryl behenate, galactomannan polysaccharide, hydroxyethyl cellulose, hydroxyethylmethyl cellulose, hydroxypropyl cellulose, hypromellose, inulin, lactose, magnesium aluminum silicate, maltodextrin, methylcellulose, poloxamer, polycarbophil, polydextrose, polyethylene glycol, polyethylene oxide, polymethacrylates, sodium alginate, sorbitol, starch, sucrose, sunflower oil, vegetable oil, tocofersolan, zein, or combinations thereof. Examples of disintegrants include hydroxypropyl methylcellulose (HPMC), low substituted hydroxypropyl cellulose (L-HPC), croscarmellose sodium, sodium starch glycolate, lactose, magnesium aluminum silicate, methylcellulose, polacrilin potassium, sodium alginate, starch, or combinations thereof. Examples of a lubricant include stearic acid, sodium stearyl fumarate, glyceryl behenate, calcium stearate, glycerin monostearate, glyceryl palmitostearate, magnesium lauryl sulfate, mineral oil, palmitic acid, myristic acid, poloxamer, polyethylene glycol, sodium benzoate, sodium chloride, sodium lauryl sulfate, talc, zinc stearate, potassium benzoate, magnesium stearate or combinations thereof. Examples of diluents include talc, ammonium alginate, calcium carbonate, calcium lactate, calcium phosphate, calcium silicate, calcium sulfate, cellulose, cellulose acetate, corn starch, dextrates, dextrin, dextrose, erythritol, ethylcellulose, fructose, fumaric acid, glyceryl palmitostearate, isomalt, kaolin, lactitol, lactose, magnesium carbonate, magnesium oxide, maltodextrin, maltose, mannitol, microcrystalline cellulose, polydextrose, polymethacrylates, simethicone, sodium alginate, sodium chloride, sorbitol, starch, sucrose, sulfobutylether β-cyclodextrin, tragacanth, trehalose, xylitol, or combinations thereof. In some embodiments, the pharmaceutically acceptable excipient is hydroxypropyl methylcellulose (HPMC). In some embodiments, the pharmaceutically acceptable excipient is low substituted hydroxypropyl cellulose (L-HPC). In some embodiments, the pharmaceutically acceptable excipient is lactose. In some embodiments, the pharmaceutically acceptable excipient is lactose monohydrate. In some embodiments, the pharmaceutically acceptable excipient is magnesium stearate. In some embodiments, the pharmaceutically acceptable excipient is lactose monohydrate and magnesium stearate.

Various useful fillers or diluents include, but are not limited to calcium carbonate (Barcroft™, MagGran™, Millicarb™, Pharma-Carb™, Precarb™, Sturcal™, Vivapres Ca™) calcium phosphate, dibasic anhydrous (Emcompress Anhydrous™, Fujicalin™), calcium phosphate, dibasic dihydrate (Calstar™, Di-Cafos™, Emcompress™), calcium phosphate tribasic (Tri-Cafos™, TRI-TAB™), calcium sulphate (Destab™, Drierite™, Snow White™, Cal-Tab™, Compactrol™) cellulose powdered (Arbocel™, Elcema™, Sanacet™), silicified microcrystailine cellulose, cellulose acetate, compressible sugar (Di-Pac™), confectioner's sugar, dextrates (Candex™, Emdex™) dextrin (Avedex™, Caloreen™, Primogran W™), dextrose (Caridex™, Dextrofin™, Tab fine D-IOO™), fructose (Fructofin™, Krystar™), kaolin (Lion™, Sim 90™), lactitol (Finlac DC™, Finlac MCX™), lactose (Anhydrox™, CapsuLac™, Fast-Flo™, FlowLac™, GranuLac™, InhaLac™, Lactochem™, Lactohaie™, Lactopress™, Microfme™, Microtose™, Pharmatose™, Prisma Lac™, Respitose™, SacheLac™, SorboLac™, Super-Tab™, Tablettose™, Wyndale™, Zeparox™), lactose monohydrate, magnesium carbonate, magnesium oxide (MagGran MO™), maltodextrin (C*Dry MD™, Lycatab DSH™, Maldex™, Maitagran™, Maltrin™, Maltrin QD™, Paselli MD 10 PH™, Star-Dri™), maltose (Advantose 100™), mannitol (Mannogem™, Pearlitol™), microcrystalline cellulose (Avicel PH™, Celex™, Celphere™, Ceolus KG™, Emcocel™, Pharmacel™, Tabulose™, Vivapur™), polydextrose (Litesse™), simethicone (Dow Corning Q7-2243 LVA™, Cow Corning Q7-2587™, Sentry Simethicone™), sodium alginate (Keltone™, Protanal™), sodium chloride (Alberger™), sorbitol (Liponec 70-NC™, Liponic 76-NCv, Meritol™, Neosorb™, Sorbitol Instant™, Sorbogem™), starch (Flufiex W™, Instant Pure-Cote™, Melojei™, Meritena Paygel 55™, Perfectamyl D6PH™, Pure-Cote™, Pure-Dent™, Pure-Ge™, Pure-Set™, Purity 21™, Purity 826™, Tablet White™), pregelatinized starch, sucrose, trehalose and xylitol, or mixtures thereof.

In some embodiments, a filler such as lactose monohydrate is present in an amount of about 5-90% by weight. In some embodiments, a filler such as lactose monohydrate is present in an amount of about 5-80% by weight. In some embodiments, a filler such as lactose monohydrate is present in an amount of about 5-70% by weight. In some embodiments, a filler such as lactose monohydrate is present in an amount of about 5-60% by weight. In some embodiments, a filler such as lactose monohydrate is present in an amount of about 5-50% by weight. In some embodiments, a filler such as lactose monohydrate is present in an amount of about 5-40% by weight. In some embodiments, a filler such as lactose monohydrate is present in an amount of about 5-30% by weight. In some embodiments, a filler such as lactose monohydrate is present in an amount of about 25-90% by weight. In some embodiments, a filler such as lactose monohydrate is present in an amount of about 25-80% by weight. In some embodiments, a filler such as lactose monohydrate is present in an amount of about 25-70% by weight. In some embodiments, a filler such as lactose monohydrate is present in an amount of about 25-60% by weight. In some embodiments, a filler such as lactose monohydrate is present in an amount of about 25-50% by weight. In some embodiments, a filler such as lactose monohydrate is present in an amount of about 25-40% by weight. In some embodiments, a filler such as lactose monohydrate is present in an amount of about 40-90% by weight. In some embodiments, a filler such as lactose monohydrate is present in an amount of about 40-80% by weight. In some embodiments, a filler such as lactose monohydrate is present in an amount of about 40-70% by weight. In some embodiments, a filler such as lactose monohydrate is present in an amount of about 40-60% by weight. In some embodiments, a filler such as lactose monohydrate is present in an amount of about 40-50% by weight. In some embodiments, a filler such as lactose monohydrate is present in an amount of about 40% by weight. In some embodiments, a filler such as lactose monohydrate is present in an amount of about 50% by weight. In some embodiments, a filler such as lactose monohydrate is present in an amount of about 60% by weight. In some embodiments, a filler such as lactose monohydrate is present in an amount of about 70% by weight. In some embodiments, a filler such as lactose monohydrate is present in an amount of about 80% by weight.

In some embodiments, a filler such as lactose monohydrate is present in an amount of from about 25 mg to about 1000 mg, from about 50 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, or from about 500 mg to about 1000 mg. For example, a filler such as lactose monohydrate can be present in an amount of from about 25 mg to about 1000 mg, from about 50 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, or from about 500 mg to about 1000 mg.

In some embodiments, a filler such as lactose monohydrate is present in an amount of from about 25 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, or from about 500 mg to about 550 mg. For example, a filler such as lactose monohydrate can be present in an amount of from about 25 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, or from about 500 mg to about 550 mg.

In some embodiments, a filler such as lactose monohydrate is present in an amount of about 15 mg, about 25 mg, about 50 mg, about 100 mg, about 150 mg, about 200 mg, about 250 mg, about 300 mg, about 350 mg, about 400 mg, about 450 mg, or about 500 mg. For example, a filler such as lactose monohydrate can be present in an amount of about 15 mg, about 25 mg, about 50 mg, about 100 mg, about 150 mg, about 200 mg, about 250 mg, about 300 mg, about 350 mg, about 400 mg, about 450 mg, or about 500 mg. In some embodiments, a filler such as lactose monohydrate is present in an amount of about 334.2 mg. In some embodiments, a filler such as lactose monohydrate is present in an amount of about 254.5 mg. In some embodiments, a filler such as lactose monohydrate is present in an amount of about 174.8 mg. In some embodiments, a filler such as lactose monohydrate is present in an amount of about 95.1 mg. In some embodiments, a filler such as lactose monohydrate is present in an amount of about 15.4 mg.

Various useful disintegrants include, but are not limited to, alginic acid (Protacid™, Satialgine H8™), calcium phosphate, tribasic (TRI-TAB™), carboxymethylcellulose calcium (ECG 505™), carboxymethylcellulose sodium (Akucell™, Finnfix™, Nymcel Tylose CB™), colloidal silicon dioxide (Aerosil™, Cab-O-Sil™, Wacker HDK™), croscarmellose sodium (Ac-Di-Sol™, Pharmacel XL™, Primellose™, Solutab™, Vivasol™), crospovidone (Collison CL™, Collison CL-M™, Polyplasdone XL™), docusate sodium, guar gum (Meyprodor™, Meyprofm™, Meyproguar™), low substituted hydroxypropyl cellulose, magnesium aluminum silicate (Magnabite™, Neusilin™, Pharmsorb™, Veegum™), methylcellulose (Methocel™, Metolose™) microcrystalline cellulose (Avicel PH™, Ceoius KG™, Emcoel™, Ethispheres™, Fibrocel™, Pharmacel™, Vivapur™), povidone (Collison™, Plasdone™) sodium alginate (Kelcosol™, Ketone™, Protanal™), sodium starch glycolate, polacrilin potassium (Amberlite IRP88™), silicified microcrystalline cellulose (ProSotv™), starch (Aytex P™, Fluftex W™, Melojel™, Meritena™, Paygel 55™, Perfectamyl D6PH™, Pure-Bind™, Pure-Cote™, Pure-Dent™, Purity 21™, Purity 826™, Tablet White™) or pre-gelatinized starch (Lycatab PGS™, Merigel™, National 78-1551™, Pharma-Gel™, Prejel™, Sepistab ST 200™, Spress B820™, Starch 1500 G™, Tablitz™, Unipure LD™), or mixtures thereof. In some embodiments, a disintegrant is optionally used in an amount of about 0-10% by weight. In some embodiments, a disintegrant is present in an amount of from about 0.1 mg to 0.5 mg, 0.5 mg to 1 mg, 1 mg to 2 mg, 2 mg to 2.5 mg, 2.5 mg to 5 mg, 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23.5 mg, 23 mg to 25.5 mg, 25 mg to 27.5 mg, 27 mg to 30 mg, 29 mg to 31.5 mg, 31 mg to 33.5 mg, 33 mg to 35.5 mg, 35 mg to 37.5 mg, 37 mg to 40 mg, 40 mg to 45 mg, 45 mg to 50 mg, 50 mg to 55 mg, 55 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg. In some embodiments, a disintegrant is present in an amount of about 0.1 mg, 0.5 mg, 1 mg, 2 mg, 2.5 mg, 5 mg, 7 mg, 9 mg, 11 mg, 13 mg, 15 mg, 17 mg, 19 mg, 21 mg, 23 mg, 25 mg, 27.5 mg, 30 mg, 31.5 mg, 33.5 mg, 35.5 mg, 37.5 mg, 40 mg, 45 mg, 50 mg, 55 mg, 60 mg, 65 mg, 70 mg, 75 mg, 80 mg, 85 mg, 90 mg, 95 mg, or 100 mg.

Various useful lubricants include, but are not limited to, calcium stearate (HyQual™) glycerine monostearate (Imwitor™ 191 and 900, Kessco GMS™, 450 and 600, Myvaplex 600P™, Myvatex™, Rita GMS™, Stepan GMS™, Tegin™, Tegin™ 503 and 515, Tegin 4100™, Tegin M™, Unimate GMS™), glyceryl behenate (Compritol 888 ATO™), glyceryl palmitostearate (Precirol ATO 5™), hydrogenated castor oil (Castorwax MP 80™, Croduret™, Cutina HR™, Fancol™, Simulsol 1293™), hydrogenated vegetable oil 0 type I (Sterotex™, Dynasan P60™, Hydrocote™, Lipovol HS-K™, Sterotex HM™), magnesium lauryl sulphate, magnesium stearate, medium-chain triglycerides (Captex 300™, Labrafac CC™, Miglyol 810™, Neobee M5™, Nesatol™, Waglinol 3/9280™), poloxamer (Pluronic™, Synperonic™), polyethylene 5 glycol (Carbowax Sentry™, Lipo™, Lipoxol™, Lutrol E™, Pluriol E™), sodium benzoate (Antimol™) sodium chloride, sodium lauryl sulphate (Elfan 240™, Texapon Kl 2P™), sodium stearyl fumarate (Pruv™), stearic acid (Hystrene™, Industrene™, Kortacid 1895™, Pristerene™), talc (Altaic™, Luzenac™, Luzenac Pharma™, Magsil Osmanthus™, 0 Magsil Star™, Superiore™), sucrose stearate (Surfhope SE Pharma D-1803 F™) and zinc stearate (HyQual™) or mixtures thereof. Examples of suitable lubricants include, but are not limited to, magnesium stearate, calcium stearate, zinc stearate, stearic acid, talc, glyceryl behenate, polyethylene glycol, polyethylene oxide polymers, sodium lauryl sulfate, magnesium lauryl sulfate, sodium oleate, sodium stearyl fumarate, DL-leucine, colloidal silica, and others as known in the art. In some embodiments a lubricant is magnesium stearate.

In some embodiments, a lubricant such as magnesium stearate is present in an amount of about 0.1-5% by weight. In some embodiments, a lubricant such as magnesium stearate is present in an amount of about 0.1-2% by weight. In some embodiments, a lubricant such as magnesium stearate is present in an amount of about 0.1-1% by weight. In some embodiments, a lubricant such as magnesium stearate is present in an amount of about 0.1-0.75% by weight. In some embodiments, a lubricant such as magnesium stearate is present in an amount of about 0.1-5% by weight. In some embodiments, a lubricant such as magnesium stearate is present in an amount of about 0.2-5% by weight. In some embodiments, a lubricant such as magnesium stearate is present in an amount of about 0.2-2% by weight. In some embodiments, a lubricant such as magnesium stearate is present in an amount of about 0.2-1% by weight. In some embodiments, a lubricant such as magnesium stearate is present in an amount of about 0.2-0.75% by weight. In some embodiments, a lubricant such as magnesium stearate is present in an amount of about 0.3% by weight. In some embodiments, a lubricant such as magnesium stearate is present in an amount of about 0.4% by weight. In some embodiments, a lubricant such as magnesium stearate is present in an amount of about 0.5% by weight. In some embodiments, a lubricant such as magnesium stearate is present in an amount of about 0.6% by weight. In some embodiments, a lubricant such as magnesium stearate is present in an amount of about 0.7% by weight. In some embodiments, a lubricant is present in an amount of from about 0.01 mg to 0.05 mg, 0.05 mg to 0.1 mg, 0.1 mg to 0.2 mg, 0.2 mg to 0.25 mg, 0.25 mg to 0.5 mg, 0.5 mg to 0.75 mg, 0.7 mg to 0.95 mg, 0.9 mg to 1.15 mg, 1.1 mg to 1.35 mg, 1.3 mg to 1.5 mg, 1.5 mg to 1.75 mg, 1.75 to 1.95 mg, 1.9 mg to 2.15 mg, 2.1 mg to 2.35 mg, 2.3 mg to 2.55 mg, 2.5 mg to 2.75 mg, 2.7 mg to 3.0 mg, 2.9 mg to 3.15 mg, 3.1 mg to 3.35 mg, 3.3 mg to 3.5 mg, 3.5 mg to 3.75 mg, 3.7 mg to 4.0 mg, 4.0 mg to 4.5 mg, 4.5 mg to 5.0 mg, 5.0 mg to 5.5 mg, 5.5 mg to 6.0 mg, 6.0 mg to 6.5 mg, 6.5 mg to 7.0 mg, 7.0 mg to 7.5 mg, 7.5 mg to 8.0 mg, 8.0 mg to 8.5 mg, 8.5 mg to 9.0 mg, 9.0 mg to 9.5 mg, or 9.5 mg to 10.0 mg. In some embodiments, a lubricant is present in an amount of about 0.01 mg, 0.05 mg, 0.1 mg, 0.2 mg, 0.25 mg, 0.5 mg, 0.7 mg, 0.9 mg, 1.1 mg, 1.3 mg, 1.5 mg, 1.7 mg, 1.9 mg, 2.mg, 2.3 mg, 2.5 mg, 2.75 mg, 3.0 mg, 3.1 mg, 3.3 mg, 3.5 mg, 3.7 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, or 10.0 mg.

Various useful glidants include, but are not limited to, tribasic calcium phosphate (TRI-TAB™), calcium silicate, cellulose, powdered (Sanacel™, Solka-Floe™), colloidal silicon dioxide (Aerosil™, Cab-O-Sil M-5P™, Wacker HDK™), magnesium silicate, magnesium trisilicate, starch (Melojel™, Meritena™, Paygel 55™, Perfectamyl D6PH™, Pure-Bind™, Pure-Cote™, Pure-Dent™, Pure-Gel™, Pure-Set™, Purity 21™, Purity 826™, Tablet White™) and talc (Luzenac Pharma™, Magsil Osmanthus™, Magsil Star™, Superiore™), or mixtures thereof. In some embodiments, a glidant is optionally used in an amount of about 0-15% by weight. In some embodiments, a glidant is present in an amount of from about 0.1 mg to 0.5 mg, 0.5 mg to 1 mg, 1 mg to 2 mg, 2 mg to 2.5 mg, 2.5 mg to 5 mg, 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23.5 mg, 23 mg to 25.5 mg, 25 mg to 27.5 mg, 27 mg to 30 mg, 29 mg to 31.5 mg, 31 mg to 33.5 mg, 33 mg to 35.5 mg, 35 mg to 37.5 mg, 37 mg to 40 mg, 40 mg to 45 mg, 45 mg to 50 mg, 50 mg to 55 mg, 55 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg. In some embodiments, a glidant is present in an amount of about 0.1 mg, 0.5 mg, 1 mg, 2 mg, 2.5 mg, 5 mg, 7 mg, 9 mg, 11 mg, 13 mg, 15 mg, 17 mg, 19 mg, 21 mg, 23 mg, 25 mg, 27.5 mg, 30 mg, 31.5 mg, 33.5 mg, 35.5 mg, 37.5 mg, 40 mg, 45 mg, 50 mg, 55 mg, 60 mg, 65 mg, 70 mg, 75 mg, 80 mg, 85 mg, 90 mg, 95 mg, or 100 mg.

Pharmaceutically acceptable surfactants include, but are limited to both non-ionic and ionic surfactants suitable for use in pharmaceutical dosage forms. Ionic surfactants may include one or more of anionic, cationic or zwitterionic surfactants. Various useful surfactants include, but are not limited to, sodium lauryl sulfate, monooleate, monolaurate, monopalmitate, monostearate or another ester of olyoxyethylene sorbitane, sodium dioctylsulfosuccinate (DOSS), lecithin, stearyic alcohol, cetostearylic alcohol, cholesterol, polyoxyethylene ricin oil, polyoxyethylene fatty acid glycerides, poloxamer, or any other commercially available co-processed surfactant like SEPITRAP® 80 or SEPITRAP® 4000 and mixtures thereof. In some embodiments, surfactant is optionally used in an amount of about 0-5% by weight. In some embodiments, a surfactant is present in an amount of from about 0.1 mg to 0.5 mg, 0.5 mg to 1 mg, 1 mg to 2 mg, 2 mg to 2.5 mg, 2.5 mg to 5 mg, 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23.5 mg, 23 mg to 25.5 mg, 25 mg to 27.5 mg, 27 mg to 30 mg, 29 mg to 31.5 mg, 31 mg to 33.5 mg, 33 mg to 35.5 mg, 35 mg to 37.5 mg, 37 mg to 40 mg, 40 mg to 45 mg, 45 mg to 50 mg, 50 mg to 55 mg, 55 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg. In some embodiments, a surfactant is present in an amount of about 0.1 mg, 0.5 mg, 1 mg, 2 mg, 2.5 mg, 5 mg, 7 mg, 9 mg, 11 mg, 13 mg, 15 mg, 17 mg, 19 mg, 21 mg, 23 mg, 25 mg, 27.5 mg, 30 mg, 31.5 mg, 33.5 mg, 35.5 mg, 37.5 mg, 40 mg, 45 mg, 50 mg, 55 mg, 60 mg, 65 mg, 70 mg, 75 mg, 80 mg, 85 mg, 90 mg, 95 mg, or 100 mg.

In some embodiments, the formulation comprises a combination of excipients selected from: stearic acid and lactose; stearic acid and lactose monohydrate; stearic acid and calcium carbonate; stearic acid and calcium phosphate; stearic acid and dibasic calcium phosphate; stearic acid and calcium sulfate; stearic acid and microcrystalline cellulose; stearic acid and cellulose powder; stearic acid and dextrose; stearic acid and dextrates; stearic acid and dextran; stearic acid and starches; stearic acid and pregelatinized starch; stearic acid and sucrose; stearic acid and xylitol; stearic acid and lactitol; stearic acid and mannitol; stearic acid and sorbitol; stearic acid and sodium chloride; stearic acid and polyethylene glycol; sodium stearyl fumarate and lactose; sodium stearyl fumarate and lactose monohydrate; sodium stearyl fumarate and calcium carbonate; sodium stearyl fumarate and calcium phosphate; sodium stearyl fumarate and dibasic calcium phosphate; sodium stearyl fumarate and calcium sulfate; sodium stearyl fumarate and microcrystalline cellulose; sodium stearyl fumarate and cellulose powder; sodium stearyl fumarate and dextrose; sodium stearyl fumarate and dextrates; sodium stearyl fumarate and dextran; sodium stearyl fumarate and starches; sodium stearyl fumarate and pregelatinized starch; sodium stearyl fumarate and sucrose; sodium stearyl fumarate and xylitol; sodium stearyl fumarate and lactitol; sodium stearyl fumarate and mannitol; sodium stearyl fumarate and sorbitol; sodium stearyl fumarate and sodium chloride; sodium stearyl fumarate and polyethylene glycol; glyceryl behenate and lactose; glyceryl behenate and lactose monohydrate; glyceryl behenate and calcium carbonate; glyceryl behenate and calcium phosphate; glyceryl behenate and dibasic calcium phosphate; glyceryl behenate and calcium sulfate; glyceryl behenate and microcrystalline cellulose; glyceryl behenate and cellulose powder; glyceryl behenate and dextrose; glyceryl behenate and dextrates; glyceryl behenate and dextran; glyceryl behenate and starches; glyceryl behenate and pregelatinized starch; glyceryl behenate and sucrose; glyceryl behenate and xylitol; glyceryl behenate and lactitol; glyceryl behenate and mannitol; glyceryl behenate and sorbitol; glyceryl behenate and sodium chloride; glyceryl behenate and polyethylene glycol; calcium stearate and lactose; calcium stearate and lactose monohydrate; calcium stearate and calcium carbonate; calcium stearate and calcium phosphate; calcium stearate and dibasic calcium phosphate; calcium stearate and calcium sulfate; calcium stearate and microcrystalline cellulose; calcium stearate and cellulose powder; calcium stearate and dextrose; calcium stearate and dextrates; calcium stearate and dextran; calcium stearate and starches; calcium stearate and pregelatinized starch; calcium stearate and sucrose; calcium stearate and xylitol; calcium stearate and lactitol; calcium stearate and mannitol; calcium stearate and sorbitol; calcium stearate and sodium chloride; calcium stearate and polyethylene glycol; glycerin monostearate and lactose; glycerin monostearate and lactose monohydrate; glycerin monostearate and calcium carbonate; glycerin monostearate and calcium phosphate; glycerin monostearate and dibasic calcium phosphate; glycerin monostearate and calcium sulfate; glycerin monostearate and microcrystalline cellulose; glycerin monostearate and cellulose powder; glycerin monostearate and dextrose; glycerin monostearate and dextrates; glycerin monostearate and dextran; glycerin monostearate and starches; glycerin monostearate and pregelatinized starch; glycerin monostearate and sucrose; glycerin monostearate and xylitol; glycerin monostearate and lactitol; glycerin monostearate and mannitol; glycerin monostearate and sorbitol; glycerin monostearate and sodium chloride; glycerin monostearate and polyethylene glycol; glyceryl palmitostearate and lactose; glyceryl palmitostearate and lactose monohydrate; glyceryl palmitostearate and calcium carbonate; glyceryl palmitostearate and calcium phosphate; glyceryl palmitostearate and dibasic calcium phosphate; glyceryl palmitostearate and calcium sulfate; glyceryl palmitostearate and microcrystalline cellulose; glyceryl palmitostearate and cellulose powder; glyceryl palmitostearate and dextrose; glyceryl palmitostearate and dextrates; glyceryl palmitostearate and dextran; glyceryl palmitostearate and starches; glyceryl palmitostearate and pregelatinized starch; glyceryl palmitostearate and sucrose; glyceryl palmitostearate and xylitol; glyceryl palmitostearate and lactitol; glyceryl palmitostearate and mannitol; glyceryl palmitostearate and sorbitol; glyceryl palmitostearate and sodium chloride; glyceryl palmitostearate and polyethylene glycol; magnesium lauryl sulfate and lactose; magnesium lauryl sulfate and lactose monohydrate; magnesium lauryl sulfate and calcium carbonate; magnesium lauryl sulfate and calcium phosphate; magnesium lauryl sulfate and dibasic calcium phosphate; magnesium lauryl sulfate and calcium sulfate; magnesium lauryl sulfate and microcrystalline cellulose; magnesium lauryl sulfate and cellulose powder; magnesium lauryl sulfate and dextrose; magnesium lauryl sulfate and dextrates; magnesium lauryl sulfate and dextran; magnesium lauryl sulfate and starches; magnesium lauryl sulfate and pregelatinized starch; magnesium lauryl sulfate and sucrose; magnesium lauryl sulfate and xylitol; magnesium lauryl sulfate and lactitol; magnesium lauryl sulfate and mannitol; magnesium lauryl sulfate and sorbitol; magnesium lauryl sulfate and sodium chloride; magnesium lauryl sulfate and polyethylene glycol; mineral oil and lactose; mineral oil and lactose monohydrate; mineral oil and calcium carbonate; mineral oil and calcium phosphate; mineral oil and dibasic calcium phosphate; mineral oil and calcium sulfate; mineral oil and microcrystalline cellulose; mineral oil and cellulose powder; mineral oil and dextrose; mineral oil and dextrates; mineral oil and dextran; mineral oil and starches; mineral oil and pregelatinized starch; mineral oil and sucrose; mineral oil and xylitol; mineral oil and lactitol; mineral oil and mannitol; mineral oil and sorbitol; mineral oil and sodium chloride; mineral oil and polyethylene glycol; palmitic acid and lactose; palmitic acid and lactose monohydrate; palmitic acid and calcium carbonate; palmitic acid and calcium phosphate; palmitic acid and dibasic calcium phosphate; palmitic acid and calcium sulfate; palmitic acid and microcrystalline cellulose; palmitic acid and cellulose powder; palmitic acid and dextrose; palmitic acid and dextrates; palmitic acid and dextran; palmitic acid and starches; palmitic acid and pregelatinized starch; palmitic acid and sucrose; palmitic acid and xylitol; palmitic acid and lactitol; palmitic acid and mannitol; palmitic acid and sorbitol; palmitic acid and sodium chloride; palmitic acid and polyethylene glycol; myristic acid and lactose; myristic acid and lactose monohydrate; myristic acid and calcium carbonate; myristic acid and calcium phosphate; myristic acid and dibasic calcium phosphate; myristic acid and calcium sulfate; myristic acid and microcrystalline cellulose; myristic acid and cellulose powder; myristic acid and dextrose; myristic acid and dextrates; myristic acid and dextran; myristic acid and starches; myristic acid and pregelatinized starch; myristic acid and sucrose; myristic acid and xylitol; myristic acid and lactitol; myristic acid and mannitol; myristic acid and sorbitol; myristic acid and sodium chloride; myristic acid and polyethylene glycol; poloxamer and lactose; poloxamer and lactose monohydrate; poloxamer and calcium carbonate; poloxamer and calcium phosphate; poloxamer and dibasic calcium phosphate; poloxamer and calcium sulfate; poloxamer and microcrystalline cellulose; poloxamer and cellulose powder; poloxamer and dextrose; poloxamer and dextrates; poloxamer and dextran; poloxamer and starches; poloxamer and pregelatinized starch; poloxamer and sucrose; poloxamer and xylitol; poloxamer and lactitol; poloxamer and mannitol; poloxamer and sorbitol; poloxamer and sodium chloride; poloxamer and polyethylene glycol; polyethylene glycol and lactose; polyethylene glycol and lactose monohydrate; polyethylene glycol and calcium carbonate; polyethylene glycol and calcium phosphate; polyethylene glycol and dibasic calcium phosphate; polyethylene glycol and calcium sulfate; polyethylene glycol and microcrystalline cellulose; polyethylene glycol and cellulose powder; polyethylene glycol and dextrose; polyethylene glycol and dextrates; polyethylene glycol and dextran; polyethylene glycol and starches; polyethylene glycol and pregelatinized starch; polyethylene glycol and sucrose; polyethylene glycol and xylitol; polyethylene glycol and lactitol; polyethylene glycol and mannitol; polyethylene glycol and sorbitol; polyethylene glycol and sodium chloride; polyethylene glycol and polyethylene glycol; sodium benzoate and lactose; sodium benzoate and lactose monohydrate; sodium benzoate and calcium carbonate; sodium benzoate and calcium phosphate; sodium benzoate and dibasic calcium phosphate; sodium benzoate and calcium sulfate; sodium benzoate and microcrystalline cellulose; sodium benzoate and cellulose powder; sodium benzoate and dextrose; sodium benzoate and dextrates; sodium benzoate and dextran; sodium benzoate and starches; sodium benzoate and pregelatinized starch; sodium benzoate and sucrose; sodium benzoate and xylitol; sodium benzoate and lactitol; sodium benzoate and mannitol; sodium benzoate and sorbitol; sodium benzoate and sodium chloride; sodium benzoate and polyethylene glycol; sodium chloride and lactose; sodium chloride and lactose monohydrate; sodium chloride and calcium carbonate; sodium chloride and calcium phosphate; sodium chloride and dibasic calcium phosphate; sodium chloride and calcium sulfate; sodium chloride and microcrystalline cellulose; sodium chloride and cellulose powder; sodium chloride and dextrose; sodium chloride and dextrates; sodium chloride and dextran; sodium chloride and starches; sodium chloride and pregelatinized starch; sodium chloride and sucrose; sodium chloride and xylitol; sodium chloride and lactitol; sodium chloride and mannitol; sodium chloride and sorbitol; sodium chloride and sodium chloride; sodium chloride and polyethylene glycol; sodium lauryl sulfate and lactose; sodium lauryl sulfate and lactose monohydrate; sodium lauryl sulfate and calcium carbonate; sodium lauryl sulfate and calcium phosphate; sodium lauryl sulfate and dibasic calcium phosphate; sodium lauryl sulfate and calcium sulfate; sodium lauryl sulfate and microcrystalline cellulose; sodium lauryl sulfate and cellulose powder; sodium lauryl sulfate and dextrose; sodium lauryl sulfate and dextrates; sodium lauryl sulfate and dextran; sodium lauryl sulfate and starches; sodium lauryl sulfate and pregelatinized starch; sodium lauryl sulfate and sucrose; sodium lauryl sulfate and xylitol; sodium lauryl sulfate and lactitol; sodium lauryl sulfate and mannitol; sodium lauryl sulfate and sorbitol; sodium lauryl sulfate and sodium chloride; sodium lauryl sulfate and polyethylene glycol; talc and lactose; talc and lactose monohydrate; talc and calcium carbonate; talc and calcium phosphate; talc and dibasic calcium phosphate; talc and calcium sulfate; talc and microcrystalline cellulose; talc and cellulose powder; talc and dextrose; talc and dextrates; talc and dextran; talc and starches; talc and pregelatinized starch; talc and sucrose; talc and xylitol; talc and lactitol; talc and mannitol; talc and sorbitol; talc and sodium chloride; talc and polyethylene glycol; zinc stearate and lactose; zinc stearate and lactose monohydrate; zinc stearate and calcium carbonate; zinc stearate and calcium phosphate; zinc stearate and dibasic calcium phosphate; zinc stearate and calcium sulfate; zinc stearate and microcrystalline cellulose; zinc stearate and cellulose powder; zinc stearate and dextrose; zinc stearate and dextrates; zinc stearate and dextran; zinc stearate and starches; zinc stearate and pregelatinized starch; zinc stearate and sucrose; zinc stearate and xylitol; zinc stearate and lactitol; zinc stearate and mannitol; zinc stearate and sorbitol; zinc stearate and sodium chloride; zinc stearate and polyethylene glycol; potassium benzoate and lactose; potassium benzoate and lactose monohydrate; potassium benzoate and calcium carbonate; potassium benzoate and calcium phosphate; potassium benzoate and dibasic calcium phosphate; potassium benzoate and calcium sulfate; potassium benzoate and microcrystalline cellulose; potassium benzoate and cellulose powder; potassium benzoate and dextrose; potassium benzoate and dextrates; potassium benzoate and dextran; potassium benzoate and starches; potassium benzoate and pregelatinized starch; potassium benzoate and sucrose; potassium benzoate and xylitol; potassium benzoate and lactitol; potassium benzoate and mannitol; potassium benzoate and sorbitol; potassium benzoate and sodium chloride; potassium benzoate and polyethylene glycol; magnesium stearate and lactose; magnesium stearate and lactose monohydrate; magnesium stearate and calcium carbonate; magnesium stearate and calcium phosphate; magnesium stearate and dibasic calcium phosphate; magnesium stearate and calcium sulfate; magnesium stearate and microcrystalline cellulose; magnesium stearate and cellulose powder; magnesium stearate and dextrose; magnesium stearate and dextrates; magnesium stearate and dextran; magnesium stearate and starches; magnesium stearate and pregelatinized starch; magnesium stearate and sucrose; magnesium stearate and xylitol; magnesium stearate and lactitol; magnesium stearate and mannitol; magnesium stearate and sorbitol; magnesium stearate and sodium chloride; and magnesium stearate and polyethylene glycol. Further excipients may also be present in the aforementioned formulation.

In some embodiments, a formulation comprises a combination of excipients selected from the aforementioned list. In some embodiments, a capsule comprises a formulation comprising a combination of excipients selected from the aforementioned list. In some embodiments, a gelatin capsule comprises a formulation comprising a combination of excipients selected from the aforementioned list. In some embodiments, a modified starch capsule comprises a formulation comprising a combination of excipients selected from the aforementioned list. In some embodiments, a carrageenan capsule comprises a formulation comprising a combination of excipients selected from the aforementioned list. In some embodiments, an HPMC capsule comprises a formulation comprising a combination of excipients selected from the aforementioned list.

### Dissolution

Drug dissolution represents a critical factor affecting the rate of systemic absorption. A variety of in vitro methods have been developed for assessing the dissolution properties of pharmaceutical formulations, and dissolution testing is sometimes used as a surrogate for the direct evaluation of drug bioavailability. See, e.g., Emmanuel et al., Pharmaceutics (2010), 2:351-363, and references cited therein. Dissolution testing measures the percentage of the API that has been released from the drug product (i.e., tablet or capsule) and dissolved in the dissolution medium under controlled testing conditions over a defined period of time. To maintain sink conditions, the saturation solubility of the drug in the dissolution media should be at least three times the drug concentration. For low solubility compounds, dissolution may sometimes be determined under non-sink conditions. Dissolution is affected by the properties of the API (e.g., particle size, crystal form, bulk density), the composition of the drug product (e.g., drug loading, excipients), the manufacturing process (e.g., compression forces) and the stability under storage conditions (e.g., temperature, humidity). The capsule dosage form prepared by the processes described herein can be subjected to in vitro dissolution evaluation according to Test 711 “Dissolution” in the United States Pharmacopoeia 37, United States Pharmacopoeial Convention, Inc., Rockville, Md., 2014 (“USP 711”) to determine the rate at which the active substance is released from the dosage form, and the content of the active substance can be determined in solution by high performance liquid chromatography. This test is provided to determine compliance with the dissolution requirements where stated in the individual monograph for dosage forms administered orally. In this general chapter, a dosage unit is defined as 1 tablet or 1 capsule or the amount specified. Of the types of apparatus described herein, use the one specified in the individual monograph. Where the label states that an article is enteric-coated, and where a dissolution or disintegration test that does not specifically state that it is to be applied to delayed-release articles is included in the individual monograph, the procedure and interpretation given for Delayed-Release Dosage Forms is applied unless otherwise specified in the individual monograph. For hard or soft gelatin capsules and gelatin-coated tablets that do not conform to the Dissolution specification, repeat the test as follows. Where water or a medium with a pH of less than 6.8 is specified as the Medium in the individual monograph, the same Medium specified may be used with the addition of purified pepsin that results in an activity of 750,000 Units or less per 1000 mL. For media with a pH of 6.8 or greater, pancreatin can be added to produce not more than 1750 USP Units of protease activity per 1000 mL.

### USP 711 Apparatus 1 (Basket Apparatus)

The assembly can comprise the following: a vessel, which may be covered, made of glass or other inert, transparent material; a motor; a metallic drive shaft; and a cylindrical basket. The vessel is partially immersed in a suitable water bath of any convenient size or heated by a suitable device such as a heating jacket. The water bath or heating device permits holding the temperature inside the vessel at 37±0.5 during the test and keeping the bath fluid in constant, smooth motion. No part of the assembly, including the environment in which the assembly is placed, contributes significant motion, agitation, or vibration beyond that due to the smoothly rotating stirring element. An apparatus that permits observation of the specimen and stirring element during the test is preferable. The vessel can be cylindrical, with a hemispherical bottom and with one of the following dimensions and capacities: for a nominal capacity of 1 L, the height can be 160 mm to 210 mm and its inside diameter can be 98 mm to 106 mm; for a nominal capacity of 2 L, the height can be 280 mm to 300 mm and its inside diameter can be 98 mm to 106 mm; and for a nominal capacity of 4 L, the height can be 280 mm to 300 mm and its inside diameter can be 145 mm to 155 mm. Its sides are flanged at the top. A fitted cover may be used to retard evaporation. The shaft can be positioned so that its axis is not more than 2 mm at any point from the vertical axis of the vessel and rotates smoothly and without significant wobble that could affect the results. A speed-regulating device can be used that allows the shaft rotation speed to be selected and maintained at the specified rate given in the individual monograph, within 4%.

Shaft and basket components of the stirring element can be fabricated of stainless steel, type 316, or other inert material. A basket having a gold coating of about 0.0001 inch (2.5 μm) thick may be used. A dosage unit can be placed in a dry basket at the beginning of each test. The distance between the inside bottom of the vessel and the bottom of the basket can be maintained at 25±2 mm during the test.

### USP 711 Apparatus 2 (Paddle Apparatus)

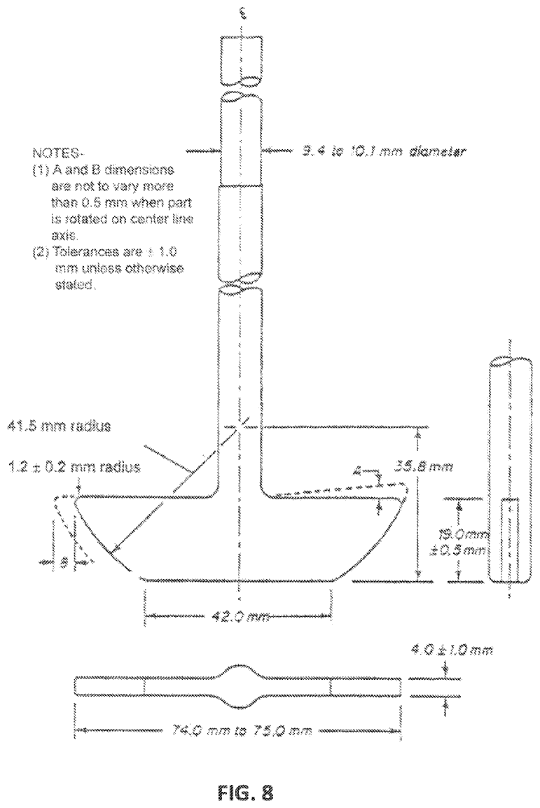

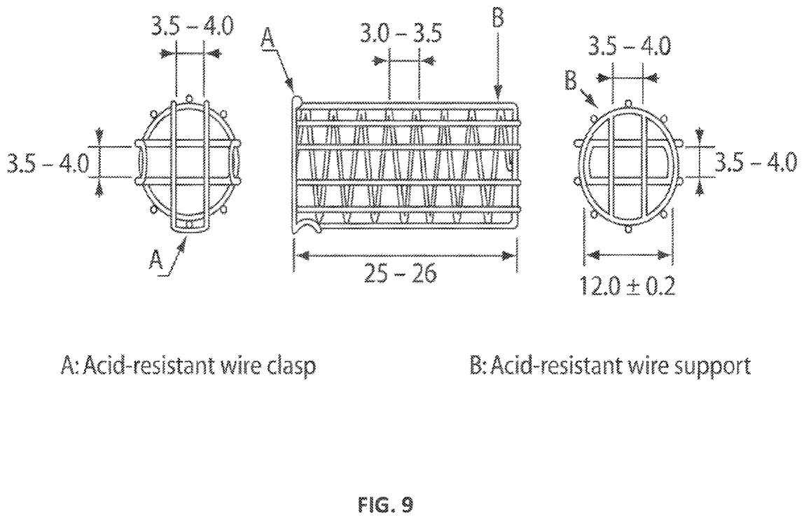

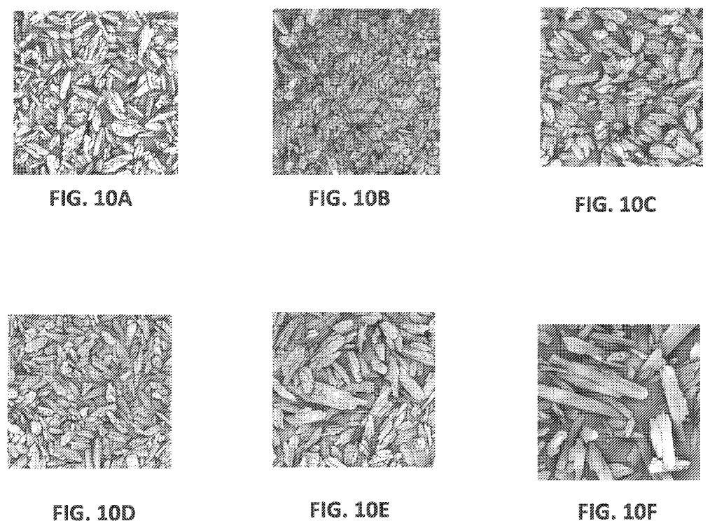

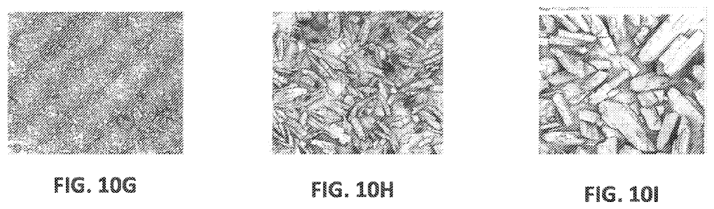

Use the assembly from Apparatus 1, except that a paddle formed from a blade and a shaft is used as the stirring element. The shaft is positioned so that its axis is not more than 2 mm from the vertical axis of the vessel at any point and rotates smoothly without significant wobble that could affect the results. The vertical center line of the blade passes through the axis of the shaft so that the bottom of the blade is flush with the bottom of the shaft. The paddle conforms to the specifications shown in FIG. 8. The distance of 25±2 mm between the bottom of the blade and the inside bottom of the vessel is maintained during the test. The metallic or suitably inert, rigid blade and shaft comprise a single entity. A suitable two-part detachable design may be used provided the assembly remains firmly engaged during the test. The paddle blade and shaft may be coated with a suitable coating so as to make them inert. The dosage unit is allowed to sink to the bottom of the vessel before rotation of the blade is started. A small, loose piece of nonreactive material, such as not more than a few turns of wire helix, may be attached to dosage units that would otherwise float. An alternative sinker device is shown in FIG. 9. Other validated sinker devices may be used.

When comparing the test and reference products, dissolution profiles can be compared using a similarity factor (f₂). The similarity factor is a logarithmic reciprocal square root transformation of the sum of squared error and is a measurement of the similarity in the percent (%) of dissolution between the two curves. Two dissolution profiles can be considered similar when the f₂ value is equal to or greater than 50.

f₂=50·log{[1+(1/n)Σₜ₌₁ⁿ(Rₜ-Tₜ)²]^{-0.5}·100}

In some aspects, dissolution rates are measured by a standard USP 2 rotating paddle apparatus as disclosed in USP 711, Apparatus 2. In some embodiments, the dosage form is added to a solution containing a buffer, e.g., phosphate, HCl, acetate, borate, carbonate, or citrate buffer. In some embodiments, the dosage form is added to a solution containing a buffer, e.g., phosphate, HCl, acetate, borate, carbonate, or citrate buffer, with a quantity of enzyme that results in a desired protease activity of dissolution medium. In some embodiments, at appropriate times following test initiation (e.g., insertion of the dosage form into the apparatus), filtered aliquots from the test medium are analyzed for niraparib by high performance liquid chromatography (HPLC). Dissolution results are reported as the percent of the total dose of niraparib tested dissolved versus time.

In some aspects, dissolution rates are measured by a standard USP 2 rotating paddle apparatus as disclosed in USP 711, Apparatus 2. In some embodiments, the dosage form is added to a solution containing a buffer, e.g., phosphate, HCl, acetate, borate, carbonate, or citrate buffer. In some embodiments, the dosage form is added to a solution with a pH of from 2-13, 3-12, 4-10, 5-9, 6-8, 4.1-5.5, or 5.8-8.8, e.g., a solution with a pH of 2, 3, 3.5 4, 4.1, 5, 5.8, 6, 7, 7.2, 7.5, 8, 8.3, 8.8, 9, 10, 11, 12, or 13. In some embodiments, the dosage form is added to a solution containing a buffer, e.g., phosphate, HCl, acetate, borate, carbonate, or citrate buffer, with a quantity of enzyme that results in the desired protease activity. In some embodiments, at appropriate times following test initiation (e.g., insertion of the dosage form into the apparatus), filtered aliquots from the test medium are analyzed for niraparib by high performance liquid chromatography (HPLC). Dissolution results are reported as the percent of the total dose of niraparib tested dissolved versus time. Dissolution rates of the compositions described herein can be consistent, for example, the dissolution of the compositions can be at least 90%, 95%, 98%, 99%, or 100% in 5, 10, 15, 30, 45, 60, or 90 minutes.

In some embodiments, the solid dosage form of any of the embodiments described herein, under dissolution evaluation, dissolves: not less than about 25%, 30%, 35%, 40%, 45%, 50%, 55%, 60%, 65%, 70%, 75%, 80%, 85%, 90%, 95%, 96%, 97%, 98%, 99%, or 100% of the niraparib in about 5 minutes. In some embodiments, the solid dosage form of any of the embodiments described herein, under the conditions of dissolution evaluation, dissolves: not less than about 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes.

In some embodiments, the solid dosage form of any of the embodiments described herein, under the conditions of dissolution evaluation, dissolves: not less than about 25%, 30%, 35%, 40%, 45%, 50%, 55%, 60%, 65%, 70%, 75%, 80%, 85%, 90%, 95%, 96%, 97%, 98%, 99%, or 100% of the niraparib in about 15 minutes.

In some embodiments, the solid dosage form of any of the embodiments described herein, under the conditions of dissolution evaluation, dissolves: not less than about 25%, 30%, 35%, 40%, 45%, 50%, 55%, 60%, 65%, 70%, 75%, 80%, 85%, 90%, 95%, 96%, 97%, 98%, 99%, or 100% of the niraparib in about 30 minutes.

In some embodiments, the solid dosage form of any of the embodiments described herein, under the conditions of dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 45 minutes.

In some embodiments, the solid dosage form of any of the embodiments described herein, under the conditions of dissolution evaluation, dissolves: not less than about 25%, 30%, 35%, 40%, 45%, 50%, 55%, 60%, 65%, 70%, 75%, 80%, 85%, 90%, 95%, 96%, 97%, 98%, 99%, or 100% of the niraparib in about 60 minutes.

In some embodiments, the solid dosage form of any of the embodiments described herein, under the conditions of dissolution evaluation, dissolves: not less than about 25%, 30%, 35%, 40%, 45%, 50%, 55%, 60%, 65%, 70%, 75%, 80%, 85%, 90%, 95%, 96%, 97%, 98%, 99%, or 100% of the niraparib in about 90 minutes.

In some embodiments, after being stored at 25° C./60% RH for about 3 months, the solid dosage form of any of the embodiments described herein, under dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 5 minutes.

In some embodiments, after being stored at about 25° C./60% RH for about 3 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes. In some embodiments, after being stored at about 25° C./60% RH for about 3 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 15 minutes

In some embodiments, after being stored at about 25° C./60% RH for about 3 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 30 minutes. In some embodiments, after being stored at about 25° C./60% RH for 3 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes.

In some embodiments, after being stored at about 25° C./60% RH for about 3 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 60 minutes. In some embodiments, after being stored at about 25° C./60% RH for 3 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 90 minutes.

In some embodiments, after being stored at about 25° C./60% RH for about 6 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 5 minutes. In some embodiments, after being stored at about 25° C./60% RH for 6 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes.

In some embodiments, after being stored at about 25° C./60% RH for about 6 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 15 minutes. In some embodiments, after being stored at about 25° C./60% RH for 6 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 30 minutes.

In some embodiments, after being stored at about 25° C./60% RH for about 6 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes.

In some embodiments, after being stored at about 25° C./60% RH for about 6 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 60 minutes. In some embodiments, after being stored at about 25° C./60% RH for 6 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 90 minutes.

In some embodiments, after being stored at 25° C./60% RH for about 9 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 5 minutes. In some embodiments, after being stored at about 25° C./60% RH for about 9 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes.

In some embodiments, after being stored at about 25° C./60% RH for about 9 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 15 minutes. In some embodiments, after being stored at about 25° C./60% RH for about 9 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 30 minutes.

In some embodiments, after being stored at about 25° C./60% RH for about 9 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes. In some embodiments, after being stored at about 25° C./60% RH for about 9 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 60 minutes.

In some embodiments, after being stored at 25° C./60% RH for about 9 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 90 minutes. In some embodiments, after being stored at about 25° C./60% RH for about 12 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 5 minutes.

In some embodiments, after being stored at about 25° C./60% RH for about 12 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes. In some embodiments, after being stored at about 25° C./60% RH for about 12 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 15 minutes.

In some embodiments, after being stored at about 25° C./60% RH for about 12 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes. In some embodiments, after being stored at about 25° C./60% RH for about 12 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes.

In some embodiments, after being stored at about 25° C./60% RH for about 12 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 0 minutes. In some embodiments, after being stored at about 25° C./60% RH for about 12 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 90 minutes.

In some embodiments, after being stored at about 25° C./60% RH for about 24 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 5 minutes. In some embodiments, after being stored at about 25° C./60% RH for about 24 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes.

In some embodiments, after being stored at about 25° C./60% RH for about 24 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 15 minutes. In some embodiments, after being stored at about 25° C./60% RH for about 24 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 30 minutes. In some embodiments, after being stored at about 25° C./60% RH for about 24 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes. In some embodiments, after being stored at about 5° C./60% RH for about 24 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 60 minutes. In some embodiments, after being stored at about 25° C./60% RH for about 24 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 90 minutes. In some embodiments, after being stored at about 25° C./60% RH for about 36 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 5 minutes. In some embodiments, after being stored at about 25° C./60% RH for about 36 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes. In some embodiments, after being stored at about 25° C./60% RH for about 36 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 15 minutes. In some embodiments, after being stored at about 25° C./60% RH for about 36 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 30 minutes.

In some embodiments, after being stored at about 25° C./60% RH for about 36 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 10 minutes. In some embodiments, after being stored at about 25° C./60% RH for about 36 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 60 minutes.

In some embodiments, after being stored at about 25° C./60% RH for about 36 months, the solid dosage form of any of the embodiments described herein, under the conditions of dissolution evaluation, dissolves: not less than about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in about 90 minutes.

### Stability

In some embodiments, the pharmaceutical composition disclosed herein is stable for at least about: 30 days, 60 days, 90 days, 6 months, 1 year, 18 months, 2 years, 3 years, 4 years, or 5 years, for example about 80%-100% such as about: 80%, 90%, 95%, or 100% of the active pharmaceutical agent in the pharmaceutical composition is stable, e.g., as measured by High Performance Liquid Chromatography (HPLC). In some embodiments, about 80%-100% (e.g., about: 90%-100% or 95-100%) of niraparib or a pharmaceutically acceptable salt thereof (e.g., niraparib tosylate monohydrate) in the pharmaceutical composition disclosed herein is stable for at least about: 30, 60, 90, 180, 360, 540, or 720 days, for example greater than 90 days, which can be measured by HPLC. In some embodiments, about: 80%, 85%, 90%, 95%, or 100% (e.g., about 95%) of the niraparib or a pharmaceutically acceptable salt thereof (e.g., niraparib tosylate monohydrate) is stable for 30 days or more, which can be measured by HPLC.

In some embodiments, the pharmaceutical formulations described herein are stable with respect to compound degradation (e.g., less than about 30% degradation, less than about 25% degradation, less than about 20% degradation, less than about 15% degradation, less than about 10% degradation, less than about 8% degradation, less than about 5% degradation, less than about 3% degradation, less than about 2% degradation, or less than about 1% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 11 months, at least about 12 months, at least about 24 months, or at least about 36 months under storage conditions (e.g., room temperature). In some embodiments, the formulations described herein are stable with respect to compound degradation over a period of at least about 1 week. In some embodiments, the formulations described herein are stable with respect to compound degradation over a period of at least about 1 month. In some embodiments, the formulations described herein are stable with respect to compound degradation over a period of at least about 3 months. In some embodiments, the formulations described herein are stable with respect to compound degradation over a period of at least about 6 months. In some embodiments, the formulations described herein are stable with respect to compound degradation over a period of at least about 9 months. In some embodiments, the formulations described herein are stable with respect to compound degradation over a period of at least about 12 months.

Methods for assessing the chemical storage stability of solid dosage forms under accelerated aging conditions have been described in the literature. See, e.g., S. T. Colgan, T. J. Watson, R. D. Whipple, R. Nosal, J. V. Beaman, D. De Antonis, “The Application of Science and Risk Based Concepts to Drug Substance Stability Strategies” J. Pharm. Innov. 7:205-2013 (2012); Waterman K C, Carella A J, Gumkowski M J, et al. Improved protocol and data analysis for accelerated shelf-life estimation of solid dosage forms. Pharm Res 2007; 24(4):780-90; and S. T. Colgan, R. J. Timpano, D. Diaz, M. Roberts, R. Weaver, K. Ryan, K. Fields, G. Scrivens, Opportunities for Lean Stability Strategies” J. Pharm. Innov. 9:259-271 (2014).

In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for about 1 month, 3 months, 6 months, 9 months, 12 months, 24 months, or 36 months at about 5° C. In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for about 1 month, 3 months, 6 months, 9 months, 12 months, 24 months, or 36 months at about 25° C. and about 60% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for about 1 month, 3 months, 6 months, 9 months, 12 months, 24 months, or 36 months at about 30° C. and about 65% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for about 1 month, 3 months, 6 months, 9 months, 12 months, 24 months, or 36 months at about 40° C. and about 75% relative humidity (RH).

In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of impurities (e.g., exemplary impurities described herein) after storage for about 1 month, 3 months, 6 months, 9 months, 12 months, 24 months, or 36 months at about 5° C. In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of known impurities after storage for about 1 month, 3 months, 6 months, 9 months, 12 months, 24 months, or 36 months at about 25° C. and about 60% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of known impurities after storage for about 1 month, 3 months, 6 months, 9 months, 12 months, 24 months, or 36 months at about 30° C. and about 65% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of known impurities after storage for about 1 month, 3 months, 6 months, 9 months, 12 months, 24 months, or 36 months at about 40° C. and about 75% relative humidity (RH).

In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for about 1 month, 3 months, 6 months, 9 months, 12 months, 24 months, or 36 months at about 5° C. In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for about 1 month, 3 months, 6 months, 9 months, 12 months, 24 months, or 36 months at about 25° C. and about 60% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for about 1 month, 3 months, 6 months, 9 months, 12 months, 24 months, or 36 months at about 30° C. and about 65% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for about 1 month, 3 months, 6 months, 9 months, 12 months, 24 months, or 36 months at about 40° C. and about 75% relative humidity (RH).

In some embodiments, the invention provides an oral dosage form comprising niraparib and a pharmaceutically acceptable carrier, wherein the dosage form exhibits less than about 3.0%, 2.5%, 2.0%, 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for about 1 month, 3 months, 6 months, 9 months, 12 months, 24 months, or 36 months at about 5° C. In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for about 1 month, 3 months, 6 months, 9 months, 12 months, 24 months, or 36 months at about 25° C. and about 60% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for about 1 month, 3 months, 6 months, 9 months, 12 months, 24 months, or 36 months at about 30° C. and about 65% relative humidity (RH). In some embodiments, the invention provides an oral dosage form comprising niraparib and a pharmaceutically acceptable carrier, wherein the dosage form exhibits less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for about 1 month, 3 months, 6 months, 9 months, 12 months, 24 months, or 36 months at about 40° C. and about 70% relative humidity (RH).

### Capsules

In some embodiments, the pharmaceutical composition is formulated into solid oral pharmaceutical dosage forms. Solid oral pharmaceutical dosage forms include, but are not limited to, tablets, capsules, powders, granules and sachets. For example, the solid oral pharmaceutical dosage form can be a capsule.

In some embodiments, a therapeutically effective amount of niraparib or a pharmaceutically acceptable salt thereof administered to a subject via a solid dosage form is in the range of about 1 mg to about 1000 mg. In some embodiments, a therapeutically effective amount of niraparib or a pharmaceutically acceptable salt thereof administered to a subject via a solid dosage form is in the range of from about 50 mg to about 300 mg. In some embodiments, a niraparib formulation is administered at an amount of about 50 mg to about 100 mg as a solid dosage form. In some embodiments, the niraparib formulation is administered at an amount of about 100 mg to about 300 mg as a solid dosage form. For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a therapeutically effective amount of niraparib tosylate monohydrate administered to a subject via a solid dosage form can be from about 1 mg to about 1000 mg, for example, from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a therapeutically effective amount of niraparib tosylate monohydrate administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a therapeutically effective amount of niraparib tosylate monohydrate administered to a subject via a solid dosage form can be about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some embodiments, a therapeutically effective amount of niraparib tosylate monohydrate administered to a subject via a solid dosage form is about 79.7 mg. In some embodiments, a therapeutically effective amount of niraparib tosylate monohydrate administered to a subject via a solid dosage form is about 159.4 mg. In some embodiments, a therapeutically effective amount of niraparib tosylate monohydrate administered to a subject via a solid dosage form is about 318.8 mg. In some embodiments, a therapeutically effective amount of niraparib tosylate monohydrate administered to a subject via a solid dosage form is about 478.2 mg. In some aspects, the solid oral dosage form can be administered one, two, or three times a day (b.i.d).

Contemplated compositions of the present invention provide a therapeutically effective amount of niraparib or a pharmaceutically acceptable salt thereof over an interval of about 30 minutes to about 8 hours after administration, enabling, for example, once-a-day, twice-a-day, three times a day, and etc. administration if desired.

The formulations described herein may be introduced into a suitable capsule by using an encapsulator, e.g., an encapsulator equipped with pellet dosing chamber. The capsule sizes may be 00, 00EL, 0, 0EL, 1, 1EL, 2, 2EL, 3, 4 or 5. In some embodiments, the particles in the capsule are in a size 0 or smaller, for example, a size 1 or smaller capsule.

In some aspects, the pharmaceutical composition disclosed herein is encapsulated into discrete units. In some embodiments, the discrete units are capsules or packets. In some embodiments, the pharmaceutical composition disclosed herein is enclosed in a capsule.

In some embodiments, the capsule is formed using materials which include, but are not limited to, natural or synthetic gelatin, pectin, casein, collagen, protein, modified starch, polyvinylpyrrolidone, acrylic polymers, cellulose derivatives, or combinations thereof. In some embodiments, the capsule is formed using preservatives, coloring and opacifying agents, flavorings and sweeteners, sugars, gastroresistant substances, or combinations thereof. In some embodiments, the capsule is coated. In some embodiments, the coating covering the capsule includes, but is not limited to, immediate release coatings, protective coatings, enteric or delayed release coatings, sustained release coatings, barrier coatings, seal coatings, or combinations thereof. In some embodiments, a capsule herein is hard or soft. In some embodiments, the capsule is seamless. In some embodiments, the capsule is broken such that the particulates are sprinkled on soft foods and swallowed without chewing. In some embodiments, the shape and size of the capsule also vary. Examples of capsule shapes include, but are not limited to, round, oval, tubular, oblong, twist off, or a non-standard shape. The size of the capsule may vary according to the volume of the particulates. In some embodiments, the size of the capsule is adjusted based on the volume of the particulates and powders. Hard or soft gelatin capsules may be manufactured in accordance with conventional methods as a single body unit comprising the standard capsule shape. A single-body soft gelatin capsule typically may be provided, for example, in sizes from 3 to 22 minims (1 minims being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size. In some embodiments, the pharmaceutical composition disclosed herein (e.g., capsule) is swallowed as a whole. In some embodiments, the pharmaceutical composition disclosed herein (e.g., capsule) does not completely disintegrate in mouth within about: 2, 3, 4, 5, 6, 7, 8, 9, 10, 11, 12, 13, 14, 15, 16, 17, 18, 19, or 20 minutes. In some embodiments, the pharmaceutical composition disclosed herein is not a film. In some embodiments, the pharmaceutical composition disclosed herein is not for buccal administration. In some embodiments, the pharmaceutical composition disclosed herein (e.g., capsule) dissolves in stomach or intestine.

In some embodiments, a capsule disclosed herein has a net weight ranging from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a capsule disclosed herein can have a net weight ranging from about 50 mg to 150 mg, from about 75 mg to about 125 mg, about 90 mg to about 110 mg, about 93 mg to about 107 mg, about 94 mg to about 106 mg, or about 95 mg to about 105 mg.

In some embodiments, a capsule disclosed herein has a net weight of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg. For example, a capsule disclosed herein can have a net weight of about 100 mg, about 98 mg, about 96 mg, about 94 mg, about 92 mg, about 90 mg, about 80 mg, about 70 mg, about 60 mg, or about 50 mg.

In some cases, a capsule has a volume ranging from about 0.1 to 0.9 ml, e.g., about 0.6 ml to about 0.8 ml, about 0.4 ml to about 0.6 ml, about 0.3 ml to about 0.5 ml, about 0.2 ml to about 0.4 ml, or about 0.1 ml to about 0.3 ml. In some cases, the capsule has a volume of about 0.9 ml, about 0.8 ml, about 0.7 ml, about 0.6 ml, about 0.5 ml, about 0.4 ml, about 0.35 ml, about 0.3 ml, about 0.25 ml, about 0.2 ml, about 0.15 ml, or about 0.1 ml. In some cases, a body of the capsule ranges from about 9 mm to about 20 mm long, e.g., about 17 mm to about 20 mm long, about 17 mm to about 19 mm long, about 16 mm to about 20 mm long, about 15 mm to about 19 mm long, about 14 mm to about 18 mm long, about 13 mm to about 17 mm long, about 12 mm to about 16 mm long, about 11 mm to about 15 mm long, about 10 mm to about 14 mm long, about 9 mm to about 13 mm long, about 9 mm to about 12 mm long, about 9 mm to about 11 mm long, or about 9 mm to about 10 mm long. In some cases, the body of the capsule is about 18 mm long, about 17 mm long, about 16 mm long, about 15 mm long, about 14 mm long, about 13 mm long, about 12 mm long, about 11 mm long, about 10 mm long, or about 9 mm long. In some cases, a cap of the capsule ranges from about 6 mm to about 12 mm long, e.g., about 10 mm to 12 mm long, about 9 mm to about 11 mm long, about 8 mm to about 10 mm long, about 7 mm to about 9 mm long, or about 6 mm to about 8 mm long. In some cases, the cap of the capsule is about 11 mm long, about 10 mm long, about 9 mm long, about 8 mm long, about 7 mm long, or about 6 mm long. In some cases, the body of the capsule has an external diameter ranging from about 4 mm to about 9 mm, e.g., about 6 mm to about 8 mm, about 7 mm to about 9 mm, about 7 mm to about 8 mm, about 5 mm to about 7 mm, or about 4 mm to about 6 mm. In some cases, the body of the capsule has an external diameter of about 9 mm, about 8 mm, about 7 mm, about 6 mm, about 5 mm, or about 4 mm. In some cases, a cap of the capsule has an external diameter ranging from about 4 mm to about 9 mm, e.g., about 7 mm to about 9 mm, about 6 mm to about 9 mm, about 7 mm to about 8 mm, about 5 mm to about 7 mm, or about 4 mm to about 6 mm. In some cases, the cap of the capsule has an external diameter of about 9 mm, about 8 mm, about 7 mm, about 6 mm, about 5 mm, or about 4 mm. In some cases, an overall closed length of the capsule ranges from about 10 mm to about 24 mm, e.g., about 20 mm to about 24 mm, or about 21 mm to about 23 mm, about 20 mm to about 22 mm, about 19 mm to about 21 mm, about 18 mm to about 20 mm, about 17 mm to about 19 mm, about 16 mm to about 18 mm, about 15 mm to about 17 mm, about 14 mm to about 16 mm, about 13 mm to about 15 mm, about 12 mm to about 14 mm, about 11 mm to about 13 mm, or about 10 mm to about 12 mm. In some cases, the overall closed length of the capsule is about 22 mm, about 24 mm, about 23 mm, about 21 mm, about 20 mm, about 19 mm, about 18 mm, about 17 mm, about 16 mm, about 15 mm, about 14 mm, about 13 mm, about 12 mm, about 11 mm, or about 10 mm.

In some cases, the capsule has a capacity of from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some cases, the capsule has a capacity of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

For example, the capsule can have a capacity of from about 50 mg to about 800 mg, e.g., about 400 mg to about 800 mg, about 350 mg to about 450 mg, about 300 mg to about 500 mg, about 300 mg to about 400 mg, about 250 mg to about 350 mg, about 200 mg to about 300 mg, about 200 mg to about 250 mg, about 150 mg to about 200 mg, about 100 mg to about 200 mg, about 100 mg to about 150 mg, about 50 mg to about 100 mg, about 600 g, about 500 mg, about 450 mg, about 425 mg, about 400 mg, about 375 mg, about 350 mg, about 325 mg, about 300 mg, about 275 mg, about 250 mg, about 225 mg, about 200 mg, about 175 mg, about 150 mg, about 125 mg, about 100 mg, or about 75 mg. In some cases, the capsule comprises a powder with a powder density of about 0.4 g/ml to about 1.6 g/ml, e.g., about 0.4 g/ml, g/ml 1.2 g/ml, g/ml 1 g/ml, or g/ml 0.8 g/ml. In some cases, the capsule is oblong.

The method can comprise administration of a niraparib composition in 1, 2, 3, or 4 capsules once, twice, or three times daily; for example 1 or 2 or 3 capsules.

In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., lactose monohydrate) is from about 1:10 to about 10:1, respectively, for example about 1:2, about 1:3, about 1:4, about 1:5, about 1:6, about 1:7, about 1:8, about 1:9, about 1:10, about 10:1, about 9:1, about 8:1, about 7:1, about 6:1, about 5:1, about 4:1, about 3:1, or about 2:1. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., magnesium stearate) is from about 10:1 to about 100:1, respectively, for example about 10:1, about 20:1, about 30:1, about 40:1, about 50:1, about 60:1, about 70:1, about 80:1, or about 90:1. In some embodiments, the weight ratio of a non-active pharmaceutical ingredient (e.g., lactose monohydrate or magnesium stearate) to an active pharmaceutical ingredient (e.g., niraparib or a pharmaceutically acceptable salt thereof such as niraparib tosylate monohydrate) to is from about 3:2 to about 11:1, from about 3:1 to about 7:1, from about 1:1 to about 5:1, from about 9:2 to about 11:2, from about 4:2 to about 6:2, about 5:1, or about 2.5:1. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., lactose monohydrate or magnesium stearate) is about 1:1.6. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., lactose monohydrate or magnesium stearate) is about 1:2. In some embodiments, the weight ratio of niraparib or a pharmaceutically acceptable salt thereof such as niraparib tosylate monohydrate to lactose monohydrate is about 38:61, for example, 38.32:61.18. In some embodiments, the weight ratio of niraparib or a pharmaceutically acceptable salt thereof such as niraparib tosylate monohydrate to magnesium stearate is about 77:1, for example, 76.64:1.

In some embodiments, the weight ratio of a first non-active pharmaceutical ingredient to a second non-active pharmaceutical ingredient is from about 5:1 to about 200:1, respectively, for example about 5:1, about 10:1, about 20:1, about 40:1, about 50:1, about 75:1, about 100:1, about 110:1, about 120:1, about 130:1, about 140:1, about 150:1, about 160:1, about 170:1, about 180:1, about 190:1, or about 200:1. In some embodiments, the weight ratio of lactose monohydrate to magnesium stearate is about 120:1 to about 125:1. In some embodiments, the weight ratio of lactose monohydrate to magnesium stearate is about 122.36:1.

### Indications Suitable for Treatment

Any subject having cancer, including breast cancer, ovarian cancer, cervical cancer, epithelial ovarian cancer, fallopian tube cancer, primary peritoneal cancer, endometrial cancer, prostate cancer, testicular cancer, pancreatic cancer, esophageal cancer, head and neck cancer, gastric cancer, bladder cancer, lung cancer (e.g., adenocarcinoma, NSCLC and SCLC), bone cancer (e.g., osteosarcoma), colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma (e.g., liposarcoma), bladder cancer, liver cancer (e.g., hepatocellular carcinoma), kidney cancer (e.g., renal cell carcinoma), myeloid disorders (e.g., AML, CML, myelodysplastic syndrome and promyelocytic leukemia), and lymphoid disorders (e.g., leukemia, multiple myeloma, mantle cell lymphoma, ALL, CLL, B-cell lymphoma, T-cell lymphoma, Hodgkin's lymphoma, non-Hodgkin's lymphoma, hairy cell lymphoma) may be treated with compounds and methods described herein.

In some embodiments, the methods of the invention treat subjects with ovarian cancer. In some embodiments, the methods of the invention treat subjects with epithelial ovarian cancer. In some embodiments, the methods of the invention treat subjects with fallopian tube cancer. In some embodiments, the methods of the invention treat subjects with primary peritoneal cancer.

In some embodiments, the methods of the invention treat subjects with recurrent ovarian cancer. In some embodiments, the methods of the invention treat subjects with recurrent epithelial ovarian cancer. In some embodiments, the methods of the invention treat subjects with recurrent fallopian tube cancer. In some embodiments, the methods of the invention treat subjects with recurrent primary peritoneal cancer.

In some embodiments, the methods of the invention treat subjects with recurrent ovarian cancer following a complete or partial response to a chemotherapy, such as a platinum-based chemotherapy. In some embodiments, the methods of the invention treat subjects with recurrent epithelial ovarian cancer following a complete or partial response to a chemotherapy, such as a platinum-based chemotherapy. In some embodiments, the methods of the invention treat subjects with recurrent fallopian tube cancer following a complete or partial response to a chemotherapy, such as a platinum-based chemotherapy. In some embodiments, the methods of the invention treat subjects with recurrent primary peritoneal cancer following a complete or partial response to a chemotherapy, such as a platinum-based chemotherapy.

In some embodiments, the methods of the invention treat subjects with recurrent ovarian cancer, recurrent epithelial ovarian cancer, recurrent fallopian tube cancer and/or recurrent primary peritoneal cancer following a complete or partial response to a platinum-based chemotherapy, wherein the subjects begin the treatment no later than 8 weeks after their most recent platinum-containing regimen. For example, subjects can begin treatment with niraparib about 7 weeks after their most recent platinum-containing regimen. For example, subjects can begin treatment with niraparib about 6 weeks after their most recent platinum-containing regimen. For example, subjects can begin treatment with niraparib about 6 weeks after their most recent platinum-containing regimen. For example, subjects can begin treatment with niraparib about 5 weeks after their most recent platinum-containing regimen. For example, subjects can begin treatment with niraparib about 4 weeks after their most recent platinum-containing regimen. For example, subjects can begin treatment with niraparib about 3 weeks after their most recent platinum-containing regimen. For example, subjects can begin treatment with niraparib about 2 weeks after their most recent platinum-containing regimen. For example, subjects can begin treatment with niraparib about 1 week after their most recent platinum-containing regimen.

In some embodiments, the methods of the invention treat subjects with prostate cancer

In some embodiments, the methods of the invention treat subjects with a pediatric cancer. Exemplary pediatric cancers include, but are not limited to adrenocortical carcinoma, astrocytoma, atypical teratoid rhabdoid tumor, brain tumors, chondroblastoma, choroid plexus tumor, craniopharyngioma, desmoid tumor, dysembryplastic neuroepithelial tumor (DNT), ependymoma, fibrosarcoma, germ cell tumor of the brain, glioblastoma multiforme, diffuse pontine glioma, low grade glioma, gliomatosis cerebri, hepatoblastoma, histiocytosis, kidney tumor, acute lymphoblastic leukemia (ALL), acute myeloid leukemia (AML), chronic myelogenous leukemia (CML), liposarcoma, liver cancer, Burkitt lymphoma, Hodgkin lymphoma, non-Hodgkin lymphoma, malignant fibrous histiocytoma, melanoma, myelodysplastic syndrome, nephroblastoma, neuroblastoma, neurofibrosarcoma, osteosarcoma, pilocytic astrocytoma, retinoblastoma, rhabdoid tumor of the kidney, rhabdomyosarcoma, Ewing sarcoma, soft tissue sarcoma, synovial sarcoma, spinal cord tumor and Wilm's tumor.

In some embodiments, the methods of the invention treat subjects with a cancer with a dosage of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg of niraparib or pharmaceutically acceptable salt thereof once-daily, twice-daily, or thrice-daily. In some embodiments, the methods of the invention treat subjects with a cancer with a dosage of about 150 mg to 175 mg, 170 mg to 195 mg, 190 mg to 215 mg, 210 mg to 235 mg, 230 mg to 255 mg, 250 mg to 275 mg, 270 to 295 mg, 290 mg to 315 mg, 310 mg to 335 mg, 330 mg to 355 mg, 350 mg to 375 mg, or 370 mg to 400 mg of niraparib or pharmaceutically acceptable salt thereof once-daily, twice-daily, or thrice-daily. In some embodiments, the methods of the invention treat subjects with a cancer with a dosage of 5 mg, 7.5 mg, 10 mg, 12.5 mg, 15 mg. 17.5 mg, 20 mg, 22.5 mg, 25 mg, 27.5 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of niraparib or pharmaceutically acceptable salt thereof once-daily, twice-daily, or thrice-daily.

In some embodiments, the methods of the invention treat subjects with a cancer with a dosage of from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg of niraparib or pharmaceutically acceptable salt thereof once-daily, twice-daily, or thrice-daily. In some embodiments, the methods of the invention treat subjects with a cancer with a dosage of from about 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23/5 mg, 23 mg to 25.5 mg, 25 mg to 27.5 mg, 27 mg to 30 mg, 30 mg to 35 mg, 35 mg to 40 mg, 40 mg to 45 mg, 45 mg to 50 mg, 50 mg to 55 mg, 55 mg to 60 mg, 60 to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg of niraparib or pharmaceutically acceptable salt thereof once-daily, twice-daily, or thrice-daily.

### Administration of the Compositions

The recommended dose of the niraparib capsule formulations described herein (e.g., ZEJULA™) as monotherapy is three 100 mg capsules taken orally once daily, equivalent to a total daily dose of 300 mg. Patients may be encouraged to take their dose of ZEJULA™ at approximately the same time each day. Bedtime administration may be a potential method for managing nausea.

As described herein, doses of 1 to 1000 mg of niraparib or a pharmaceutically acceptable salt thereof may be administered for treatment of subjects, and methods and compositions described herein may comprise once-daily, twice-daily, or thrice-daily administration of a dose of up to 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg once-daily, twice-daily, or thrice-daily. In some embodiments, the dose of niraparib or pharmaceutically acceptable salt thereof is from 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg, once-daily, twice-daily, or thrice-daily. In some embodiments, the methods of the invention treat subjects with a cancer with a dosage of 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg of niraparib or pharmaceutically acceptable salt thereof once-daily, twice-daily, or thrice-daily.

In some embodiments, a total daily dose of niraparib or a pharmaceutically acceptable salt thereof of 1 mg to 1000 mg, for example, or 50 to 300 mg, is administered. In some embodiments, the total daily dose of niraparib or a pharmaceutically acceptable salt thereof administered exceeds 100 mg per day. In some embodiments, the total daily dose of niraparib or a pharmaceutically acceptable salt thereof administered exceeds 200 mg per day. In some embodiments, the total daily dose of niraparib or a pharmaceutically acceptable salt thereof administered exceeds 300 mg per day. In some embodiments, the total daily dose of niraparib or a pharmaceutically acceptable salt thereof administered exceeds 400 mg per day. In some embodiments, the total daily dose of niraparib or a pharmaceutically acceptable salt thereof administered exceeds 500 mg per day.

In some embodiments, the total daily dose of niraparib or a pharmaceutically acceptable salt thereof administered does not exceed 500 mg per day. In some embodiments, the total daily dose of niraparib or a pharmaceutically acceptable salt thereof administered does not exceed 300 mg per day. In some embodiments, the total daily dose of niraparib or a pharmaceutically acceptable salt thereof administered does not exceed 100 mg per day. In some embodiments, the total daily dose of niraparib or a pharmaceutically acceptable salt thereof administered does not exceed 50 mg per day. In some embodiments, the total daily dose of niraparib or pharmaceutically acceptable salt thereof is from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. the total daily dose of niraparib or a pharmaceutically acceptable salt thereof is about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

A therapeutically effective dose of niraparib or a pharmaceutically acceptable salt thereof may be about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg per day. In some embodiments, the amount of niraparib or a pharmaceutically acceptable salt thereof administered daily is from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg per day.

In some embodiments, the amount of niraparib or a pharmaceutically acceptable salt thereof administered one time daily is about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some embodiments, the amount of naraparib or a pharmaceutically acceptable salt thereof administered one time daily is about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

In some embodiments, the amount of niraparib or a pharmaceutically acceptable salt thereof administered two times daily is about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some embodiments, the amount of niraparib or a pharmaceutically acceptable salt thereof administered two times daily is about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

In some embodiments, the amount of niraparib or a pharmaceutically acceptable salt thereof administered three times daily is about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some embodiments, the amount of niraparib or a pharmaceutically acceptable salt thereof administered three times daily is about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

In some embodiments, the niraparib or a pharmaceutically acceptable salt thereof is present at a dose from about 1 mg to about 1000 mg, including, but not limited to, about 1 mg, 5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100, 105 mg, 110 mg, 115 mg, 120 mg, 120.5 mg, 121 mg, 121.5 mg, 122 mg, 122.5 mg, 123 mg, 123.5 mg, 124 mg, 124.5 mg, 125 mg, 125.5 mg, 126 mg, 126.5 mg, 127 mg, 127.5 mg, 128 mg, 128.5 mg, 129 mg, 129.5 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

In some embodiments, the niraparib or a pharmaceutically acceptable salt thereof is present at a dose from about 1 mg to 5 mg, 5 mg to 10 mg, 10 mg to 20 mg, 20 mg to 25 mg, 25 mg to 100 mg, 35 mg to 140 mg, 70 mg to 140 mg, 80 mg to 135 mg, 10 mg to 25 mg, 25 mg to 50 mg, 50 mg to 100 mg, 100 mg to 150 mg, 150 mg to 200 mg, 10 mg to 35 mg, 35 mg to 70 mg, 70 mg to 105 mg, 105 mg to 140 mg, 140 mg to 175 mg, or 175 mg to 200 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg.

### Frequency of Administration

In some embodiments, a composition disclosed herein is administered to an individual in need thereof once. In some embodiments, a composition disclosed herein is administered to an individual in need thereof more than once. In some embodiments, a first administration of a composition disclosed herein is followed by a second administration of a composition disclosed herein. In some embodiments, a first administration of a composition disclosed herein is followed by a second and third administration of a composition disclosed herein. In some embodiments, a first administration of a composition disclosed herein is followed by a second, third, and fourth administration of a composition disclosed herein. In some embodiments, a first administration of a composition disclosed herein is followed by a second, third, fourth, and fifth administration of a composition disclosed herein. In some embodiments, a first administration of a composition disclosed herein is followed by a drug holiday.

The number of times a composition is administered to an individual in need thereof depends on the discretion of a medical professional, the disorder, the severity of the disorder, and the individual's response to the formulation. In some embodiments, a composition disclosed herein is administered once to an individual in need thereof with a mild acute condition. In some embodiments, a composition disclosed herein is administered more than once to an individual in need thereof with a moderate or severe acute condition. In the case wherein the patient's condition does not improve, upon the doctor's discretion the administration of niraparib may be administered chronically, that is, for an extended period of time, including throughout the duration of the patient's life in order to ameliorate or otherwise control or limit the symptoms of the patient's disease or condition.

In some embodiments, the composition is administered at predetermined time intervals over an extended period of time. In some embodiments, the niraparib composition is administered once every day. In some embodiments, the niraparib composition is administered every other day. In some embodiments, the niraparib composition is administered over about 1 week, 2 weeks, 1 month, 2 months, 3 months, 6 months, 1 year, 2 years, 3 years, 4 years, 5 years, 6 years, 7 years, 8 years, 9 years, 10 years, 11 years, or 12-15 years.

In some embodiments, the niraparib composition is administered in doses having a dose-to-dose niraparib concentration variation of less than about 50%, less than about 40%, less than about 30%, less than about 20%, less than about 10%, or less than about 5%.

In the case wherein the patient's status does improve, upon the doctor's discretion the administration of the niraparib may be given continuously; alternatively, the dose of drug being administered may be temporarily reduced or temporarily suspended for a certain length of time (i.e., a “drug holiday”). The length of the drug holiday can vary between about 2 days and 1 year, including by way of example only, about 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. A first or second dose reduction during a drug holiday may be from 10%-100%, including by way of example only about 10%, 15%, 20%, 25%, 30%, 35%, 40%, 45%, 50%, 55%, 60%, 65%, 70%, 75%, 80%, 85%, 90%, 95%, and 100%. For example, a first or second dose reduction during a drug holiday may be a dose reduced from about 5 mg to 1 mg, 10 mg to 5 mg, 20 mg to 10 mg, 25 mg to 10 mg, 50 mg to 25 mg, 75 mg to 50 mg, 75 mg to 25 mg, 100 mg to 50 mg, 150 mg to 75 mg, 100 mg to 25 mg, 200 mg to 100 mg, 200 to 50 mg, 250 mg to 100 mg, 300 mg to 50 mg, 300 mg to 100 mg, 300 mg to 200 mg, 400 mg to 50 mg, 400 mg to 100 mg, 400 mg to 200 mg, 500 mg to 50 mg, 500 mg to 100 mg, 500 mg to 250 mg, 1000 mg to 50 mg, 1000 mg to 100 mg, or 1000 mg to 500 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a first or second dose reduction during a drug holiday may be a dose reduced by about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

Once improvement of the patient's condition has occurred, a maintenance niraparib dose is administered if necessary. Subsequently, the dosage or the frequency of administration, or both, is optionally reduced, as a function of the symptoms, to a level at which the improved symptoms of the disease, disorder or condition is retained. In certain embodiments, patients require intermittent treatment on a long-term basis upon any recurrence of symptoms.

### Particle Size

In some embodiments, the pharmaceutical composition disclosed herein comprises pluralities of particulates. In some embodiments, the pharmaceutical composition comprises a plurality of first particulates and a plurality of second particulates. In some embodiments, the plurality of first particulates comprises niraparib. In some embodiments, the plurality of second particulates comprises lactose monohydrate. In some embodiments, the pharmaceutical composition disclosed herein comprises a plurality of third particulates. In some embodiments the plurality of third particulates comprises magnesium stearate.

The particle size of niraparib particles can be an important factor which can effect bioavailability, blend uniformity, segregation, and flow properties. In general, smaller particle sizes of a drug increases the drug absorption rate of permeable drugs with substantially poor water solubility by increasing the surface area and kinetic dissolution rate. The particle size of niraparib can also affect the suspension or blend properties of the pharmaceutical formulation. For example, smaller particles are less likely to settle and therefore form better suspensions. In some embodiments, the niraparib may optionally be screened niraparib. In some embodiments, the niraparib is not screened.

The pharmaceutical compositions disclosed herein comprise niraparib particles. In various embodiments, the niraparib formulations, in aqueous dispersions or as dry powders (which can be administered directly, as a powder for suspension, or used in a solid dosage form), can comprise niraparib with compatible excipients.

Particle size reduction techniques include, by way of example, grinding, milling (e.g., air-attrition milling (jet milling), ball milling), coacervation, complex coacervation, high pressure homogenization, spray drying and/or supercritical fluid crystallization. In some instances, particles are sized by mechanical impact (e.g., by hammer mills, ball mill and/or pin mills). In some instances, particles are sized via fluid energy (e.g., by spiral jet mills, loop jet mills, and/or fluidized bed jet mills).

In some embodiments, target and maximum particle size, including particle size distribution, is determined through analytical sieving in accordance with USP <786> or other appropriately validated methods. Exemplary filters used in particulate size generation include, without limitation, #16, #18, #20, #25, #30 #40, #60, #80, #100, #120, #140, #160, #180, #200, #220, and #240 size mesh screens. Diameter of granules can be also determined using Retsch AS 200 magnetic sieve shaker at an amplitude of 30 to 90 Hz with time interval between 5 to 30 minutes {Refer: USP 29 <786> Particle size distribution estimation by analytical sieving).

In some embodiments, the niraparib particles have a tap density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, the niraparib particles have a bulk density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, about 10%, 50%, or 90% of the particles of an excipient by weight have a particle size of less than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, about 10%, 50%, or 90% of the particles of an excipient by weight have a particle size of more than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, about 10% of the lactose monohydrate particles by weight have a particle size of less than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, about 50% of the lactose monohydrate particles by weight have a particle size of less than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm. In some embodiments, about 90% of the lactose monohydrate particles by weight have a particle size of less than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, about 10% of the lactose monohydrate particles by weight have a particle size of more than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, about 50% of the lactose monohydrate particles by weight have a particle size of more than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, about 90% of the lactose monohydrate particles by weight have a particle size of more than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, the lactose monohydrate particles have a tap density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, the lactose monohydrate particles have a bulk density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, about 10% of the magnesium stearate particles by weight have a particle size of less than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, about 50% of the magnesium stearate particles by weight have a particle size of less than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm. In some embodiments, about 90% of the magnesium stearate particles by weight have a particle size of less than about 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, about 10% of the magnesium stearate particles by weight have a particle size of more than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, about 50% of the magnesium stearate particles by weight have a particle size of more than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, about 90% of the magnesium stearate particles by weight have a particle size of more than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, about 10% of the lactose monohydrate particles by weight have a particle size of from 5 μm to 1000 μm, from 20 μm to 1000 μm, from 50 μm to 1000 μm, from 75 μm to 1000 μm, from 100 μm to 1000 μm, from 250 μm to 1000 μm, from 500 μm to 1000 μm, or from 750 μm to 1000 μm. In some embodiments, about 50% of the lactose monohydrate particles by weight have a particle size of from 5 μm to 1000 μm, from 20 μm to 1000 μm, from 50 μm to 1000 μm, from 75 μm to 1000 μm, from 100 μm to 1000 μm, from 250 μm to 1000 μm, from 500 μm to 1000 μm, or from 750 μm to 1000 μm. In some embodiments, about 90% of the lactose monohydrate particles by weight have a particle size of from 5 μm to 1000 μm, from 20 μm to 1000 μm, from 50 μm to 1000 μm, from 75 μm to 1000 μm, from 100 μm to 1000 μm, from 250 μm to 1000 μm, from 500 μm to 1000 μm, or from 750 μm to 1000 μm.

In some embodiments, about 10% of the lactose monohydrate particles by weight have a particle size of from 5 μm to 500 μm, from 20 μm to 500 μm, from 50 μm to 500 μm, from 75 μm to 500 μm, from 100 μm to 500 μm, or from 250 μm to 500 μm. In some embodiments, 5 about 0% of the lactose monohydrate particles by weight have a particle size of from 5 μm to 500 μm, from 20 μm to 500 μm, from 50 μm to 500 μm, from 75 μm to 500 μm, from 100 μm to 500 μm, or from 250 μm to 500 μm. In some embodiments, about 90% of the lactose monohydrate particles by weight have a particle size of from 5 μm to 500 μm, from 20 μm to 500 μm, from 50 μm to 500 μm, from 75 μm to 500 μm, from 100 μm to 500 μm, or from 250 μm to 500 μm.

In some embodiments, about 10% of the lactose monohydrate particles by weight have a particle size of from 5 μm to 250 μm, from 20 μm to 250 μm, from 50 μm to 250 μm, from 75 μm to 250 μm, or from 100 μm to 250 μm. In some embodiments, about 50% of the lactose monohydrate particles by weight have a particle size of from 5 μm to 250 μm, from 20 μm to 250 μm, from 50 μm to 250 μm, from 75 μm to 250 μm, or from 100 μm to 250 μm. In some embodiments, about 90% of the lactose monohydrate particles by weight have a particle size of from 5 μm to 250 μm, from 20 μm to 250 μm, from 50 μm to 250 μm, from 75 μm to 250 μm, or from 100 μm to 250 μm.

In some embodiments, about 30%, 40%, 50%, 60%, 70%, or 80% of the lactose monohydrate particles by weight have a particle size of from about 53 μm to 500 μm.

A method of making a formulation comprising niraparib can comprise obtaining niraparib; obtaining lactose monohydrate that has been screened with a screen; combining the niraparib with the screened lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. In some embodiments, obtaining niraparib comprises obtaining niraparib that has been screened. In some embodiments, combining the niraparib with the screened lactose monohydrate comprises combining unscreened niraparib with the screened lactose monohydrate.

### Powder Characteristics

As used herein, “permeability” is a measure of the powder's resistance to air flow. The permeability test utilizes the vented piston to constrain the powder column under a range of applied normal stresses; while air is passed through the powder column. The relative difference in air pressure between the bottom and the top of the powder column is a function of the powder's permeability. Tests can be carried out under a range of normal stresses and air flow rates. Usually, a lower pressure drop is indicative of higher permeability and often, better flow properties.

As used herein, the “flow rate index” (or FRI) is a measure of a powder's sensitivity to variable flow rate and is obtained as the ratio of the total energy required to induce powder flow at 10 mm/s and 100 mm/s blade tip speed. A larger deviation from 1 indicates greater sensitivity of a powder to variable flow rate.

FRI=Flow Energy@10 mm/s/Flow Energy@100 mm/*

As used herein, “specific energy” or SE is a measure of the powder flow in low stress environment and is derived from the shear forces acting on the blades as they rotate upward through the powder. The SE is recorded as the flow energy of the powder normalized by its weight in mJ/g during the upward spiral movement of the blades in a FT4 Powder Rheometer describe above. A lower SE is an indication of a less cohesive powder and better flow properties.

As used herein, “flow function” or FF is a parameter commonly used to rank powder's flowability and is determined using a shear test. The data produced in the shear test represents the relationship between shear stress and normal stress, which can be plotted to define the powder's Yield Locus. Fitting Mohr stress circles to the yield locus identifies the Major Principle Stress (MPS) and Unconfined Yield Strength (UYS). Flow function is the ratio of Major Principle Stress (MPS) to the Unconfined Yield Strength (UYS):

FF=MPS/UYS.

Flow characteristics can be evaluated by different tests such as angle of repose, Carr's index, Hausner ratio or flow rate through an orifice. Measures that may be taken to ensure that the compositions according to the invention have good flow and dispersion properties involve the preparation or processing of the powder particles. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib has a Hausner's ratio of less than about 1.3 or less than about 1.7 or wherein the niraparib has a Hausner's ratio of less than about 1.3 or less than about 1.8. In some embodiments, the niraparib has a Hausner's ratio of about 1.4 or less. In some embodiments, the niraparib has a Hausner's ratio of about 1.48 or less. In some embodiments, the niraparib has a Hausner's ratio of about 1.38 or less. In some embodiments, the niraparib has a Hausner's ratio of about 1.3-1.7. In some embodiments, the average is about 1.5. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib has a Hausner's ratio of less than about 1.3 or less than about 1.7. In some embodiments, the niraparib has a Hausner's ratio of about 1.48 or less. In some embodiments, the niraparib has a Hausner's ratio of about 1.38 or less. In some embodiments, the niraparib has a Hausner's ratio of about 1.3-1.7 or a range of about 1.4-1.8. In some embodiments, the average can be about 1.5. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation in the capsule has a Hausner's ratio of about 1.8 or less. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation in the capsule has a Hausner's ratio of about 1.63 or less or wherein the formulation on the capsule has a Hausner's ratio in the range of about 1.18-1.63. In some embodiments, the Hausner's ratio is about an average of 1.41. Provided herein is a capsule comprising a formulation comprising an effective amount of niraparib to inhibit polyadenosine diphosphate ribose polymerase (PARP) when administered to a human, lactose monohydrate, and magnesium stearate; wherein the formulation in the capsule has a Hausner's ratio of about 1.7 or less. In some embodiments, the formulation in the capsule has a Hausner's ratio of about 1.67 or less. In some embodiments, the formulation in the capsule has a Hausner's ratio of about 1.64 or less. In some embodiments, the formulation in the capsule has a Hausner's ratio of about 1.52 or less. In some embodiments, the formulation in the capsule has a Hausner's ratio of about 1.47 or less. In some embodiments, the formulation in the capsule has a Hausner's ratio of about 1.43 or less. In some embodiments, the formulation in the capsule has a Hausner's ratio of about 1.41 or less. In some embodiments, the formulation in the capsule has a Hausner's ratio of about 1.3 or less. In some embodiments, a capsule comprises a formulation comprising an effective amount of niraparib to inhibit polyadenosine diphosphate ribose polymerase (PARP) when administered to a human, lactose monohydrate, and magnesium stearate; wherein the has a Hausner's ratio of about 1.7 or less. In some embodiments, the formulation has a Hausner's ratio of about 1.67 or less. In some embodiments, the formulation has a Hausner's ratio of about 1.64 or less. In some embodiments, the formulation has a Hausner's ratio of about 1.52 or less. In some embodiments, the formulation has a Hausner's ratio of about 1.47 or less. In some embodiments, the formulation has a Hausner's ratio of about 1.43 or less. In some embodiments, the formulation has a Hausner's ratio of about 1.41 or less. In some embodiments, the formulation has a Hausner's ratio of about 1.3 or less.

In certain embodiments, powder characterization described herein can be determined using a FT4 Powder Rheometer (Freeman Technology), e.g., a FT4 Powder Rheometer with the 25 mm vessel assembly having 23.5 mm diameter blades, vented piston, a segmented rotational shear cell accessory and a 10 or 25 ml borosilicate vessel. The FT4 Powder Rheometer is capable of quantitatively measuring the flowability characteristics of particulate compositions, and these measurements can be utilized to predict the characteristics of the particulate composition when being pneumatically conveyed, e.g., in a dilute phase. The FT4 Powder Rheometer includes a container for holding a powder sample and a rotor having a plurality of blades that is configured to move in the axial direction (e.g., vertically) through the powder sample while rotating the blades relative to the container. See, for example, U.S. Pat. No. 6,065,330 by Freeman et al., which is incorporated herein by reference in its entirety. Powder testing can be generally divided into three categories: dynamic tests, permeability test and shear test.

For example, dynamic testing can use the 23.5 mm diameter blades and a 25 mL powder sample in the borosiliate test vessel. Powder is filled into the vessel and the blades are simultaneously rotated and moved axially into the powder sample as the axial and rotational forces are measure and used to calculate the dynamic flowability parameters, such as flow rate index (FRI) and Specific Energy (SE).

For example, using an FT4 Powder samples various manufactured blends can be subjected to the following tests as described in the FT4 user manual and/or associated Freeman Technology literature: The FT4 Aeration test determines Basic Flowability Energy, Specific Energy, Conditioned Bulk Density, Aerated Energy, Aeration Ratio and Normalised Aeration Sensitivity. The standard 25 mm Aeration program can be optimized to achieve improved reproducibility over the Freeman method. The FT4 Permeability test determines the Pressure Drop at compaction pressures from 0.6 kPa to 15 kPa. The standard 25 mm Permeability program can be optimized to achieve improved reproducibility over the Freeman method. The FT4 Shear test can be performed using the standard 25 mm Shear 3 kPa program which determines incipient shear stress up to a compaction pressure of 3 kPa. The FT4 Compressibility test can be performed using the standard 25 mm Compressibility 1-15 kPa which determines percentage compressibility up to a compaction pressure of 15 kPa. For example, powder can be filled into a vessel. The powder bed with a vested piston can be exposed to varying normal stress increased stepwise, e.g., from 1 kPa to 15 kPa. The pressure drop across the powder bed can be measured while air is flushed through the powder at a constant velocity, e.g., 2 mm/s.

Shear testing can be used measure powder shear properties which involves the stress limit required to initiate a powder flow. The shear testing uses a segmented rotational shear cell head and a 10 ml powder sample in the borosilicate test vessel. Powder is filled into the vessel. The shear cell head is simultaneously rotated and moved axially under the powder sample at pre-determined normal stresses as the shear stresses are measured to calculate several parameters, including the flow function (FF). Usually, powders of low cohesion have higher FF and that represents better flow properties. The permeability test can measure the ease of air transmission through a bulk powder which can be related to the powder's flowability. For example, a permeability testing can use a vented piston with an aeration base and 10 mL powder sample in the borosilicated test vessel.

BFE and SE are determined by the FT4 Powder Rheometer using the Stability and Variable Flow Rate method (“the SVFR method”). The SVFR method includes seven test cycles using a stability method and four test cycles using a variable flow rate method, where each test cycle includes a conditioning step before the measurement is taken. The conditioning step homogenizes the compositions by creating a uniform low stress packing of particles throughout the sample, which removes any stress history or excess entrained air prior to the measurement. The stability method includes maintaining the blade tip speed at about 100 millimeters per second (mm/s) during the test cycles, whereas the variable flow rate method involves four measurements using different blade tip speeds, namely about 100 mm/s, about 70 mm/s, about 40 mm/s and about 10 mm/s. The test measures the energy required to rotate the blade through the powder from the top of the vessel to the bottom and to rotate the blade through the powder from the bottom to the top of the vessel.

BFE is the total energy measured during the seventh cycle during the stability method measurements of the SVFR method described above (i.e., at a tip speed set at 100 mm/s) while the blade is rotating from the top of the vessel to the bottom. The BFE is a measurement of the energy required to establish a particular flow pattern in a (conditioned) powder, which is established by a downward counter-clockwise motion of the blade that puts the powder under a compressive stress. The BFE, when considered in conjunction with other powder characteristics, can be used to predict the pneumatic conveyance properties of the compositions described herein. For some particulate compositions, the lower the BFE, the more easily the compositions described herein can be made to flow in a regular and invariable manner, e.g., without significant variations in line pressure. However, for the compositions having a small volume of very fine particles, the composition may be relatively uncompressible due to a lack of entrained air that would otherwise surround the fine particles. That is, the compositions disclosed herein may begin in a relatively efficient packing state, and therefore blade movement in the rheometer is not accommodated by the air pockets that exist in more cohesive powders, i.e., powders containing higher levels of very fine particles. This may result in more contact stress, and therefore a higher BFE than powders that include many very fine particles.

The SE is the converse of the BFE, in the sense that the flow pattern is generated by an upward, clockwise motion of the blade in the powder rheometer, generating gentle lifting and low stress flow of the composition. Specifically, SE is the total energy measured during the seventh cycle during the stability method measurements of the SVFR method described above (i.e., at a tip speed set at −100 mm/s) while the blade is rotating from the bottom of the vessel to the top. As with the BFE, the reduced number of very fine particles in the compositions described herein may create an efficient particle packing state and the SE will be increased as compared to the same or similar powder that includes a larger volume of very fine particles.

Conditioned Bulk Density (“CBD”) may also be measured with the FT4 Powder Rheometer using the SVFR method. Bulk density may be measured at various packing conditions, and measuring the mass of a precise volume of conditioned powder provides the CBD. The CBD of a composition having a low percentage of very fine particles, e.g., that has been classified to remove very fine particles, may be higher than the CBD of the same powder that includes a higher percentage of very fine particles (e.g., that has not been classified to remove very fine particles). Thus, a higher CBD may indicate the presence of fewer very fine-sized particles (e.g., <5 μm) in the composition.

AE is a measure of how much energy is required for a powder to become aerated, which is directly related to the cohesive strength of the powder (i.e., the tendency for particles to “stick” together). AE may be determined in the FT4 Powder Rheometer using the aeration test, which provides a precise air velocity to the base of the vessel containing the powder and measures the change in energy required to rotate the blades through the powder sample as the air velocity changes. During the aeration test, the air velocity (e.g., in mm/s) is varied over a range of from about 0.2 millimeters per second (mm/s) to about 2.0 mm/s, e.g., in 0.2 mm/s increments. As a general rule, the less cohesive, and therefore more easily fluidized the composition, the lower the AE, and the more easily the powder composition can be pneumatically conveyed.

Another measure of cohesiveness is the AR, which is a unitless quantity expressing the ratio of AE at zero air velocity to the AE at a given air velocity. If the AR is 1, then there is very little change in AE as the air velocity increases, and the composition is said to be cohesive. Powders with ARs of 2 to 20 are said to have average sensitivity to aeration, and most powders fall within this range. At an AR above 20, powders are considered sensitive to aeration. As a general rule, the larger the AR and the lower the AE, the less cohesive and therefore more easily fluidized and pneumatically conveyed the powder.

The pressure drop, measured by the Permeability test, is a measure of the resistance to air flow between particles and through the powder bed. Pressure drop may be measured with the FT4 Powder Rheometer using a Permeability test which measures the pressure drop across the powder bed as a function of the applied normal stress (kinematic) in kPa. The less the pressure drop that is measured, the more likely the powder is to flow when pneumatically conveyed. Typically, a powder with low permeability will generate a pressure drop of over 50 mbar from at about 15 kPa and at an air velocity of 0.5 mm/s. In contrast, permeable powders will barely register a pressure drop at this air velocity. Powder permeability can be associated to its tendency towards bridging or segregation which are highly undesired occurrences during the manufacture of drug product. The permeability number measures relative ease for air to travel through a conditioned powder bed; low number indicates high permeability and therefore less chances for bridging/segregation

Compressibility is another characteristic that can affect flowability and may be measured by the FT4 Powder Rheometer using the compressibility test. Compressibility is a measure of how bulk density increases on compression. The less compressible a powder is, the more likely it is to flow when pneumatically conveyed because there are more paths for air. In other words, free flowing materials tend to be insensitive to compressibility. For example, a highly compressible composition with lower flowability would be characterized by a compressibility of about 40% at 15 kPa; and a more flowable sample would have a compressibility of less than 20% at 15 kPa.

### Morphology

The three dimensional morphology can render the milled or annealed or screened niraparib particles or blended compositions of the present invention more suitable for drug product manufacturing, e.g., coating, mixing, compression, extrusion, etc. than unmilled or unannealed or unscreened niraparib particles or blended compositions.

The niraparib particles or blended compositions of the present invention can be prepared by any suitable processes known in the art. In certain embodiments, the niraparib particles or blended compositions of the present invention are prepared by a process described herein. The niraparib particles can have a needle shape in some embodiments. The niraparib particles can have a rod shape in some embodiments. In some embodiments, the niraparib particles are shaped like fine rods and plates and are birefringent under cross-polarized light.

An “aspect ratio” is the ratio of width divided by length of a particle.

“Elongation” is defined as 1-aspect ratio. Shapes symmetrical in all axes, such as circles or squares, will tend to have an elongation close to 0, whereas needle-shaped particles will have values closer to 1. Elongation is more an indication of overall form than surface roughness.

“Convexity” is a measurement of the surface roughness of a particle and is calculated by dividing the perimeter of an imaginary elastic band around the particle by the true perimeter of the particle. A smooth shape, regardless of form, has a convexity of 1 while a very ‘spiky’ or irregular object has a convexity closer to 0.

“Circularity” or “high sensitivity circularity” is a measurement of the ratio of the actual perimeter of a particle to the perimeter of a circle of the same area. A perfect circle has a circularity of 1 while a very narrow rod has a High Sensitivity (HS) Circularity close to 0. The higher the HS Circularity value the closer it is to a circle. Intuitively, circularity is a measure of irregularity or the difference from a perfect circle.

### Milling

In some embodiments, a composition described herein comprises unmilled, milled, or a mixture of milled and unmilled niraparib particles. In some embodiments, the niraparib particles of a composition described herein are unmilled niraparib particles. In some embodiments, the niraparib particles of a composition described herein are milled niraparib particles. In some embodiments, the niraparib particles of a composition described herein are wet milled particles.

In some embodiments, niraparib particles can be milled with a milling apparatus. Various milling apparatus are known in the art including for example wet mills, ball mills, rotary mills, and fluid air milling systems.

An embodiment of the inventive method comprises wet-milling niraparib to provide a wet-milled niraparib composition. “Wet-milling” can also be referred to as “media milling” or “wet-bead milling.” In an embodiment of the invention, the method comprises wet-milling the niraparib in any suitable manner. Exemplary mills that may be suitable for wet-milling include, but are not limited to, ball (or bead) mill, rod mill, hammer mill, colloid mill, fluid-energy mill, high-speed mechanical screen mill, and centrifugal classifier mill. The size and amount of milling media (e.g., beads) may be varied, as appropriate, depending on, e.g., the desired size of the niraparib particles and the duration of the milling. In some embodiments, the milling media (e.g., beads) may be from about 0.5 mm to about 10 mm. The method may comprise wet-milling using any suitable amount of milling media. In some embodiments, the milling media may comprise from about 30% to about 70% of the volume of the mill chamber.

The inventive method may comprise wet-milling the mixture for any suitable duration. The duration of the wet-milling may be varied, as appropriate, depending on, e.g., the desired size of the niraparib particles, the size and/or amount of beads, and/or batch size. In some embodiments of the invention, the duration of the wet-milling may be from about one minute or less to about 20 minutes or more. In some embodiments, the duration of the wet-milling may be from about 2 minutes to about 15 minutes. In an embodiment of the invention, a change in any one or more of milling speed (impeller/tip speed), size or amount of the milling media, rate the mixture is fed into the mill, the viscosity or temperature of the mixture, amount of niraparib in the mixture, and size or hardness of niraparib particles may change the duration of milling required to achieve the desired particle size.

In some embodiments which include wet-milling a mixture of niraparib and aqueous liquid carrier, the method comprises drying the wet-milled, niraparib composition having the desired niraparib particle size. The drying may be carried out in any suitable manner, including but not limited to, spray-drying. An embodiment of the method further comprises processing the wet-milled niraparib composition into any suitable pharmaceutical composition.

In some embodiments, a method may comprise reaerating the wet-milled niraparib composition. Deaerating is optional and in some embodiments, the method may lack a reaerating step. Deaerating may be performed in any suitable manner such as, e.g., by vacuuming the mixture.

In some embodiments, reaerating the wet-milled niraparib composition provides a first-pass, wet-milled niraparib composition. A “pass,” as used herein, comprises wet-milling once and reaerating once as described herein. The inventive methods may comprise any suitable number of passes. The number of passes is not limited and in some embodiments, the inventive methods may comprise one, two, three, four, five, six, seven, eight, nine, ten, or more passes. In this regard, the inventive method may comprise repeating the wet-milling and/or reaerating described herein one or more times. The number of passes may be varied, as appropriate, depending on the desired size of the niraparib particles, the starting size of the niraparib particles, the amount of niraparib in the mixture, the amount of liquid carrier, the rate at which the mixture is added to the mill, and/or the temperature of the milling chamber. In some embodiments, the method comprises sizing a sample of the wet-milled, niraparib composition following each pass to determine if the niraparib particles have the desired size range. If the niraparib particles are too large, the method may comprise repeating wet-milling for one or more additional passes. If the niraparib particles have an acceptable size, the method may comprise processing the wet-milled niraparib composition to provide a pharmaceutical composition.

The wet-milling of the inventive method, regardless of the number of passes, may provide niraparib particles having any suitable cumulative size distribution.

An embodiment of the inventive method comprises processing the wet-milled niraparib composition to provide a pharmaceutical composition. The processing of the inventive method may be in any suitable manner to provide any suitable dosage form. In some embodiments, processing the wet-milled niraparib composition comprises encapsulating the wet-milled niraparib composition to provide a capsule. The pharmaceutical compositions prepared by the methods of the present invention can be encapsulated using large-scale production methods. Suitable methods of encapsulation include plate processes, rotary die-processes, microencapsulation processes, and machine encapsulation processes as disclosed in Remington's.

Another embodiment of the invention provides a method of preparing a pharmaceutical composition comprising wet-milling niraparib particles in a liquid carrier to provide a wet-milled niraparib composition and processing the wet-milled niraparib composition to provide a pharmaceutical composition. The method comprises wet-milling and processing as described herein with respect to other aspects of the invention.

A ball mill is a cylindrical device used in grinding or mixing materials. Ball mills typically rotate around a horizontal axis, partially filled with the material to be ground in addition to any grinding medium if used. Different materials are used as media, including ceramic balls such as high density alumina media, flint pebbles and stainless steel balls. An internal cascading effect reduces the particulate material to a finer powder. Industrial ball mills can operate continuously, fed at one end and discharged at the other end. Large to medium-sized ball mills are mechanically rotated on their axis, but small ones normally consist of a cylindrical capped container that sits on two drive shafts with belts used to transmit rotary motion.

Rotary mills, are also referred to as burr mills, disk mills, and attrition mills, typically include two metal plates having small projections (i.e. burrs). Alternatively, abrasive stones may be employed as the grinding plates. One plate may be stationary while the other rotates, or both may rotate in opposite directions.

A fluid air milling system utilizes turbulent free jets in combination with a high efficiency centrifugal classifier in a common housing. A typical fluid air milling system includes an inlet, chamber with rotor, screen, and an outlet. Feed can be introduced into the common housing through either a double flapper valve or injector. Flooding the pulverizing zone to a level above the grinding nozzles forms the mill load. Turbulent free jets can be used to accelerate the particles for impact and breakage. After impact the fluid and size reduced particles leave the bed and travel upwards to the centrifugal classifier where rotor speed will define which size will continue with the fluid through the rotor and which will be rejected back to the particle bed for further size reduction. The high degree of particle dispersion leaving the pulverizing zone aids in the efficient removal of fine particles by the classifier. Operating parameters of rotor speed, nozzle pressure, and bed level allow for optimizing productivity, product size, and distribution shape (slope). A low-pressure air purge can be used to seal the gap between the rotor and the outlet plenum eliminating particles bypassing the rotor and allowing for close top size control.

As the particle size of a powder decreases, the surface area typically increases. However, as the particle size of a powder decreases, the tendency to form agglomerations can also increase. This tendency to form agglomerations can offset any benefits obtained by increasing the surface area.

In some embodiments, milled particles have a higher packing density (i.e. relative to the same particles unmilled). For example, the packing density can increase by 0.2, 0.4, 0.6, 0.8, 1.0 or 1.2 g/cc. An increase in packing density of even 5 or 10% can be particularly beneficial for reducing the volume of powdered materials for shipping. In some embodiments, the packing density of milled particles or particle blends is increased by at least 20% relative to the same particles or particle blends that are unmilled.

### Annealing

In some embodiments, a method of making a composition described herein, such as a niraparib capsule formulation, comprises annealing the niraparib particles one or more times. For example, a method of making a niraparib capsule formulation can comprise heating and cooling the niraparib particles one, two, three, four, five, or more times. In some embodiments, the niraparib particles are annealed after milling, such as wet milling.

Annealing can comprise heating and cooling niraparib particles. For example, annealing can comprises heating niraparib particles to a temperature of about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C. for about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, or 14 hours, followed by cooling the niraparib particles.

For example, after heating the niraparib particles, the niraparib particles can be cooled to a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. over a period of time. For example, after heating the niraparib particles, the niraparib particles can be cooled to a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. over a period of about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 15 hours, 15 hours, 17 hours, 18 hours, 19 hours, 20 hours, 21 hours, 22 hours, 23 hours, or 24 hours or longer.

For example, annealing can comprises heating niraparib particles to a temperature of about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C. followed by cooling the niraparib particles to a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. over a period of about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 15 hours, 15 hours, 17 hours, 18 hours, 19 hours, 20 hours, 21 hours, 22 hours, 23 hours, or 24 hours or longer.

In some embodiments, particles of a composition described herein, such as niraparib particles, are annealed (e.g., heated and cooled) one or more times. For example, the niraparib particles of a composition described herein can be heated and cooled one, two, three, four, five, or more times.

In some embodiments, annealed particles exhibit a lower total energy of powder flow (i.e. relative to the same particles unannealed). In some embodiments, particles annealed two or more times, such as two or three or four or five or more times, exhibit a lower total energy of powder flow (i.e. relative to the same particles unannealed or annealed once). This equates to less energy expenditure for handing (e.g., conveying and mixing) powdered materials. Annealing two or more times can lower the total energy of powder flow by about 5%, 10%, 20%, 30%, 40%, 50%, 60%, or greater.

The free-flowing powder can exhibit any one or combination of improved properties as just described. In some embodiments, the niraparib particles of the present invention have a three dimensional morphology.

Measurement of particle size for niraparib formulations described herein can use, for example, wet dispersion laser diffraction method for particle size determination using a Malvern Mastersizer 3000 Particle Size Analyzer equipped with the Hydro MV sample dispersion unit. The particle size analyzer can determine particle size using low-angle laser light scattering and calculates results in % volume based on equivalent spheres. Volume distributions for the D₁₀, D₅₀, D₉₀, D_{4,3}, and D_{3,2} can be determined. The suspension is added to the tank until the obscuration is in range, targeting a 10% obscuration. Measurements are taken once the obscuration remains consistent.

The percentage of thicker particles can be determined using an instrument that measures the size and shape of particles, such as by the technique of static image analysis, for example, a Malvern Instrument Morphologi G3. The intensity of light can be quantified by a grey scale factor which depends on the amount of light reaching the detector. The grey scale image of a particle ranges from 0 (black) to 255 (white) and it is related to the thickness of the particle. The lower the intensity value the darker the image therefore the thicker the particle. In certain embodiments, the niraparib particles or blended compositions of the present invention have greater than about 30%, greater than about 40%, greater than about 45% or greater than about 50% of the particles with intensity less than about 80. In one embodiment, about 30-100%, 30-90%, 30-80%, 30%-70%, 30-60%, 40-60% or 40-50% of the niraparib particles or blended compositions of the present invention have intensity less than about 80.

In some embodiments, milled or annealed or screened niraparib particles in blended compositions of the present invention are slightly more elongated, less circular and more edgy or rough, as indicated by lower aspect ratio, lower HS circularity and lower convexity values, respectively, than unmilled or unannealed or unscreened niraparib particles in blended compositions.

In some embodiments, the niraparib particles in blended compositions of the present invention have a circularity value in the range of less than about 0.8, 0.7, 0.6, 0.5, 0.4, 0.3, 0.2, or 0.1. In another embodiment, about 40% of the niraparib particles in blended compositions by accumulated volume has a circularity value in the range of about 0.1 to 0.6. In some embodiments, the niraparib particles in blended compositions of the present invention has an aspect ratio in the range of 0.55 to 1.0. In some embodiments, the niraparib particles in blended compositions of the present invention has a convexity value in the range 0.95 to 1.0.

### Internal Friction Angle

In some embodiments, an angle of internal friction between niraparib particles or between particles of a blended composition described herein can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees.

In some embodiments, an angle of internal friction between niraparib particles can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees.

In some embodiments, an angle of internal friction between particles of a blend of niraparib particles and lactose monohydrate particles can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees. In some embodiments, an angle of internal friction between particles of a blend of niraparib particles and lactose monohydrate particles can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0.

In some embodiments, an angle of internal friction between particles of a blend of niraparib particles, lactose monohydrate particles and magnesium stearate particles, can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees. In some embodiments, an angle of internal friction between particles of a blend of niraparib particles, lactose monohydrate particles and magnesium stearate particles, can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib in the capsule has an internal friction angle of about 29 degrees or higher or about 33.1 degrees or higher. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib has an internal friction angle of about 29 degrees or higher or about 33.1 degrees or higher. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation in the capsule has an internal friction angle of less than about 34 degrees or of less than about 37 degrees. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation has an internal friction angle of less than about 34 degrees or of less than about 37 degrees.

### Flow Function (FF) Ratio

In some embodiments, the Flow Function (FF) Ratio of niraparib particles or of particles of a blended composition described herein can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0.

In some embodiments, the Flow Function (FF) Ratio of niraparib particles can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

In some embodiments, the Flow Function (FF) Ratio of particles of a blend of niraparib particles and lactose monohydrate particles can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0. In some embodiments, the Flow Function (FF) Ratio of particles of a blend of niraparib particles (e.g., milled niraparib particles) and lactose monohydrate particles can be at least about 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0.

In some embodiments, the Flow Function (FF) Ratio of particles of a blend of niraparib particles, lactose monohydrate particles and magnesium stearate particles, can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0. In some embodiments, the Flow Function (FF) Ratio of particles of a blend of niraparib particles, lactose monohydrate particles and magnesium stearate particles, can be at least about 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib has a flow function ratio value of more than about 3.5 or more than about 6.4. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib has a flow function ratio value of more than about 3.5 or more than about 6.4. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation has a flow function ratio value of more than about 6.5 or more than about 14.4. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation has a flow function ratio value of more than about 6.5 or more than about 14.4.

### Wall Friction

A Wall Friction test can be used to provide a measurement of the sliding resistance between a powder and the surface of process equipment, such as an encapsulator or blender or hopper. This can be important for understanding discharge behavior from hoppers, continuity of flow in transfer chutes and tablet ejection forces. It is also useful when investigating whether a powder will adhere to the wall of process equipment and various other surfaces, such as the inside of sachets, capsules and other packaging material. The measurement principle is very similar to the shear cell test, but rather than shearing powder against powder, in this test a coupon of material representing the process equipment wall is sheared against the powder in question. The FT4 Wall Friction accessory allows for a range of coupons to be investigated, and bespoke surfaces can be manufactured if required. Data is typically represented as a plot of shear stress against normal stress, allowing the determination of Wall Friction Angle (phi). The greater the wall friction angle, the higher the resistance between the powder and wall coupon.

Hoppers are used extensively throughout the processing environment and whilst they are often considered to be simple systems, they are responsible for causing a great deal of process interruption and product quality issues. If a powder possesses properties that are not optimized for the hopper geometry and equipment surface, then flow from the hopper may be variable or even none existent. Data from shear cell and wall friction tests can be used to calculate the critical hopper dimensions to ensure good flow.

A Wall Friction test can be used to measure the sliding resistance between the powder and the surface of the process equipment. This is particularly important for understanding discharge behavior from hoppers, continuity of flow in transfer chutes and tablet ejection forces. It is also useful when investigating whether a powder will adhere to the wall of process equipment and various other surfaces, such as the inside of sachets, capsules and other packaging material.

The measurement principle is very similar to the shear cell test, but rather than shearing powder against powder, in this test a coupon of material representing the process equipment wall is sheared against the powder in question. The FT4 Wall Friction accessory allows for a range of coupons to be investigated. Wall Friction is typically represented as a plot of shear stress against normal stress, allowing the determination of Wall Friction Angle (phi). The greater the wall friction angle, the higher the resistance between the powder and wall coupon.

In some embodiments, the wall friction angle of niraparib particles or of particles of a blended composition described herein can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees. In some embodiments, the wall friction angle of niraparib particles or of particles of a blended composition described herein can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees at an Ra of about 0.05 or at an Ra of about 1.2.

In some embodiments, the wall friction angle of niraparib particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees. In some embodiments, the wall friction angle of niraparib particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees at an Ra of about 0.05 or at an Ra of about 1.2.

In some embodiments, the wall friction angle of particles of a blend of niraparib particles and lactose monohydrate particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees. In some embodiments, the wall friction angle of particles of a blend of niraparib particles and lactose monohydrate particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees at an Ra of about 0.05 or at an Ra of about 1.2. In some embodiments, the wall friction angle of particles of a blend of niraparib particles (e.g., milled niraparib particles) and lactose monohydrate particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees. In some embodiments, the wall friction angle of particles of a blend of niraparib particles (e.g., milled niraparib particles) and lactose monohydrate particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees at an Ra of about 0.05 or at an Ra of about 1.2.

In some embodiments, the wall friction angle of particles of a blend of niraparib particles, lactose monohydrate particles and magnesium stearate particles, can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees. In some embodiments, the wall friction angle of particles of a blend of niraparib particles, lactose monohydrate particles and magnesium stearate particles, can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees at an Ra of about 0.05 or at an Ra of about 1.2. In some embodiments, the wall friction angle of particles of a blend of niraparib particles, lactose monohydrate particles and magnesium stearate particles, can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees. In some embodiments, the wall friction angle of particles of a blend of niraparib particles, lactose monohydrate particles and magnesium stearate particles, can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, 28.0, 28.1, 28.2, 28.3, 28.4, 28.5, 28.6, 28.7, 28.8, 28.9, 29.0, 29.1, 29.2, 29.3, 29.4, 29.5, 29.6, 29.7, 29.8, 29.9 or 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9 or 36.0 degrees at an Ra of about 0.05 or at an Ra of about 1.2. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib has a wall friction angle of less than about 29 at an Ra of about 0.05 or of less than about 35 at an Ra of about 0.05. In some embodiments, a capsule comprises a formulation comprising an effective amount of niraparib to inhibit polyadenosine diphosphate ribose polymerase (PARP) when administered to a human, lactose monohydrate, and magnesium stearate; wherein the niraparib has a wall friction angle of less than about 29 at an Ra of about 0.05 or of less than about 35 at an Ra of about 0.05. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 15 degrees at an Ra of about 0.05 or of less than about 25 degrees at an Ra of about 0.05. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 15 degrees at an Ra of about 0.05 of less than about 25 degrees at an Ra of about 0.05. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 26 degrees at an Ra of about 1.2 or of less than about 30 degrees at an Ra of about 1.2. In some embodiments, a capsule comprises a formulation comprising an effective amount of niraparib to inhibit polyadenosine diphosphate ribose polymerase (PARP) when administered to a human, lactose monohydrate, and magnesium stearate; wherein the formulation has a wall friction angle of less than about 26 degrees at an Ra of about 1.2 or of less than about 30 degrees at an Ra of about 1.2.

### Compressibility

In some embodiments, the compressibility percentage measured at 15 kPa of particles of a composition, such as an unmilled or milled composition described herein, can be at most or at least about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%.

In some embodiments, the compressibility percentage measured at 15 kPa of milled or unmilled niraparib particles of a composition described herein can be at most or at least about 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%.

In some embodiments, the compressibility percentage measured at 15 kPa of unmilled or milled niraparib particles of a composition described herein that have been annealed once time can be at least about 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%. In some embodiments, the compressibility percentage measured at 15 kPa of unmilled or milled niraparib particles of a composition described herein that have been annealed once time can be at most about 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9%, 50.0%, or 60%.

In some embodiments, the compressibility percentage measured at 15 kPa of unmilled or milled niraparib particles of a composition described herein that have been annealed two or more times can be at least about 3.0%, 3.1%, 3.2%, 3.3%, 3.4%, 3.5%, 3.6%, 3.7%, 3.8%, 3.9%, 4.0%, 4.1%, 4.2%, 4.3% 4.4% 4.5% 4.6%, 4.7% 4.8%, 4.9% 5.0%, 5.1%, 5.2%, 5.3% 5.4% 5.5% 5.6%, 5.7%, 5.8%, 5.9% 6.0%, 6.1%, 6.2%, 6.3%, 6.4%, 6.5%, 6.6%, 6.7%, 6.8%, 6.9%, 7.0, 7.1%, 7.2%, 7.3% 7.4% 7.5% 7.6%, 7.7% 7.8%, 7.9% 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.50%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.10%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9% or 30.0%. In some embodiments, the compressibility percentage measured at 15 kPa of unmilled or milled niraparib particles of a composition described herein that have been annealed two or more times can be at most about 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9% or 30.0%.

In some embodiments, the compressibility percentage measured at 15 kPa of niraparib particles can be at most or at least about 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%.

In some embodiments, the compressibility percentage measured at 15 kPa of particles of a blend of niraparib particles and lactose monohydrate particles can be at most or at least about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.50%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9% or 20.0%. In some embodiments, the compressibility percentage measured at 15 kPa of a blend of niraparib particles (e.g., milled niraparib particles) and lactose monohydrate particles can be at most about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9% or 13.0%. In some embodiments, the compressibility percentage measured at 15 kPa of a blend of niraparib particles (e.g., milled niraparib particles) and lactose monohydrate particles can be at least about 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, or 17.0%.

In some embodiments, the compressibility percentage measured at 15 kPa of a blend of niraparib particles, lactose monohydrate particles and magnesium stearate particles can be at most or at least about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.10%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9% or 20.0%. In some embodiments, the compressibility percentage measured at 15 kPa of particles of a blend of niraparib particles, lactose monohydrate particles and magnesium stearate particles, can be at most about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 40.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9% or 13.0%.

In some embodiments, the compressibility percentage measured at 15 kPa of particles of a blend of niraparib particles, lactose monohydrate particles and magnesium stearate particles, can be at least about 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, or 17.0%.

### Methods of Making Niraparib Formulations

Provided herein are methods of manufacturing niraparib capsule compositions for treating cancers. Also described herein are niraparib capsule formulations containing niraparib tosylate monohydrate, lactose monohydrate and magnesium stearate formed by disclosed methods, and the therapeutic use of such formulation orally. The disclosed formulation can be a dry powder blend in a capsule containing niraparib as an active pharmaceutical ingredient (API), an excipient such as lactose monohydrate, and lubricant such as magnesium stearate. The niraparib capsule composition can contain 19.2˜38.3% w/w niraparib, 61.2˜80.3% w/w lactose, and at least 0.5% w/w magnesium stearate.

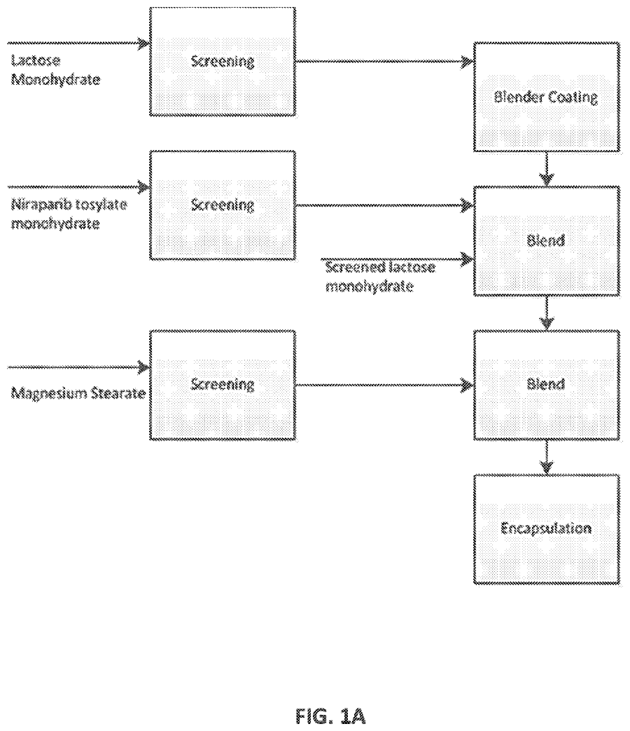

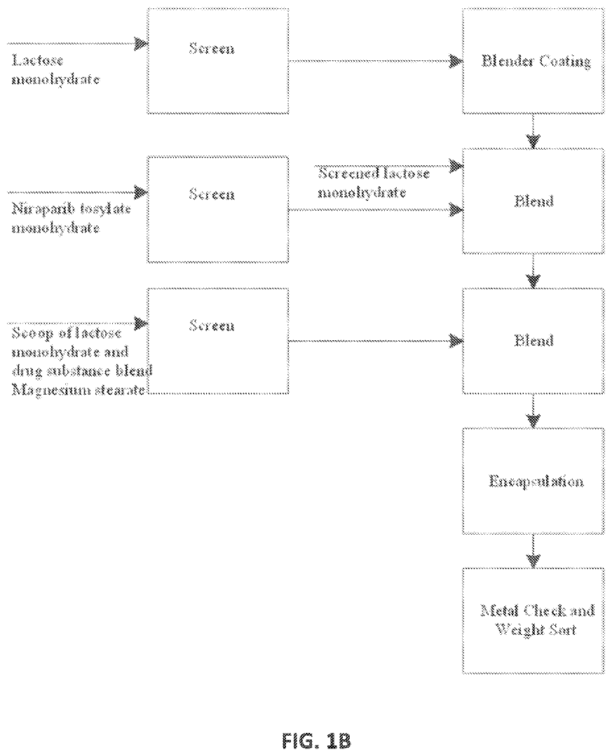

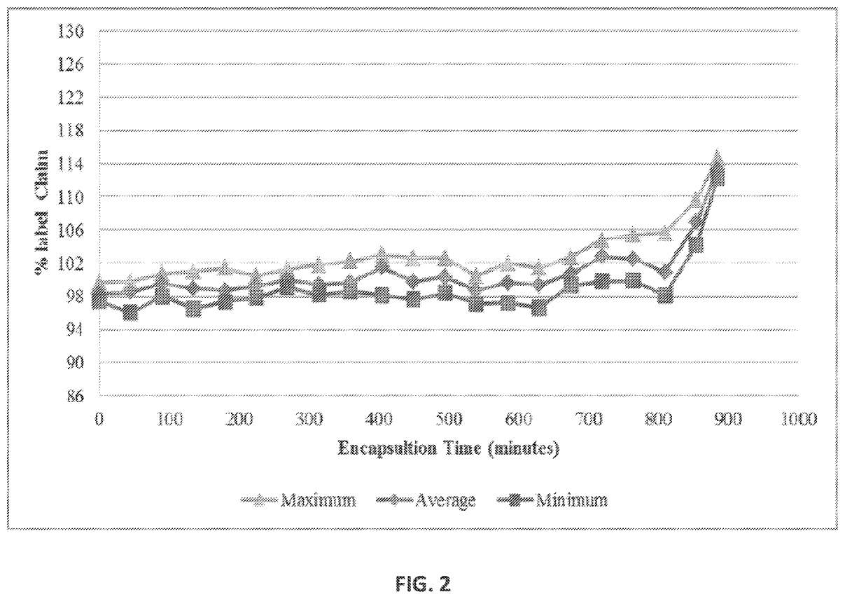

The manufacturing process can comprise blending screened lactose with niraparib followed by mixing and blending with screened magnesium stearate and further followed by encapsulation, wherein lactose is screened through a mesh screen, for example, having a mesh size of at most 600 microns, and magnesium stearate is screened through a mesh screen, for example, having a size of greater than 250 microns. The manufacturing process can comprise blending screened lactose with screened niraparib followed by mixing and blending with screened magnesium stearate and further followed by encapsulation, wherein lactose is screened through a mesh screen, for example, having a mesh size of at most 600 microns, and niraparib is screened through a mesh screen, for example, having a size of greater than 425 microns, and magnesium stearate is screened through a mesh screen, for example, having a size of greater than 250 microns. In some embodiments, the manufacturing process comprises obtaining screened lactose that has been screened through a mesh screen, for example, with a size of about 600 microns, and obtaining screened niraparib that has been screened through a mesh screen, for example, with a size of about 1180 microns, and obtaining screened magnesium stearate that has been screened through a mesh screen, for example, with a size of about 600 microns. An exemplary diagram showing the manufacturing process is shown in FIG. 1.

Different screening methods can be used for screening niraparib, for example, a conical mill, a vibratory sifter, or an oscillating screen where manufacturing process utilizes screened niraparib.

Various blenders can be used for blending the mixed compositions, for example, V-blender and double cone blender. Different blending conditions may be used for blenders having different sizes, including variations in size, speed, and time of blending.

In some embodiments, hold times between blending and encapsulation are about 1, 2, 3 or 4 days. In some embodiments, hold times between blending and encapsulation are less than 1, 2, 3 or 4 days.

A variety of encapsulators are used including manual, semi-automatic and full automatic encapsulators. In some embodiments, a manual encapsulation machine is used. And in some other embodiments, an automated encapsulator is used. In some embodiments, a Profill (Torpac, Fairfield, N.J.) manual encapsulation machine is used. And in some other embodiments, an automated Bosch GKF 330 powder filling encapsulator is used. The speed of the encapsulator can be adjusted to aid non-ideal powder flow. The encapsulator relies upon centrifugal force to move the powder from the hopper across the dosing bowl, where the powder then fills the holes in the dosing disc. Increasing the speed of the encapsulator increases the rotational velocity of the bowl and the associated centrifugal force. The increased force has the potential to improve the powder flow and reduce segregation.

In some embodiments, the speed of the encapsulator is greater than about 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 75,000, 100,000, 150,000 or 200,000 capsules/hour. In some embodiments, the speed of the encapsulator ranges from about 12,000 to 18,000 capsules/hour.

The height of the dosing disc can be set at a height lower than 17.5 mm to prevent overfill. During manufacturing, sticking on the tamping pins and the dosing disc was noted in certain batches. To mitigate the sticking potential, a coating can added to the tamping pins and dosing disc and screening of the drug substance can performed. The tamping pin and dosing disc can be coated with nickel and chrome coating which helps eliminate build-up and possible stickiness during encapsulation. To eliminate or reduce non-ideal powder flow and sticking during encapsulation that may have been the result of static charge, screening can be introduced to de-lump the drug substance. Due to the reduced mechanical agitation, the screening may reduce the potential for triboelectrification of the drug substance.

In some embodiments, the pharmaceutical composition of the present invention is prepared by blending the niraparib with excipients. The blending of above components can preferably be carried out in a mixer, for example in a tumble blender. Bulk density and tapped density can be determined according to USP 24, Test 616 “Bulk Density and Tapped Density”.

In some embodiments, the solid dosage forms of the present invention may be in the form of a powder (including a sterile packaged powder, a dispensable powder, or an effervescent powder), or a capsule (including both soft or hard capsules, e.g., capsules made from animal-derived gelatin or plant-derived HPMC, or “sprinkle capsules”). In some embodiments, the pharmaceutical formulation is in the form of a powder. Additionally, pharmaceutical formulations of the present invention may be administered as a single capsule or in multiple capsule dosage form. In some embodiments, the pharmaceutical formulation is administered in one, or two, or three, or four, capsules.

In some embodiments, solid dosage forms, e.g., capsules, are prepared by mixing niraparib particles with one or more pharmaceutical excipients to form a bulk blend composition. When referring to these bulk blend compositions as homogeneous, it is meant that the niraparib particles are dispersed evenly throughout the composition so that the composition may be readily subdivided into equally effective unit dosage forms, such as capsules. The individual unit dosages may also comprise film coatings, which disintegrate upon oral ingestion or upon contact with diluents.

Non-limiting pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The invention should not be considered limited to these particular conditions for combining the components and it will be understood, based on this disclosure that the advantageous properties can be achieved through other conditions provided the components retain their basic properties and substantial homogeneity of the blended formulation components of the formulation is otherwise achieved without any significant segregation.

In one embodiment for preparing the blend, the components are weighed and placed into a blending container. Blending is performed for a period of time to produce a homogenous blend using suitable mixing equipment. Optionally, the blend is passed through a mesh screen to delump the blend. The screened blend may be returned to the blending container and blended for an additional period of time. Lubricant may then be added and the blend mixed for an additional period of time.

In the pharmaceutical industry, milling is often used to reduce the particle size of solid materials. Many types of mills are available including cone mills, pin mills, hammer mills and jet mills. One of the most commonly used types of mill is the hammer mill. The hammer mill utilizes a high-speed rotor to which a number of fixed or swinging hammers are attached. The hammers can be attached such that either the knife face or the hammer face contacts the material. As material is fed into the mill, it impacts on the rotating hammers and breaks up into smaller particles. A screen is located below the hammers, which allows the smaller particles to pass through the openings in the screen. Larger particles are retained in the mill and continue to be broken up by the hammers until the particles are fine enough to flow through the screen. The material may optionally be screened. In screening, material is placed through a mesh screen or series of mesh screens to obtain the desired particle size.

A capsule may be prepared, e.g., by placing the bulk blend niraparib formulation, described above, inside of a capsule. In some embodiments, the niraparib formulations (non-aqueous suspensions and solutions) are placed in a soft gelatin capsule. In other embodiments, the niraparib formulations are placed in standard gelatin capsules or non-gelatin capsules. In other embodiments, the niraparib formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the niraparib formulation is delivered in a capsule form. For example, the capsule may comprise between about 1 mg to about 1000 mg of niraparib or a pharmaceutically acceptable salt thereof. In some embodiments, the capsule comprises from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, or 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg of niraparib or a pharmaceutically acceptable salt thereof. In some embodiments, the capsule comprises from about 1 to about 300 mg of niraparib or a pharmaceutically acceptable salt thereof. In some embodiments, the capsule comprises from about 300 mg to about 1000 mg of niraparib or a pharmaceutically acceptable salt thereof. In some embodiments, the capsule comprises about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg of niraparib or a pharmaceutically acceptable salt thereof.

Another embodiment of the present invention also provides a process for the preparation of a pharmaceutical composition of niraparib or a pharmaceutically acceptable salt thereof (e.g., niraparib tosylate monohydrate), comprising the steps of obtaining niraparib that has been screened; obtaining lactose monohydrate that has been screened with a screen; combining the screened niraparib with the screened lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. The method can further comprise encapsulating the composition comprising niraparib, lactose monohydrate and magnesium stearate.

Another embodiment of the present invention also provides a process for the preparation of a pharmaceutical composition of niraparib or a pharmaceutically acceptable salt thereof (e.g., niraparib tosylate monohydrate), comprising the steps of obtaining niraparib that has been screened with a screen having a mesh size of greater than about 425 microns; combining the screened niraparib with lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. The method can further comprise encapsulating the composition comprising niraparib, lactose monohydrate and magnesium stearate.

Another embodiment of the present invention also provides a process for the preparation of a pharmaceutical composition of niraparib or a pharmaceutically acceptable salt thereof (e.g., niraparib tosylate monohydrate), comprising the steps of obtaining niraparib that has been screened; combining the screened niraparib with the screened lactose monohydrate to form a composition comprising niraparib and lactose monohydrate, blending the composition comprising niraparib and lactose monohydrate, combining the blended composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than about 250 microns, and blending the composition comprising niraparib, lactose monohydrate and magnesium stearate.

In some embodiments, obtaining niraparib that has been screened comprises obtaining niraparib that has been screened with a screen having a mesh size of greater than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining niraparib that has been screened comprises obtaining niraparib that has been screened with a screen having a mesh size of greater than 425 μm.

In some embodiments, obtaining niraparib that has been screened comprises obtaining niraparib that has been screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining niraparib that has been screened comprises obtaining niraparib that has been screened with a screen having a mesh size of about 1180 microns.

In some embodiments, obtaining screened lactose monohydrate that has been screened with a screen comprises obtaining screened lactose monohydrate that has been screened with a screen having a mesh size of at most about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining screened lactose monohydrate that has been screened with a screen comprises obtaining screened lactose monohydrate that has been screened with a screen having a mesh size of at most about 600 microns.

In some embodiments, obtaining screened lactose monohydrate that has been screened with a screen comprises obtaining screened lactose monohydrate that has been screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining screened lactose monohydrate that has been screened with a screen comprises obtaining screened lactose monohydrate that has been screened with a screen having a mesh size of about 600 microns. In some embodiments, over 50% of the screened lactose monohydrate is present as particles with a diameter of between 53 microns and 500 microns.

In some embodiments, the magnesium stearate is magnesium stearate screened with a screen having a mesh size of greater than about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, the magnesium stearate is magnesium stearate screened with a screen having a mesh size of greater than 250 microns.

In some embodiments, the magnesium stearate is magnesium stearate screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, the magnesium stearate is magnesium stearate screened with a screen having a mesh size of about 600 microns.

In some embodiments, the method further comprises obtaining lactose monohydrate that has been screened before combining the screened niraparib with the screened lactose monohydrate to form a composition comprising niraparib and lactose monohydrate. In some embodiments, the particle size of the lactose monohydrate is about the same as the particle size of the niraparib.

In some embodiments, the composition comprising niraparib and lactose monohydrate is screened with a screen having a mesh size of at most about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, the composition comprising niraparib and lactose monohydrate is screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, the screened niraparib is screened with a conical mill, a vibratory sifter, or an oscillating screen.

In some embodiments, the method further comprises encapsulating the blended the composition comprising niraparib, lactose monohydrate and magnesium stearate.

In some embodiments, the encapsulating comprises encapsulating the blended the composition comprising niraparib, lactose monohydrate and magnesium stearate into a capsule comprising gelatin.

In some embodiments, the number of blending revolutions for blending niraparib and an excipient is about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

In some embodiments, the number of blending revolutions for blending niraparib and lactose monohydrate is about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

In some embodiments, the number of blending revolutions for blending a composition comprising niraparib and lactose monohydrate with magnesium stearate is about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

### Dose-to-Dose Uniformity

Typical capsules are packaged and administered orally. For example, a single administration (i.e. a single dose) of a niraparib capsule may include a single capsule, two capsules, three capsules or more taken orally by the subject.

The present disclosure further recognizes the challenges present in the formulation of capsules, wherein each contains substantially similar concentrations of niraparib or its pharmaceutically acceptable salts. In particular, it is desirable to achieve dose-to-dose uniformity in each capsule in term of niraparib content and/or distribution.

Dose to dose variability can be a challenge. Specifically, it is not desirable for one or more capsules of a lot or batch of capsules to have significant variations of drug content from one capsule to another. For example, it is not desirable for one or more capsules of a lot or batch of capsules encapsulated at later times during the encapsulation process to include higher concentrations of niraparib than one or more or all of the capsules encapsulated during the earlier times during the encapsulation process. It is not desirable for one or more capsules of a lot or batch of capsules encapsulated at certain times during the encapsulation process to include higher concentrations of niraparib than one or more or all of the capsules encapsulated during other times during the encapsulation process.

Without being limited as to theory, there are at least two possibilities that could result in the variations of drug content from one capsule to another. Variation could result from niraparib segregation in the bulk container or result from niraparib segregation during the encapsulation process itself. Segregation of a physical blend can occur for many reasons, but typically involves two main and sometimes co-contributing attributes: the physical properties of the formulation components and the process of manufacturing.

In some embodiments, the composition has a dose-to-dose niraparib concentration variation of less than about 50%. In some embodiments, the composition has a dose-to-dose niraparib concentration variation of less than about 40%. In some embodiments, the composition has a dose-to-dose niraparib concentration variation of less than about 30%. In some embodiments, the composition has a dose-to-dose niraparib concentration variation of less than about 20%. In some embodiments, the composition has a dose-to-dose niraparib concentration variation of less than about 10%. In some embodiments, the composition has a dose-to-dose niraparib concentration variation of less than 5%.

In some embodiments, the dose-to-dose niraparib concentration variation is based on 10 consecutive doses. In some embodiments, the dose-to-dose niraparib concentration variation is based on 8 consecutive doses. In some embodiments, the dose-to-dose niraparib concentration variation is based on 5 consecutive doses. In some embodiments, the dose-to-dose niraparib concentration variation is based on 3 consecutive doses. In some embodiments, the dose-to-dose niraparib concentration variation is based on 2 consecutive doses.

### Kits/Articles of Manufacture

If desired, the niraparib may be provided in a kit. The kits include a therapeutically effective dose of niraparib for treating diseases and conditions, such as cancer. The dosage forms may be packaged on blister cards for daily administration convenience and to improve adherence.

The disclosure also provides kits for preventing, treating or ameliorating the symptoms of a disease or disorder in a mammal. Such kits generally will comprise one or more of niraparib compositions or devices disclosed herein, and instructions for using the kit. The disclosure also contemplates the use of one or more of niraparib compositions, in the manufacture of medicaments for treating, abating, reducing, or ameliorating the symptoms of a disease, dysfunction, or disorder in a mammal, such as a human that has, is suspected of having, or at risk for developing cancer.

In some embodiments, a kit includes one or more additional containers, each with one or more of various materials (such as reagents, optionally in concentrated form, and/or devices) desirable from a commercial and user standpoint for use of a formulation described herein. Non-limiting examples of such materials include, but not limited to, buffers, diluents, filters, needles, syringes; carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the pharmaceutical compositions are presented in a pack or dispenser device which contains one or more unit dosage forms containing a compound provided herein. In another embodiment, the pack for example contains metal or plastic foil, such as a blister pack. In a further embodiment, the pack or dispenser device is accompanied by instructions for administration. In yet a further embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a compound provided herein formulated in a compatible pharmaceutical carrier are also prepared, placed in an appropriate container, and labeled for treatment of an indicated condition.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed with invention as defined in the claims which follow. The invention disclosed herein is further illustrated by the following examples which in no way should be construed as being limiting.

### Example 1

Different batches of niraparib 100 mg capsules with various batch sizes were generated by the processes described herein. The batch size ranged from about 10,000 capsules to about 300,000 capsules using V-blenders or double cone blenders. For all batches, all components (API, lactose, and magnesium stearate) were screened. Both manual and automated encapsulators were used.

Different batches produced herein are summarized in Table 1.

TABLE 1
Batches of 100 mg niraparib capsules produced

| Batch | Batch Size | | | |
|---|---|---|---|---|
| Number | (capsules) | Screening Process | Blender | Encapsulator |
| A | 108,000 | API - screened with a | double cone | manual |
| | | mesh screen | | encapsulator |
| M | 115,000 | Lactose - screened or | double cone | automated |
| B | 250,000 | used a round separator | V-blender | encapsulator |
| C | 185,000 | Magnesium stearate- | V-blender | |
| H | 18,750 | screened with mesh | V-blender | |
| I | 55,000 | screen | V-blender | |

### Example 2

A blend uniformity test was performed on a bulk hold drum at two time points. The samples were taken from the top, middle, and bottom of the drum. The results of the uniformity test are summarized in Table 2. It can be seen that the results in the % recovery column range over 5.9% for the three samples taken.

TABLE 2
Blend uniformity results of bulk hold drum

| Sample location | Sample weight (mg) | % Recovery |
|---|---|---|
| Top | 884.45 | 100.9 |
| Middle | 821.17 | 98.7 |
| Bottom | 504.30 | 95.0 |
| Average | NA | 98.2 |
| Standard Deviation | NA | 2.98 |

### Example 3

Assay and uniformity testing are described in Table 3.

TABLE 3
Assay and content uniformity of two batches

| | Assay | |
|---|---|---|
| Batch Number | (% Label Claim) | Content Uniformity |
| A | 98.0 | 6.3 |
| M | 99.7 | 2.6 |

### Example 4

Two larger scale batches were produced. With the increased scale, sampling of the blended material was conducted to confirm the process parameters used resulted in a uniform blend. The additional sampling included blend uniformity in the V-blender and in the bulk receiving container.

Bulk density and tapped density were measured and used to calculate the Hausner Ratio and Carr Index. The resultant data demonstrate a bulk density of 0.525-0.590 g/cc, a tapped density of 0.820-0.900 g/cc, a Hausner's ratio of 1.52-1.67 and a Carr's index of 34-40. Prelubrication blend uniformity after addition of magnesium stearate was uniform

### Example 5

After the blending and sampling steps, the bulk blend for batches B and C were each separated into several containers and sampled for blend uniformity before encapsulation. All containers demonstrate a similar uniformity around 100% with a low standard deviation. Both batches exhibited similar dissolution profiles.

### Example 6

Blend uniformity was taken after initial blending and after the lubricant was added. The discharged blend was then tested in the bulk container for uniformity. Encapsulation was cutoff at a pre-specified point to ensure uniform assay in capsules during the encapsulation run. Figure TA and 1B illustrate the basic manufacturing process. The blend was uniformly blended both before and after the lubricant was added. The contents were discharged into a single container for both batches to prepare for encapsulation. The single container was sampled for uniformity and results indicated that the bulk blend was uniform after transferring to the final bulk container. Bulk density and tapped density were measured and used to calculate the Hausner Ratio and Carr Index. Bulk density and tapped density were measured and used to calculate the Hausner Ratio and Carr Index. The resultant data demonstrate a bulk density of 0.516-0.582 g/cc, a tapped density of 0.831-0.0.846 g/cc, a Hausner's ratio of 1.43-1.64, a Carr's index of 20-22, and a Flowdex of 20-22 mm.

### Example 7

In preparing certain drug product batches, segregation of the blend occurred during capsule filling, particularly during the end of the filling of the powder blend. Therefore, measurement of the stratified content uniformity (SCU) of the capsules and sampling from the dosing bowl were performed at the end of the run. Sampling results demonstrated that the niraparib content throughout the setup and encapsulation was uniform. The niraparib content from the stratified content uniformity (SCU) measurements was from 98.7% to 105.6% throughout the setup and encapsulation. Results from the dosing bowl at the end of the run demonstrated a slightly higher niraparib content as compared to the bulk container blend uniformity test results (104.9% to 105.1%). The dissolution of these batches was uniform.

### Example 8

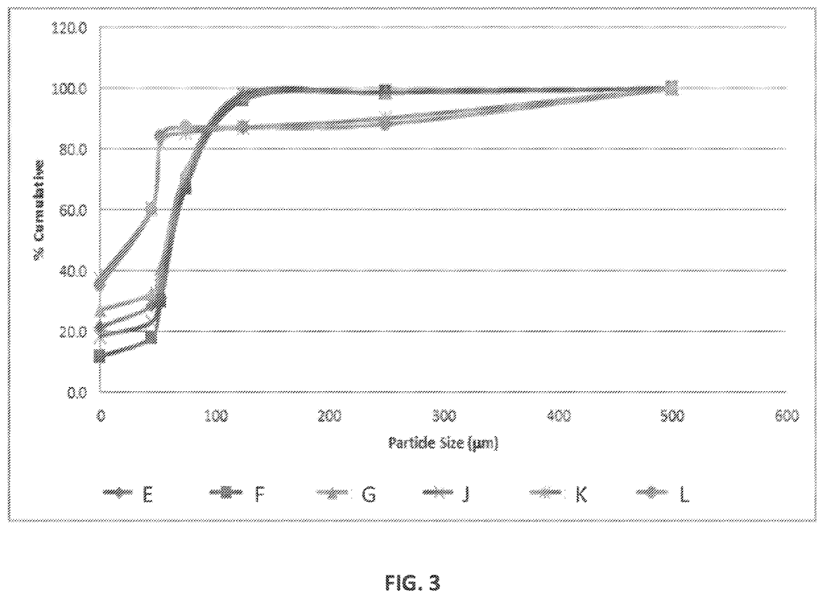

One or more batches were produced at the 185,000 capsule scale using a V-blender and an automated encapsulator. In-process sampling was performed to evaluate the uniformity of the capsules throughout the encapsulation process. Not less than twenty stratified content uniformity (SCU) in-process samples were taken over the encapsulation process of batch D. Blend uniformity testing was performed results demonstrated blend uniformity in the prelubrication blend and the final blend with a relatively low standard deviation at all sampling times. Powder characteristics of the powder blend were measured and calculated. The resultant data demonstrate a bulk density of 0.525-0.590 g/cc, a tapped density of 0.8086-0.900 g/cc, a Hausner's ratio of 1.41-1.67 and a Carr's index of 29-40, and a Flowdex of 20-22 mm. During the manufacture of the one or more batches, stratified content uniformity (SCU) was consistent throughout the run(s) until the later time points and in particular the last two time points (855 and 885 minutes). FIG. 3 illustrates the average, minimum, and maximum percent label claim values across the encapsulation process for a batch.

### Example 9

Additional batches were produced to minimize blend segregation. These batches were divided into sub-lots at various time intervals and each sub-lot was analyzed for content uniformity. The batches used are described in Table 4. The niraparib tosylate monohydrate had a volume mean diameter of about 34.4 microns to about 58.4 microns, a D_{(3,2)} of about 14.9 microns to about 23.4 microns, a bulk density of 0.34-0.45 g/cc, and/or a tapped density of 0.53-0.66 g/cc.

TABLE 4
Examples of batches manufactured

| Batch | Batch Size | | | |
|---|---|---|---|---|
| Number | (capsules) | Screening Process | Blender | Encapsulator |
| E | 185,000 | Drug substance - | V-blender | Automated |
| F | 185,000 | screened with mesh | | Encapsulator |
| G | 185,000 | screen | | (200 capsules/ |
| J | 55,000 | Lactose - screened | V-blender | minute) |
| K | 185,000 | or used round | V-blender | |
| L | 185,000 | separator | | |
| | | Magnesium Stearate | | |
| | | (screened with mesh) | | |

### Example 10

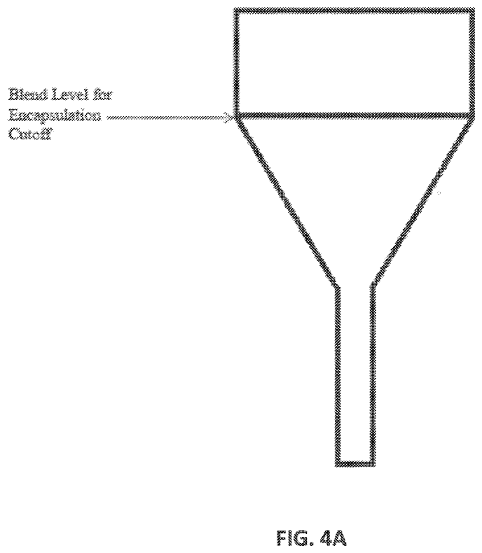

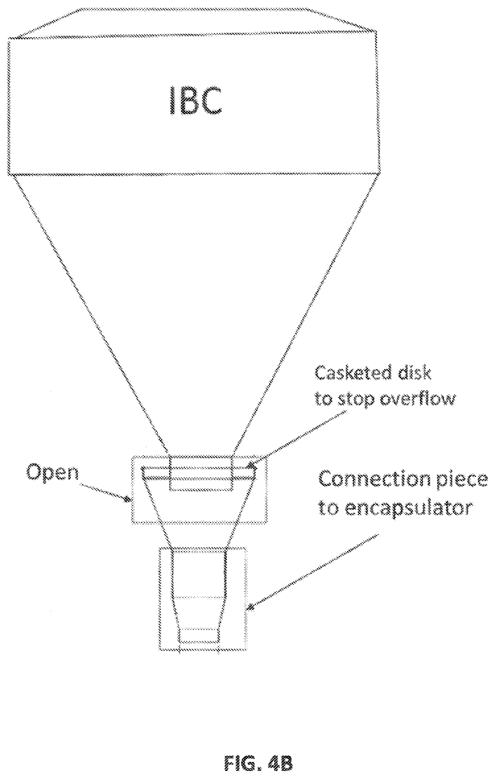

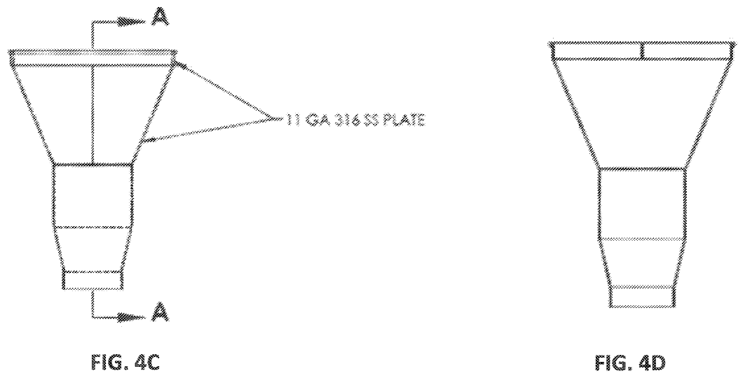

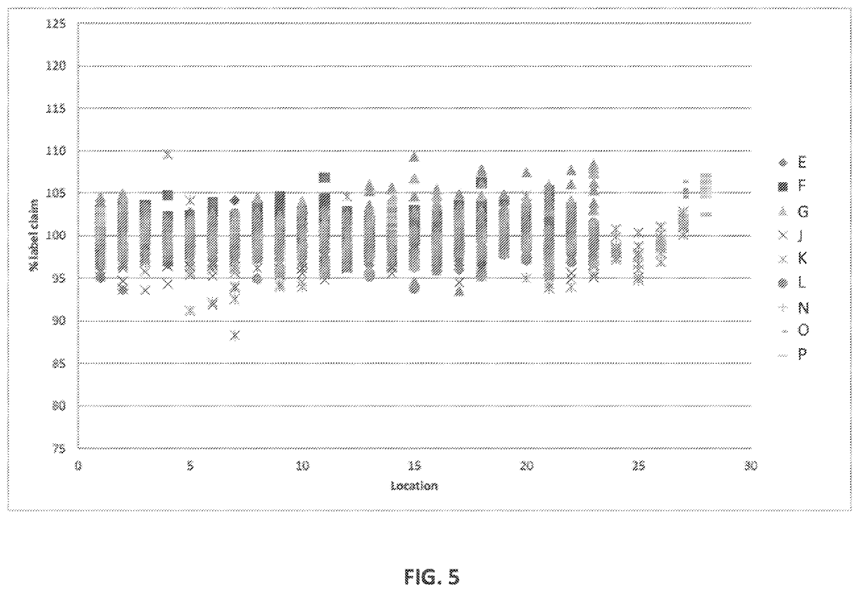

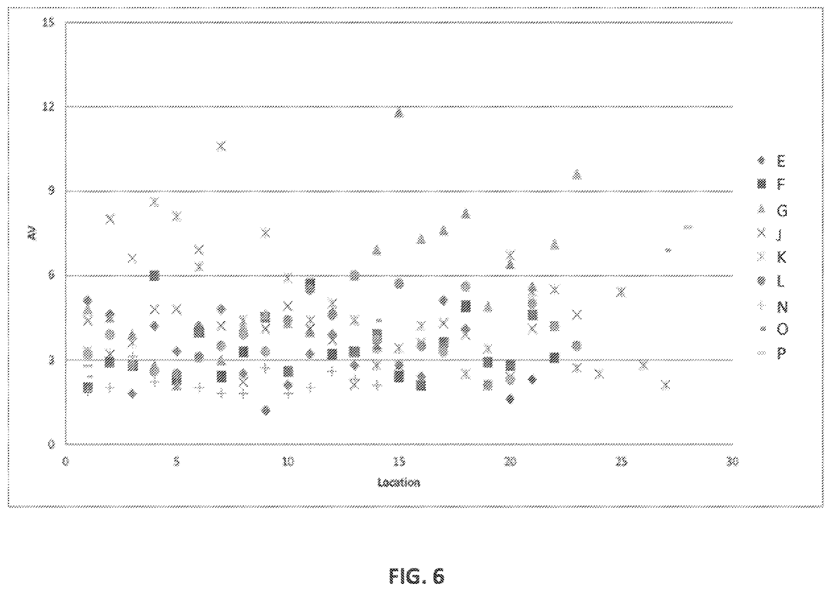

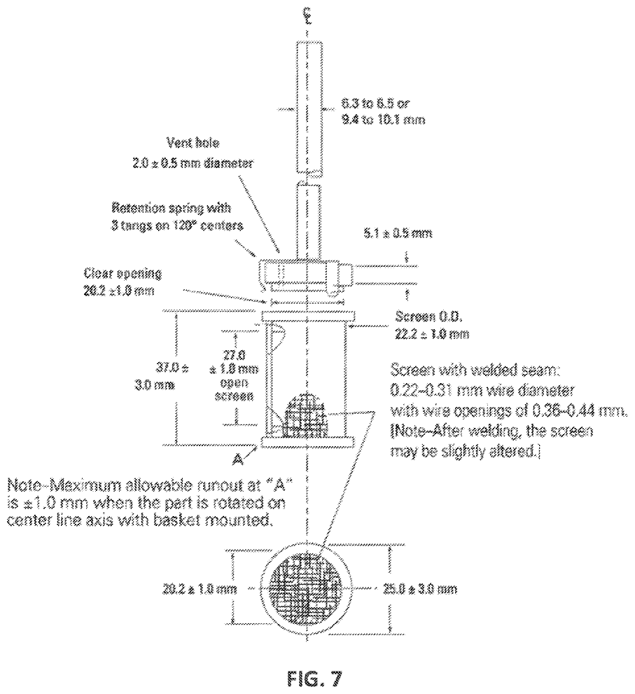

After initial mixing of the pre-lubricated blend with API and lactose (before magnesium stearate), samples were removed for blend uniformity analysis. All results demonstrated a uniform blend before the lubricant, magnesium stearate, is added. In any batch exhibiting lumps, the whole blend is removed from the V-blender, screened through a mesh screen and placed back in the V-blender for additional blending. Any changes in moisture content, if observed during blend storage, did not impact encapsulation or the final drug product. Following acceptance of the pre-lubrication blend, magnesium stearate was added and blended in V-blenders. The V-blender was sampled from various positions within the blender for final blend uniformity and the results demonstrated that the final blend was uniformly mixed. After final blending, samples are taken for analysis and demonstrate that the density of the batches were very similar. Particle size is presented graphically in FIG. 4. The final blend is discharged into bulk containers after the final blend samples are taken and show that the blend remains uniform after discharge into the bulk containers prior to encapsulation. The average % recovery for all samples taken for the batches was from 96.8% to 101.7%, indicating a reasonably uniform blend.

### Example 11

Stratified uniformity of the above sample batches was tested. To address potential segregation observed during the encapsulation, the capsules were divided into sub-lots. Once the blend hopper reached a defined level, collection of the capsules were stopped. The pre-defined cutoff point was where the powder blend reaches the end of the cylindrical portion of the blend hopper. All capsules tested prior to the cutoff passed the in-process acceptance criteria. Segregation was not observed in any of the batches.

### Example 12

Bulk hold stability was conducted on certain batches in a packaging configuration representative of commercial packaging. The capsules were tested for assay, degradation products, and dissolution at regular interval for bulk stability evaluation. Bulk hold study measurements from batches stored at 5° C., 25° C./60% RH, 30° C./65% RH, 40° C./75% RH were taken. The results demonstrated that less than 0.05% wt/wt of impurities were present initially and less than 0.05% wt/wt was present after storage for 1 and 3 months, and 0.1% after storage for 6, 9, and 12 months at 5° C., 25° C./60% RH, 30° C./65% RH, 40° C./75% RH for all samples tested. Less than or about 0.06% wt/wt of any single degradation product was present initially and less than 0.1% wt/wt of any single degradation product was present after storage for 1, 3, 6, 9, and 12 months at 5° C., 25° C./60% RH, 30° C./65% RH, 40° C./75% RH for all samples tested. Less than or about 0.06% wt/wt of total degradation product was present initially and less than 0.1% wt/wt of total degradation product was present after storage for 1, 3, 6, 9, and 12 months at 5° C., 25° C./60% RH, 30° C./65% RH, 40° C./75% RH for all samples tested. All dissolution passed the acceptance criteria.

### Example 13: Dissolution Data

100 mg niraparib capsules were manufactured. At the time of manufacture, the capsules were tested and released by USP 711 Apparatus 2 using a buffered solution. The dissolution profiles for niraparib capsules were obtained at bulk release, after packaging in the designated commercial packaging, and during stability storage at designated testing intervals. All dissolution passed the acceptance criteria.

### Example 14: Determination of Powder Composition Characteristics

Samples of powder compositions were prepared to evaluate the powder compositions disclosed herein. The following tests/measurements were made using a FT-4 powder rheometer from Freeman technology.

TABLE 5
Tests/measurements made using a FT-4 powder rheometer

| Test | Required output |
|---|---|
| Stability and | Stability profile and stability index |
| variable flow rate | Basic flowability energy |
| | Conditioned bulk density |
| | Flow rate index |
| | Specific energy |
| Wall Friction | Force vs. torque and wall friction |
| | angle, performed using extreme |
| | roughness attachments(most |
| | polished and roughest) |
| Permeability | Normal stress vs. pressure drop |
| | plots |
| Aeration | Air velocity vs. energy plots |
| | Aeration ratio |
| | Aerated energy |
| Compressibility | Normal stress vs. compressibility |
| | plots |
| | Compressibility index |
| Shear cell | Full mohr circle analysis |

The cohesion (kPa), Unconfined Yield Strength (UYS) (kPa), Maj or Principle Stress (MPS) (kPa), flow function (FF) (MPS/UYS), Angle of internal friction (AIF), and bulk density (BD) (g/cm³) were determined by carrying out shear cell tests using a FT-4 powder rheometer and the results can be seen in the tables below:

TABLE 6
Results from shear cell tests for indicated niraparib

| | Cohe- | | | | | |
|---|---|---|---|---|---|---|
| | sion, | UYS, | MPS, | | AIF, | BD, |
| Material | kPa | kPa | kPa | FF | ° | g/cm³ |
| Milled, Annealed | 0.87 | 3.32 | 17.83 | 5.37 | 34.60 | 0.33 |
| Milled, Annealed | 0.82 | 3.04 | 17.24 | 5.67 | 33.26 | 0.40 |
| Non Milled, Annealed A | 1.02 | 3.97 | 18.50 | 4.66 | 35.80 | 0.37 |
| Non Milled, Annealed A | 1.10 | 4.36 | 18.64 | 4.27 | 36.54 | 0.38 |
| Milled, Non Annealed | 1.44 | 6.09 | 20.76 | 3.41 | 39.51 | 0.82 |
| Milled, Non Annealed | 1.14 | 5.07 | 21.68 | 4.27 | 41.44 | 0.54 |
| Non Milled, Non Annealed | 2.84 | 10.46 | 19.48 | 1.86 | 32.94 | 0.53 |
| Non Milled, Non Annealed | 2.67 | 10.20 | 20.05 | 1.96 | 34.74 | 0.55 |
| Milled, Annealed | 0.75 | 2.98 | 18.81 | 6.31 | 36.91 | 0.54 |
| Milled, Annealed | 0.84 | 3.30 | 19.12 | 5.79 | 36.11 | 0.54 |
| Non Milled, Annealed | 0.65 | 2.70 | 18.87 | 6.99 | 38.33 | 0.51 |
| Non Milled, Annealed | 0.61 | 2.54 | 19.35 | 7.62 | 38.91 | 0.50 |
| Non Milled, Annealed C | 0.97 | 3.44 | 15.95 | 4.63 | 31.07 | 0.50 |
| Non Milled, Annealed C | 0.98 | 3.44 | 15.66 | 4.56 | 30.37 | 0.50 |
| Non Milled, Annealed D | 1.14 | 3.99 | 16.44 | 4.12 | 30.49 | 0.44 |
| Non Milled, Annealed D | 1.06 | 3.76 | 16.24 | 4.32 | 31.30 | 0.46 |
| Non Milled, Annealed B | 1.26 | 4.56 | 16.70 | 3.66 | 31.99 | 0.50 |
| Non Milled, Annealed B | 1.13 | 4.10 | 16.62 | 4.05 | 32.24 | 0.50 |

AIF = Angle of internal friction;
BD = bulk density;
UYS = Unconfined Yield Strength;
MPS = Major Principle Stress;
FF = flow function (MPS/UYS)

TABLE 7
Results from shear cell tests for the blends
made with the indicated niraparib

| | Cohe- | | | | | |
|---|---|---|---|---|---|---|
| | sion, | UYS, | MPS, | | AIF, | BD, |
| Material | kPa | kPa | kPa | FF | ° | g/cm³ |
| Non Milled, Annealed | 0.37 | 1.34 | 14.99 | 11.15 | 32.49 | 0.59 |
| Non Milled, Annealed | 0.32 | 1.15 | 14.61 | 12.67 | 31.43 | 0.57 |
| Milled, Annealed | 0.19 | 0.67 | 13.82 | 20.63 | 30.52 | 0.63 |
| Milled, Annealed | 0.21 | 0.73 | 14.27 | 19.45 | 30.55 | 0.65 |
| Milled, Annealed | 0.51 | 1.91 | 15.46 | 8.11 | 33.71 | 0.50 |
| Milled, Annealed | 0.41 | 1.56 | 15.49 | 9.96 | 34.98 | 0.52 |
| Non Milled, Annealed A | 0.40 | 1.54 | 15.64 | 10.14 | 35.25 | 0.49 |
| Non Milled, Annealed A | 0.32 | 1.27 | 15.61 | 12.32 | 36.25 | 0.51 |
| Non Milled, Non Annealed | 0.72 | 2.80 | 16.73 | 5.98 | 35.31 | 0.62 |
| Non Milled, Non Annealed | 0.75 | 2.86 | 16.89 | 5.91 | 34.53 | 0.61 |
| Milled, Non Annealed | 0.33 | 1.32 | 16.29 | 12.34 | 36.29 | 0.59 |
| Milled, Non Annealed | 0.56 | 2.17 | 16.27 | 7.50 | 35.00 | 0.60 |
| Non Milled, Annealed B | 0.58 | 2.18 | 14.99 | 6.88 | 33.93 | 0.59 |
| Non Milled, Annealed B | 0.57 | 2.17 | 15.11 | 6.97 | 34.60 | 0.60 |
| Non Milled, Annealed C | 0.55 | 2.05 | 14.94 | 7.28 | 33.38 | 0.61 |
| Non Milled, Annealed C | 0.32 | 1.16 | 14.41 | 12.40 | 32.84 | 0.62 |
| Non Milled, Annealed D | 0.37 | 1.34 | 14.36 | 10.69 | 32.49 | 0.58 |
| Non Milled, Annealed D | 0.27 | 1.01 | 14.51 | 14.35 | 33.85 | 0.58 |

AIF = Angle of internal friction;
BD = bulk density;
UYS = Unconfined Yield Strength;
MPS = Major Principle Stress;
FF = flow function (MPS/UYS)

### Example 15: Wall Friction Tests

A wall friction test method was developed to assess the interaction between the drug substance and stainless steel. The apparatus used is a FT-4 powder rheometer from Freeman technology. Various niraparib particles and niraparib blends obtained by the processes of the present invention were placed in a vessel containing the sample and a wall friction head to induce both vertical and rotational stresses. The powder sample was prepared by conditioning and then pre-consolidation using the standard FT4 blade and vented piston.

The wall friction head equipped with 1.2 microns average roughness of 316 Stainless Steel discs moves downwards to the surface of the sample and induces a normal stress as the disc contacts the top of the sample. The head continues to move downwards until the required normal stress is established. Slow rotation of the wall friction head then begins, inducing a shear stress. A shear plane is established between the disc and sample surfaces. As the powder bed resists the rotation of the wall friction head, the torque increases until the resistance is eventually overcome. At this point, a maximum torque is observed. The wall friction head continues to rotate at 18 degrees/min for 5 minutes. The torque required to maintain this rotational is measured which enables a “steady-state” shear stress to be calculated. The normal stress is maintained constant at the target applied stress for each step throughout that step. A series of shear stress values is measured for a range of target applied stresses. Due to the nature of the samples and the fact that an exact constant rotational torque is unlikely to be achieved, the software determines an average value during 10% of the shearing time. The wall friction angleisthencalculatedbydrawingabestfitlinethroughthedatapointson the graph, and measuring the angle subtended between this best fit line and the horizontal. The results were plotted. These results suggest that the particles of the invention exhibit less sticky behavior to metal surfaces and have thus improved processability, e.g., for automated encapsulation of niraparib formulations described herein.

TABLE 8
Results from wall friction tests for the indicated niraparib batches

| Material | Ra | WFA, ° | BD, g/cm³ |
|---|---|---|---|
| Non Milled, Annealed | 0.05 | 24.32 | 0.51 |
| Non Milled, Annealed | 0.05 | 22.60 | 0.50 |
| Non Milled, Annealed | 0.05 | 21.91 | 0.49 |
| Milled, Annealed | 0.05 | 25.26 | 0.33 |
| Milled, Annealed | 0.05 | 29.53 | 0.65 |
| Milled, Annealed | 0.05 | 28.57 | 0.33 |
| Non Milled, Annealed A | 0.05 | 0.56 | 0.37 |
| Non Milled, Annealed A | 0.05 | 25.19 | 0.38 |
| Non Milled, Annealed A | 0.05 | 33.40 | 0.39 |
| Non Milled, Non Annealed | 0.05 | 37.05 | 0.53 |
| Non Milled, Non Annealed | 0.05 | 38.17 | 0.55 |
| Non Milled, Non Annealed | 0.05 | 38.86 | −0.73 |
| Milled, Non Annealed | 0.05 | 32.16 | 0.48 |
| Milled, Non Annealed | 0.05 | 34.29 | 0.51 |
| Milled, Non Annealed | 0.05 | 31.26 | 0.50 |
| Milled, Annealed | 0.05 | 15.77 | 0.53 |
| Milled, Annealed | 0.05 | 17.30 | 0.54 |
| Milled, Annealed | 0.05 | 19.94 | 0.53 |
| Non Milled Annealed B | 0.05 | 16.71 | 0.50 |
| Non Milled Annealed B | 0.05 | 29.20 | 0.49 |
| Non Milled Annealed B | 0.05 | 30.86 | 0.48 |
| Non Milled Annealed C | 0.05 | 29.60 | 0.50 |
| Non Milled Annealed C | 0.05 | 29.83 | 0.50 |
| Non Milled Annealed C | 0.05 | 30.54 | 0.49 |
| Non Milled Annealed D | 0.05 | 27.29 | 0.44 |
| Non Milled Annealed D | 0.05 | 31.10 | 0.46 |
| Non Milled Annealed D | 0.05 | 30.98 | 0.45 |

WFA = Wall friction angle;
BD = bulk density

TABLE 9
Results from wall friction tests for powder
blends made with indicated niraparib batches.

| Material | Ra | WFA, ° | BD, g/cm³ |
|---|---|---|---|
| Non Milled, Annealed B | 0.05 | 8.15 | 0.59 |
| Non Milled, Annealed B | 0.05 | 14.09 | 0.60 |
| Non Milled, Annealed B | 0.05 | 11.63 | 0.59 |
| Non Milled, Annealed B | 1.2 | 24.39 | 0.59 |
| Non Milled, Annealed B | 1.2 | 24.25 | 0.59 |
| Non Milled, Annealed B | 1.2 | 24.15 | 0.61 |
| Non Milled, Annealed C | 0.05 | 11.00 | 0.58 |
| Non Milled, Annealed C | 0.05 | 13.05 | 0.63 |
| Non Milled, Annealed C | 0.05 | 15.52 | 0.62 |
| Non Milled, Annealed C | 1.2 | 25.21 | 0.62 |
| Non Milled, Annealed C | 1.2 | 25.72 | 0.63 |
| Non Milled, Annealed C | 1.2 | 24.38 | 0.62 |
| Milled, Annealed | 0.05 | 8.79 | 0.65 |
| Milled, Annealed | 0.05 | 17.36 | 0.65 |
| Milled, Annealed | 1.2 | 24.03 | 0.66 |
| Milled, Annealed | 1.2 | 25.02 | 0.65 |
| Non Milled, Annealed | 0.05 | 13.22 | 0.64 |
| Non Milled, Annealed | 0.05 | 16.37 | 0.63 |
| Non Milled, Annealed | 1.2 | 24.80 | 0.62 |
| Non Milled, Annealed | 1.2 | 24.70 | 0.63 |
| Milled, Annealed | 0.05 | 19.00 | 0.51 |
| Milled, Annealed | 0.05 | 22.77 | 0.54 |
| Milled, Annealed | 1.2 | 26.65 | 0.50 |
| Milled, Annealed | 1.2 | 27.23 | 0.87 |
| Non Milled, Annealed | 0.05 | 14.17 | 0.49 |
| Non Milled, Annealed | 0.05 | 22.72 | 0.52 |
| Non Milled, Annealed | 1.2 | 26.96 | 0.50 |
| Non Milled, Annealed | 1.2 | 27.78 | 0.54 |
| Non Milled, Non Annealed | 0.05 | 15.90 | 0.61 |
| Non Milled, Non Annealed | 0.05 | 21.46 | 0.62 |
| Non Milled, Non Annealed | 1.2 | 25.27 | 0.60 |
| Non Milled, Non Annealed | 1.2 | 25.57 | 0.59 |
| Milled, Non Annealed | 0.05 | 13.40 | 0.60 |
| Milled, Non Annealed | 0.05 | 15.66 | 0.60 |
| Milled, Non Annealed | 1.2 | 27.17 | 0.60 |
| Milled, Non Annealed | 1.2 | 26.86 | 0.61 |

WFA = Wall friction angle;
BD = bulk density

TABLE 10
Results from wall friction tests for smooth finish powder
blends made with indicated niraparib batches.

| Series Name | Ra | WFA, ° | BD, g/cm³ |
|---|---|---|---|
| Milled, Annealed | 0.05 | 8.79 | 0.65 |
| Milled, Annealed | 0.05 | 17.21 | 0.64 |
| Milled, Annealed | 0.05 | 17.36 | 0.65 |
| Non Milled, Non Annealed | 0.05 | 19.00 | 0.51 |
| Non Milled, Non Annealed | 0.05 | 22.77 | 0.54 |
| Non Milled, Non Annealed | 0.05 | 19.52 | 0.50 |
| Non Milled, Annealed | 0.05 | 14.17 | 0.49 |
| Non Milled, Annealed | 0.05 | 22.72 | 0.52 |
| Non Milled, Annealed | 0.05 | 18.84 | 0.53 |
| Non Milled, Non Annealed | 0.05 | 24.11 | 0.59 |
| Non Milled, Non Annealed | 0.05 | 15.90 | 0.61 |
| Non Milled, Non Annealed | 0.05 | 21.46 | 0.62 |
| Milled, Non Annealed | 0.05 | 13.40 | 0.60 |
| Milled, Non Annealed | 0.05 | 14.95 | 0.60 |
| Milled, Non Annealed | 0.05 | 15.66 | 0.60 |
| Non Milled, Annealed | 0.05 | 13.22 | 0.64 |
| Non Milled, Annealed | 0.05 | 16.37 | 0.63 |
| Non Milled, Annealed | 0.05 | 17.73 | 0.63 |

WFA = Wall friction angle;
BD = bulk density

### Example 16: Compressibility Determination

Compressibility is a measure of how density changes as a function of applied normal stress. By definition, compressibility is the percent change in volume after compression (%). The measurements were made using a FT-4 powder rheometer from Freeman technology.

Niraparib particles and blends thereof were placed in a vessel and a vented piston was used to compress the particles. The vented piston is designed such that the compression face is constructed from a woven stainless steel mesh and allows the entrained air in the powder to escape uniformly across the surface of the powder bed. A normal stress was applied in 8 sequential compression steps beginning at 0.5 kPa and ending at 15 kPa. In each step, the normal stress was held constant for 60 seconds and the compressibility was automatically calculated as a percentage change in volume. The results were plotted and the compressibility percentage measured at 15 kPa for various niraparib powder compositions.

As is illustrated by the above data in Examples 14-16, it has been found that using the methods described herein to produce powder compositions significantly increases flowability as evidenced by favorable changes in characteristics identified above, especially niraparib powders.

### Example 17: Solid Forms of Niraparib

Crystalline solid forms of niraparib can be used to prepare the formulations and capsules described herein.

Crystalline Form I of niraparib tosylate monohydrate can be prepared according to the following representative procedure. A batch of 2-{4-[(3S)-piperidin-3-yl]phenyl}-2H-indazole-7-carboxamide tosylate is dissolved in water:DMSO/200:1 to reach a concentration of about 0.15 M. The resulting mixture is heated until dissolution occurs and is then cooled to about 25° C. overnight to provide crystalline Form I of niraparib tosylate monohydrate. Crystalline Form I can be characterized by x-ray powder diffraction, differential scanning calorimetry, Raman spectroscopy, infrared spectroscopy, dynamic water vapor sorption, or any combination thereof. For example, FIG. 11 shows an exemplary X-ray powder diffraction pattern for crystalline Form I of 2-{4-[(3S)-piperidin-3-yl]phenyl}-2H-indazole-7-carboxamide.

### Paragraphs of the Embodiments

A method of making a formulation comprising niraparib comprising:
a. obtaining niraparib;
b. obtaining lactose monohydrate that has been screened with a screen;
c. combining the niraparib with the screened lactose monohydrate to form a composition comprising niraparib and lactose monohydrate;
d. blending the composition comprising niraparib and lactose monohydrate;
e. combining the blended composition comprising niraparib and lactose monohydrate with magnesium stearate to form a composition comprising niraparib, lactose monohydrate and magnesium stearate; and
f. blending the composition comprising niraparib, lactose monohydrate and magnesium stearate.

The method of paragraph [00404], wherein obtaining niraparib comprises obtaining niraparib that has been screened.

The method of paragraph [00404], wherein combining the niraparib with the screened lactose monohydrate comprises combining unscreened niraparib with the screened lactose monohydrate.

A method of making a formulation comprising niraparib comprising:
a. obtaining niraparib, wherein the niraparib is optionally niraparib that has been screened;
b. obtaining lactose monohydrate that has been screened with a screen;
c. combining the screened niraparib with the screened lactose monohydrate to form a composition comprising niraparib and lactose monohydrate;
d. blending the composition comprising niraparib and lactose monohydrate;
e. combining the blended composition comprising niraparib and lactose monohydrate with magnesium stearate to form a composition comprising niraparib, lactose monohydrate and magnesium stearate; and
f. blending the composition comprising niraparib, lactose monohydrate and magnesium stearate.

The method of paragraph [00407], wherein obtaining niraparib comprises obtaining niraparib that has been screened.

The method of paragraph [00408], wherein obtaining niraparib that has been screened comprises obtaining niraparib that has been screened with a screen having a mesh size of greater than about 425 microns.

The method of paragraph [00409], wherein obtaining niraparib that has been screened with a screen having a mesh size of greater than about 425 microns comprises obtaining niraparib that has been screened with a screen having a mesh size of about 850 microns or about 1180 microns.

The method of any one of paragraphs [00404]-[00410], wherein obtaining lactose monohydrate that has been screened with a screen comprises obtaining screened lactose monohydrate that has been screened with a screen having a mesh size of at most about 600 microns.

The method of paragraph [00411], wherein over 50% of the screened lactose monohydrate is present as particles with a diameter of between about 53 microns and about 500 microns.

The method of any one of paragraphs [00404]-[00412], wherein the magnesium stearate is magnesium stearate screened with a screen having a mesh size of greater than about 250 microns.

The method of paragraph [00413], wherein the magnesium stearate is magnesium stearate screened with a screen having a mesh size of about 600 microns.

The method of any one of paragraphs [00404]-[00414], wherein the method further comprises screening the blended composition comprising niraparib and lactose monohydrate before combining the blended composition comprising niraparib and lactose monohydrate with magnesium stearate.

The method of paragraph [00415], wherein the blended composition comprising niraparib and lactose monohydrate is screened with a screen having a mesh size of about 600 microns.

A method of making a formulation comprising niraparib comprising:
a. obtaining niraparib, wherein the niraparib is optionally niraparib that has been screened with a screen having a mesh size of greater than about 425 microns;
b. combining the niraparib with lactose monohydrate to form a composition comprising niraparib and lactose monohydrate;
c. blending the composition comprising niraparib and lactose monohydrate;
d. combining the blended composition comprising niraparib and lactose monohydrate with magnesium stearate to form a composition comprising niraparib, lactose monohydrate and magnesium stearate; and
e. blending the composition comprising niraparib, lactose monohydrate and magnesium stearate.

The method of paragraph [00417], wherein the lactose monohydrate has been screened before combining the screened niraparib with the lactose monohydrate to form a composition comprising niraparib and lactose monohydrate.

The method of paragraph [00418], wherein the lactose monohydrate that has been screened has been screened with a screen having a mesh size of at most about 600 microns.

The method of paragraph [00418] or [00419], wherein over 50% of the screened lactose monohydrate is present as particles with a diameter of between about 53 microns and 500 microns.

The method of any one of paragraphs [00417]-[00420], wherein obtaining niraparib that has been screened with a screen having a mesh size of greater than about 425 microns comprises obtaining niraparib that has been screened with a screen having a mesh size of about 850 microns or about 1180 microns.

The method of any one of paragraphs [00417]-[00421], wherein the magnesium stearate is magnesium stearate screened with a screen having a mesh size of greater than about 250 microns.

The method of paragraph [00422], wherein the magnesium stearate is magnesium stearate screened with a screen having a mesh size of about 600 microns.

The method of any one of paragraphs [00417]-[00423], wherein the method further comprises screening the blended composition comprising niraparib and lactose monohydrate before combining the blended composition comprising niraparib and lactose monohydrate with magnesium stearate.

The method of paragraph [00424], wherein the blended composition comprising niraparib and lactose monohydrate is screened with a screen having a mesh size of about 600 microns.

A method of making a formulation comprising niraparib comprising:
a. obtaining niraparib, wherein optionally niraparib is niraparib that has been screened;
b. combining the niraparib with lactose monohydrate to form a composition comprising niraparib and lactose monohydrate,
c. blending the composition comprising niraparib and lactose monohydrate,
d. combining the blended composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than about 250 microns, and
e. blending the composition comprising niraparib, lactose monohydrate and magnesium stearate.

The method of paragraph [00426], wherein the magnesium stearate is magnesium stearate screened with a screen having a mesh size of about 600 microns.

The method of paragraph [00426] or [00427], wherein the lactose monohydrate has been screened before combining the screened niraparib with the lactose monohydrate to form a composition comprising niraparib and lactose monohydrate.

The method of paragraph [00428], wherein the lactose monohydrate has been screened with a screen having a mesh size of at most about 600 microns.

The method of paragraph [00428] or [00429], wherein over 50% of the screened lactose monohydrate is present as particles with a diameter of between about 53 microns and 500 microns.

The method of any one of paragraphs [00426]-[00430], wherein obtaining niraparib that has been screened comprises obtaining niraparib that has been screened with a screen having a mesh size of greater than about 425 microns.

The method of paragraph [00431], wherein obtaining niraparib that has been screened with a screen having a mesh size of greater than about 425 microns comprises obtaining niraparib that has been screened with a screen having a mesh size of about 850 microns or about 1180 microns.

The method of any one of paragraphs [00426]-[00432], wherein the method further comprises screening the blended composition comprising niraparib and lactose monohydrate before combining the blended composition comprising niraparib and lactose monohydrate with magnesium stearate.

The method of paragraph [00433], wherein the blended composition comprising niraparib and lactose monohydrate is screened with a screen having a mesh size of about 600 microns.

A method of making a formulation comprising niraparib comprising:
a. obtaining niraparib, wherein optionally niraparib is niraparib that has been screened;
b. combining the niraparib with lactose monohydrate to form a composition comprising niraparib and lactose monohydrate;
c. blending the composition comprising niraparib and lactose monohydrate;
d. screening the blended composition comprising niraparib and lactose monohydrate;
e. combining the screened composition comprising niraparib and lactose monohydrate with magnesium stearate to form a composition comprising niraparib, lactose monohydrate and magnesium stearate; and
f. blending the composition comprising niraparib, lactose monohydrate and magnesium stearate.

The method of paragraph [00435], wherein the blended composition comprising niraparib and lactose monohydrate is screened with a screen having a mesh size of about 600 microns.

The method of paragraph [00435] or [00436], wherein the lactose monohydrate has been screened before combining the screened niraparib with the lactose monohydrate to form a composition comprising niraparib and lactose monohydrate.

The method of paragraph [00437], wherein the lactose monohydrate has been screened with a screen having a mesh size of at most about 600 microns.

The method of paragraph [00437] or [00438], wherein over 50% of the screened lactose monohydrate is present as particles with a diameter of between about 53 microns and 500 microns.

The method of any one of paragraphs [00435]-[00439], wherein obtaining niraparib that has been screened comprises obtaining niraparib that has been screened with a screen having a mesh size of greater than about 425 microns.

The method of paragraph [00440], wherein obtaining niraparib that has been screened with a screen having a mesh size of greater than about 425 microns comprises obtaining niraparib that has been screened with a screen having a mesh size of about 850 microns or about 1180 microns.

The method of any one of paragraphs [00435]-[00441], wherein the magnesium stearate is magnesium stearate screened with a screen having a mesh size of greater than about 250 microns.

The method of paragraph [00442], wherein the magnesium stearate is magnesium stearate screened with a screen having a mesh size of about 600 microns.

The method of any one of paragraphs [00404]-[00443], wherein the screened niraparib has been annealed one or more times.

A method of making a formulation comprising niraparib comprising:
a. obtaining niraparib, wherein optionally niraparib is niraparib that has been screened, wherein the niraparib has been annealed two or more times;
b. combining the niraparib with lactose monohydrate to form a composition comprising niraparib and lactose monohydrate;
c. blending the composition comprising niraparib and lactose monohydrate;
d. combining the blended composition comprising niraparib and lactose monohydrate with magnesium stearate to form a composition comprising niraparib, lactose monohydrate and magnesium stearate; and
e. blending the composition comprising niraparib, lactose monohydrate and magnesium stearate.

The method of paragraph [00445], wherein the blended composition comprising niraparib and lactose monohydrate is screened with a screen having a mesh size of about 600 microns.

The method of paragraph [00445] or [00446], wherein the lactose monohydrate has been screened before combining the screened niraparib with the lactose monohydrate to form a composition comprising niraparib and lactose monohydrate.

The method of paragraph [00447], wherein the lactose monohydrate has been screened with a screen having a mesh size of at most about 600 microns.

The method of paragraph [00447] or [00448], wherein over 50% of the screened lactose monohydrate is present as particles with a diameter of between about 53 microns and 500 microns.

The method of any one of paragraphs [00445]-[00449], wherein obtaining niraparib that has been screened comprises obtaining niraparib that has been screened with a screen having a mesh size of greater than about 425 microns.

The method of paragraph [00450], wherein obtaining niraparib that has been screened with a screen having a mesh size of greater than about 425 microns comprises obtaining niraparib that has been screened with a screen having a mesh size of about 850 microns or about 1180 microns.

The method of any one of paragraphs [00445]-[00451], wherein the magnesium stearate is magnesium stearate screened with a screen having a mesh size of greater than about 250 microns.

The method of paragraph [00452], wherein the magnesium stearate is magnesium stearate screened with a screen having a mesh size of about 600 microns.

The method of any one of paragraphs [00445]-[00453], wherein the method further comprises screening the blended composition comprising niraparib and lactose monohydrate before combining the blended composition comprising niraparib and lactose monohydrate with magnesium stearate.

The method of paragraph [00454], wherein the blended composition comprising niraparib and lactose monohydrate is screened with a screen having a mesh size of about 600 microns.

A method of making a formulation comprising niraparib comprising:
a. obtaining niraparib that has been screened with a screen having a mesh size of greater than about 425 microns;
b. obtaining lactose monohydrate that has been screened with a screen;
c. combining the screened niraparib with lactose monohydrate to form a composition comprising niraparib and lactose monohydrate;
d. blending the composition comprising niraparib and lactose monohydrate;
e. screening the blended composition comprising niraparib and lactose monohydrate;
f. combining the screened composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than about 250 microns; and
g. blending the composition comprising niraparib, lactose monohydrate and magnesium stearate.

The method of paragraph [00456], wherein the niraparib has been annealed one or more times.

The method of any one of paragraphs [00404]-[00457], wherein the niraparib has been milled.

The method of paragraph [00458], wherein the niraparib has been wet milled.

The method of any one of paragraphs [00404]-[00459], wherein the niraparib is screened, wherein the screening may be delumping or other such powder handling manually or mechanically.

The method of any one of paragraphs [00404]-[00460], wherein the method further comprises encapsulating the blended the composition comprising niraparib, lactose monohydrate and magnesium stearate into one or more capsules.

The method of paragraph [00461], wherein the one or more capsules are gelatin capsules.

The method of paragraph [00461] or [00462], wherein the encapsulating comprises using an encapsulator.

The method of any one of paragraphs [00461]-[00463], wherein the encapsulating comprises encapsulating at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 100,000, 150,000, 200,000, 300,000, 400,000, or 500,000 of the one or more capsules.

The method of any one of paragraphs [00461]-[00464], wherein the encapsulating comprises encapsulating at a rate of at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 75,000, 100,000, 150,000 or 200,000 of the one or more capsules/hour.

The method of any one of paragraphs [00461]-[00465], wherein the encapsulating comprises encapsulating the one or more capsules from a batch comprising the composition comprising niraparib, lactose monohydrate and magnesium stearate that is in the encapsulator.

The method of paragraph [00466], wherein a portion of the volume of the batch in the encapsulator is used to encapsulate the one or more capsules.

The method of paragraph [00467], the portion of the volume of the batch in the encapsulator used to encapsulate the one or more capsules is less than about 100%, 99%, 98%, 97%, 96%, 95%, 90%, 85%, 80%, or 75% of a total initial volume of the batch.

The method of any one of paragraphs [00461]-[00468], wherein one or more parts of the encapsulator are coated with a coating.

The method of paragraph [00469], wherein the one or more coated parts comprises a tamping pin, a dosing disc, or both.

The method of paragraph [00469] or [00470], wherein the coating comprises nickel, chrome, or a combination thereof.

The method of any one of paragraphs [00461]-[00471], wherein the encapsulating comprises automatic encapsulation.

The method of any one of paragraphs [00461]-[00472], wherein adherence of the composition to one or more encapsulating components is reduced or prevented.

The method of any one of paragraphs [00461]-[00473], wherein jamming of the encapsulator is reduced or prevented.

The method of any one of paragraphs [00404]-[00474], wherein blending the composition comprising niraparib and lactose monohydrate comprises blending for about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

The method of any one of paragraphs [00404]-[00475], wherein blending the composition comprising niraparib, lactose monohydrate and magnesium stearate comprises blending for about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

The method of any one of paragraphs [00404]-[00476], wherein the blending comprises using a blender, and wherein the niraparib is distributed with substantial uniformity throughout the blender.

The method of any one of paragraphs [00461]-[00477], wherein a dose-to-dose niraparib concentration variation in the one or more capsules is less than about 50%.

The method of paragraph [00478], wherein the dose-to-dose niraparib concentration variation in the one or more capsules is less than about 40%.

The method of paragraph [00478], wherein the dose-to-dose niraparib concentration variation in the one or more capsules is less than about 30%.

The method of paragraph [00478], wherein the dose-to-dose niraparib concentration variation in the one or more capsules is less than about 20%.

The method of paragraph [00478], wherein the dose-to-dose niraparib concentration variation in the one or more capsules is less than about 10%.

The method of paragraph [00478], wherein the dose-to-dose niraparib concentration variation in the one or more capsules is less than about 5%.

The method of any one of paragraphs [00478]-[00483], wherein the dose-to-dose niraparib concentration variation is based on 10 consecutive doses or fewer.

The method of paragraph [00484], wherein the dose-to-dose niraparib concentration variation is based on 8 consecutive doses.

The method of paragraph [00484], wherein the dose-to-dose niraparib concentration variation is based on 5 consecutive doses.

The method of paragraph [00484], wherein the dose-to-dose niraparib concentration variation is based on 3 consecutive doses.

The method of paragraph [00484], wherein the dose-to-dose niraparib concentration variation is based on 2 consecutive doses.

A formulation comprising
a. an effective amount of niraparib to inhibit polyadenosine diphosphate ribose polymerase (PARP) when administered to a human,
b. lactose monohydrate, and
c. magnesium stearate;
wherein the formulation comprising niraparib, lactose monohydrate and magnesium stearate produced according the method of any one of paragraphs [00404]-[00488].

A formulation comprising
a. an effective amount of niraparib to inhibit polyadenosine diphosphate ribose polymerase (PARP) when administered to a human,
b. lactose monohydrate, and
c. magnesium stearate.

A formulation comprising
a. an effective amount of niraparib to inhibit polyadenosine diphosphate ribose polymerase (PARP) when administered to a human,
b. lactose monohydrate, and
c. magnesium stearate;
wherein the niraparib has been annealed two or more times.

A formulation comprising
a. an effective amount of niraparib to inhibit polyadenosine diphosphate ribose polymerase (PARP) when administered to a human,
b. lactose monohydrate, and
c. magnesium stearate;
wherein the niraparib in the capsule has a Hausner's ratio of less than about 1.7.

The formulation of paragraph [00492], wherein the niraparib has a Hausner's ratio of about 1.48 or less.

The formulation of paragraph [00492], wherein the niraparib has a Hausner's ratio of about 1.38 or less.

A formulation comprising
a. an effective amount of niraparib to inhibit polyadenosine diphosphate ribose polymerase (PARP) when administered to a human,
b. lactose monohydrate, and
c. magnesium stearate;
wherein the formulation has a Hausner's ratio of about 1.7 or less.

The formulation of paragraph [00495], wherein the formulation has a Hausner's ratio of about 1.64 or less.

The formulation of paragraph [00495], wherein the formulation has a Hausner's ratio of about 1.52 or less.

The formulation of paragraph [00495], wherein the formulation has a Hausner's ratio of about 1.47 or less.

The formulation of paragraph [00495], wherein the formulation has a Hausner's ratio of about 1.43 or less.

The formulation of paragraph [00495], wherein the formulation has a Hausner's ratio of about 1.41 or less.

A formulation comprising
a. an effective amount of niraparib to inhibit polyadenosine diphosphate ribose polymerase (PARP) when administered to a human,
b. lactose monohydrate, and
c. magnesium stearate;
wherein the lactose monohydrate has (i) a bulk density of about 0.2-0.8 mg/cm³ and/or (ii) a tapped density of about 0.3-0.9 mg/cm³.

A formulation comprising
a. an effective amount of niraparib to inhibit polyadenosine diphosphate ribose polymerase (PARP) when administered to a human,
b. lactose monohydrate particles, and
c. magnesium stearate;
wherein about 50% or more of the lactose monohydrate particles has a diameter of at least about 53 microns to about 500 microns, and/or about 50% or more of the lactose monohydrate particles has a diameter of at most about 250 microns.

The formulation of any one of paragraphs [00489]-[00502], wherein the niraparib has an internal friction angle of about 33.1 degrees or higher.

The formulation of any one of paragraphs [00489]-[00503], wherein the formulation has an internal friction angle of less than about 34 degrees.

The formulation of any one of paragraphs [00489]-[00504], wherein the niraparib has a flow function ratio value of more than about 6.4.

The formulation of any one of paragraphs [00489]-[00505], wherein the formulation has a flow function ratio value of more than about 14.4.

The formulation of any one of paragraphs [00489]-[00506], wherein the niraparib has a wall friction angle of less than about 29 at an Ra of about 0.05.

The formulation of any one of paragraphs [00489]-[00507], wherein the formulation has a wall friction angle of less than about 15 degrees at an Ra of about 0.05.

The formulation of any one of paragraphs [00489]-[00508], wherein the formulation has a wall friction angle of less than about 26 degrees at an Ra of about 1.2.

The formulation of any one of paragraphs [00489]-[00509], wherein the formulation is stable with respect to niraparib degradation after storage for about 1 month, 3 months, 6 months, 9 months, 12 months, 24 months, or 36 months.

The formulation of paragraph [00510], wherein the formulation is stable with respect to niraparib degradation after storage for about 1 month, 3 months, 6 months, 9 months, 12 months, 24 months, or 36 months at 5° C.

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 5° C.

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 25° C. and about 60% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 30° C. and about 65% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 40° C. and about 75% relative humidity (RH)

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 5° C.

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 25° C. and about 60% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 30° C. and about 65% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 40° C. and about 75% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 5° C.

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 25° C. and about 60% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for 1 month, 3 months, 6 months, 9 months, 12 months, 24 months, or 36 months at about 30° C. and about 65% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 40° C. and about 75% relative humidity (RH).

The formulation of paragraph [00510], wherein the formulation comprises less than about 3.0%, 2.5%, 2.0%, 1.5% 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 5° C.

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 30° C. and about 65% relative humidity (RH).

The formulation of paragraph [00510], wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 40° C. and about 70% relative humidity (RH).

The formulation of any one of paragraphs [00489]-[00526], wherein the formulation has an absolute bioavailability of niraparib of about 60 to about 90%.

The formulation of any one of paragraphs [00489]-[00527], wherein not less than about 30%, 35%, 40%, 45%, 55%, 60%, 65% 70%, 75%, 80%, 85%, 90%, 95%, or 100% of the niraparib dissolves in about 5, 10, 15, 20, 30, 45, 60, 90, or 120 minutes under dissolution evaluation.

The formulation of paragraph [00528] or [00529], wherein not less than about 30%, 35%, 40%, 45%, 55%, 60%, 65% 70%, 75%, 80%, 85%, 90%, 95%, or 100% of the niraparib dissolves in about 5, 10, 15, 20, 30, 45, 60, 90, or 120 minutes under dissolution evaluation after storage of the composition for about 1 month, 3 months, 6 months, 9 months, 12 months, 24 months, or 36 months at about 25° C. and about 60% relative humidity (RH).

The formulation of any one of paragraphs [00489]-[00529], comprising niraparib tosylate monohydrate in an amount that is about 19.16%, 38.32%, 57.48%, or 76.64% by weight of the composition.

The formulation of any one of paragraphs [00489]-[00529], comprising niraparib tosylate monohydrate in an amount that is about 19.2 to about 38.3% w/w niraparib.

The formulation of any one of paragraphs [00489]-[00529], comprising about 50 mg to about 300 mg of niraparib tosylate monohydrate, about 100 mg to about 200 mg of niraparib tosylate monohydrate, or about 125 mg to about 175 mg of niraparib tosylate monohydrate.

The formulation of paragraph [00532], comprising about 79.7 mg, about 159.4 mg, about 318.8 mg, or about 478.2 mg niraparib tosylate monohydrate.

The formulation of any one of paragraphs [00489]-[00529], comprising about 100 mg of niraparib based on free base.

The formulation of paragraph [00534], comprising about 159.4 mg niraparib tosylate monohydrate.

The formulation of any one of paragraphs [00489]-[00535], comprising about 61.2 to about 80.3% w/w lactose monohydrate.

The formulation of any one of paragraphs [00489]-[00536], comprising at least about 0.5% w/w magnesium stearate.

A capsule comprising the formulation of any one of paragraphs [00489]-[00537].

A method of treating cancer, comprising administering to a subject in need thereof the formulation according to any one of paragraphs [00489]-[00537] or the capsule of paragraph [00538].

The method of paragraph [00539], wherein the capsule is administered in doses having a dose-to-dose niraparib concentration variation of less than 50%, less than 40%, less than 30%, less than 20%, less than 10%, or less than 5%.

The method of paragraph [00539] or [00540], wherein the cancer is selected from the group consisting of ovarian cancer, breast cancer, cervical cancer, endometrial cancer, prostate cancer, testicular cancer, pancreatic cancer, esophageal cancer, head and neck cancer, gastric cancer, bladder cancer, lung cancer, bone cancer, colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma, bladder cancer, liver cancer, kidney cancer, myeloma, lymphoma, and combinations thereof.

The method of any one of paragraphs [00539]-[00541], wherein the cancer is selected from the group consisting of ovarian cancer, fallopian tube cancer, primary peritoneal cancer, and combinations thereof.

The method of any one of paragraphs [00539]-[00542], wherein the cancer is a recurrent cancer.

The method of any one of paragraphs [00539]-[00543], wherein the subject is a human subject.

The method of paragraph [00544], wherein the human subject was previously treated with a chemotherapy.

The method of paragraph [00545], wherein a chemotherapy is a platinum-based chemotherapy.

The method of paragraph [00545] or [00546], wherein the human subject had a complete or partial response to the chemotherapy.

The method of any one of paragraphs [00539]-[00547], wherein the subject has a mean peak plasma concentration (Cmax) of 600 ng/mL to 1000 ng/mL of the niraparib.

The method of paragraph [00548], wherein the subject has the mean peak plasma concentration (Cmax) within 0.5 to 6 hours after the administering.

The method of any one of paragraphs [00539]-[00549], wherein about 60%, 65%, 70%, 75%, 80%, 85% or 90% of the niraparib is bound to human plasma protein of the subject after the administering.

The method of any one of paragraphs [00539]-[00550], wherein an apparent volume of distribution (Vd/F) of the niraparib is from about 500 L to about 2000 L after administration to a human subject.

The method of anyone of paragraphs [00539]-[00551], wherein the niraparib has a mean terminal half-life (t_{1/2}) of from about 30 to about 60 hours after the administering.

The method of any one of paragraphs [00539]-[00552], wherein the niraparib has an apparent total clearance (CL/F) of from about 10 L/hour to about 20 L/hour after the administering.

The method of any one of paragraphs [00539]-[00553], wherein at least about 60%, 65%, 70%, 75%, 80%, 85%, 90%, 95%, 96%, 97%, 98%, 99%, or 100% of the niraparib is released from the composition within 1 minute, or within 5 minutes, or within 10 minutes, or within 15 minutes, or within 30 minutes, or within 60 minutes or within 90 minutes after the administering.

The method of any one of paragraphs [00539]-[00554], wherein the subject has a Cmin niraparib blood plasma level at steady state of from about 10 ng/ml to about 100 ng/ml after the administering.

The method of any one of paragraphs [00539]-[00555], wherein at least about 70%, 80%, 90%, or 95% of the niraparib is absorbed into the bloodstream of the subject within 1, 2, 3, 4, 5, 6, 7, 8, 9, 10, 11, 12, 16, 18, or 24 hours after administering.

What is claimed is:

1. A method of making a formulation comprising niraparib comprising:
(a) obtaining niraparib;
(b) obtaining lactose monohydrate that has been screened with a screen;
(c) combining the niraparib with the screened lactose monohydrate to form a composition comprising niraparib and lactose monohydrate;
(d) blending the composition comprising niraparib and lactose monohydrate;
(e) combining the blended composition comprising niraparib and lactose monohydrate with magnesium stearate to form a composition comprising niraparib, lactose monohydrate and magnesium stearate; and
(f) blending the composition comprising niraparib, lactose monohydrate and magnesium stearate.

2. The method of claim 1, wherein obtaining niraparib comprises obtaining niraparib that has been screened.

3. The method of claim 1, wherein combining the niraparib with the screened lactose monohydrate comprises combining unscreened niraparib with the screened lactose monohydrate.

4. A method of making a formulation comprising niraparib comprising:
(a) obtaining niraparib, wherein the niraparib is optionally niraparib that has been screened;
(b) obtaining lactose monohydrate that has been screened with a screen;
(c) combining the screened niraparib with the screened lactose monohydrate to form a composition comprising niraparib and lactose monohydrate;
(d) blending the composition comprising niraparib and lactose monohydrate;
(e) combining the blended composition comprising niraparib and lactose monohydrate with magnesium stearate to form a composition comprising niraparib, lactose monohydrate and magnesium stearate; and
(f) blending the composition comprising niraparib, lactose monohydrate and magnesium stearate.

5. The method of claim 4, wherein obtaining niraparib comprises obtaining niraparib that has been screened.

6. The method of claim 5, wherein obtaining niraparib that has been screened comprises obtaining niraparib that has been screened with a screen having a mesh size of greater than about 425 microns.

7. The method of claim 6, wherein obtaining niraparib that has been screened with a screen having a mesh size of greater than about 425 microns comprises obtaining niraparib that has been screened with a screen having a mesh size of about 850 microns or about 1180 microns.

8. The method of any one of claims 1-7, wherein obtaining lactose monohydrate that has been screened with a screen comprises obtaining screened lactose monohydrate that has been screened with a screen having a mesh size of at most about 600 microns.

9. The method of claim 8, wherein over 50% of the screened lactose monohydrate is present as particles with a diameter of between about 53 microns and about 500 microns.

10. The method of any one of claims 1-9, wherein the magnesium stearate is magnesium stearate screened with a screen having a mesh size of greater than about 250 microns.

11. The method of claim 10, wherein the magnesium stearate is magnesium stearate screened with a screen having a mesh size of about 600 microns.

12. The method of any one of claims 1-11, wherein the method further comprises screening the blended composition comprising niraparib and lactose monohydrate before combining the blended composition comprising niraparib and lactose monohydrate with magnesium stearate.

13. The method of claim 12, wherein the blended composition comprising niraparib and lactose monohydrate is screened with a screen having a mesh size of about 600 microns.

14. A method of making a formulation comprising niraparib comprising:
(a) obtaining niraparib, wherein the niraparib is optionally niraparib that has been screened with a screen having a mesh size of greater than about 425 microns;
(b) combining the niraparib with lactose monohydrate to form a composition comprising niraparib and lactose monohydrate;
(c) blending the composition comprising niraparib and lactose monohydrate;
(d) combining the blended composition comprising niraparib and lactose monohydrate with magnesium stearate to form a composition comprising niraparib, lactose monohydrate and magnesium stearate; and
(e) blending the composition comprising niraparib, lactose monohydrate and magnesium stearate.

15. The method of claim 14, wherein the lactose monohydrate has been screened before combining the screened niraparib with the lactose monohydrate to form a composition comprising niraparib and lactose monohydrate.

16. The method of claim 15, wherein the lactose monohydrate that has been screened has been screened with a screen having a mesh size of at most about 600 microns.

17. The method of claim 15 or 16, wherein over 50% of the screened lactose monohydrate is present as particles with a diameter of between about 53 microns and 500 microns.

18. The method of any one of claims 14-17, wherein obtaining niraparib that has been screened with a screen having a mesh size of greater than about 425 microns comprises obtaining niraparib that has been screened with a screen having a mesh size of about 850 microns or about 1180 microns.

19. The method of any one of claims 14-18, wherein the magnesium stearate is magnesium stearate screened with a screen having a mesh size of greater than about 250 microns.

20. The method of claim 19, wherein the magnesium stearate is magnesium stearate screened with a screen having a mesh size of about 600 microns.

21. The method of any one of claims 14-20, wherein the method further comprises screening the blended composition comprising niraparib and lactose monohydrate before combining the blended composition comprising niraparib and lactose monohydrate with magnesium stearate.

22. The method of claim 21, wherein the blended composition comprising niraparib and lactose monohydrate is screened with a screen having a mesh size of about 600 microns.

23. A method of making a formulation comprising niraparib comprising:
(a) obtaining niraparib, wherein optionally niraparib is niraparib that has been screened;
(b) combining the niraparib with lactose monohydrate to form a composition comprising niraparib and lactose monohydrate,
(c) blending the composition comprising niraparib and lactose monohydrate,
(d) combining the blended composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than about 250 microns, and
(e) blending the composition comprising niraparib, lactose monohydrate and magnesium stearate.

24. The method of claim 23, wherein the magnesium stearate is magnesium stearate screened with a screen having a mesh size of about 600 microns.

25. The method of claim 23 or 24, wherein the lactose monohydrate has been screened before combining the screened niraparib with the lactose monohydrate to form a composition comprising niraparib and lactose monohydrate.

26. The method of claim 25, wherein the lactose monohydrate has been screened with a screen having a mesh size of at most about 600 microns.

27. The method of claim 25 or 26, wherein over 50% of the screened lactose monohydrate is present as particles with a diameter of between about 53 microns and 500 microns.

28. The method of any one of claims 23-27, wherein obtaining niraparib that has been screened comprises obtaining niraparib that has been screened with a screen having a mesh size of greater than about 425 microns.

29. The method of claim 28, wherein obtaining niraparib that has been screened with a screen having a mesh size of greater than about 425 microns comprises obtaining niraparib that has been screened with a screen having a mesh size of about 850 microns or about 1180 microns.

30. The method of any one of claims 23-29, wherein the method further comprises screening the blended composition comprising niraparib and lactose monohydrate before combining the blended composition comprising niraparib and lactose monohydrate with magnesium stearate.

31. The method of claim 30, wherein the blended composition comprising niraparib and lactose monohydrate is screened with a screen having a mesh size of about 600 microns.

32. A method of making a formulation comprising niraparib comprising:
(a) obtaining niraparib, wherein optionally niraparib is niraparib that has been screened;
(b) combining the niraparib with lactose monohydrate to form a composition comprising niraparib and lactose monohydrate;
(c) blending the composition comprising niraparib and lactose monohydrate;
(d) screening the blended composition comprising niraparib and lactose monohydrate;
(e) combining the screened composition comprising niraparib and lactose monohydrate with magnesium stearate to form a composition comprising niraparib, lactose monohydrate and magnesium stearate; and
(f) blending the composition comprising niraparib, lactose monohydrate and magnesium stearate.

33. The method of claim 32, wherein the blended composition comprising niraparib and lactose monohydrate is screened with a screen having a mesh size of about 600 microns.

34. The method of claim 32 or 33, wherein the lactose monohydrate has been screened before combining the screened niraparib with the lactose monohydrate to form a composition comprising niraparib and lactose monohydrate.

35. The method of claim 34, wherein the lactose monohydrate has been screened with a screen having a mesh size of at most about 600 microns.

36. The method of claim 34 or 35, wherein over 50% of the screened lactose monohydrate is present as particles with a diameter of between about 53 microns and 500 microns.

37. The method of any one of claims 32-36, wherein obtaining niraparib that has been screened comprises obtaining niraparib that has been screened with a screen having a mesh size of greater than about 425 microns.

38. The method of claim 37, wherein obtaining niraparib that has been screened with a screen having a mesh size of greater than about 425 microns comprises obtaining niraparib that has been screened with a screen having a mesh size of about 850 microns or about 1180 microns.

39. The method of any one of claims 32-38, wherein the magnesium stearate is magnesium stearate screened with a screen having a mesh size of greater than about 250 microns.

40. The method of claim 39, wherein the magnesium stearate is magnesium stearate screened with a screen having a mesh size of about 600 microns.

41. The method of any one of claims 1-40, wherein the screened niraparib has been annealed one or more times.

42. A method of making a formulation comprising niraparib comprising:
(a) obtaining niraparib, wherein optionally niraparib is niraparib that has been screened, wherein the niraparib has been annealed two or more times;
(b) combining the niraparib with lactose monohydrate to form a composition comprising niraparib and lactose monohydrate;
(c) blending the composition comprising niraparib and lactose monohydrate;
(d) combining the blended composition comprising niraparib and lactose monohydrate with magnesium stearate to form a composition comprising niraparib, lactose monohydrate and magnesium stearate; and
(e) blending the composition comprising niraparib, lactose monohydrate and magnesium stearate.

43. The method of claim 42, wherein the blended composition comprising niraparib and lactose monohydrate is screened with a screen having a mesh size of about 600 microns.

44. The method of claim 42 or 43, wherein the lactose monohydrate has been screened before combining the screened niraparib with the lactose monohydrate to form a composition comprising niraparib and lactose monohydrate.

45. The method of claim 44, wherein the lactose monohydrate has been screened with a screen having a mesh size of at most about 600 microns.

46. The method of claim 44 or 45, wherein over 50% of the screened lactose monohydrate is present as particles with a diameter of between about 53 microns and 500 microns.

47. The method of any one of claims 42-46, wherein obtaining niraparib that has been screened comprises obtaining niraparib that has been screened with a screen having a mesh size of greater than about 425 microns.

48. The method of claim 47, wherein obtaining niraparib that has been screened with a screen having a mesh size of greater than about 425 microns comprises obtaining niraparib that has been screened with a screen having a mesh size of about 850 microns or about 1180 microns.

49. The method of any one of claims 42-48, wherein the magnesium stearate is magnesium stearate screened with a screen having a mesh size of greater than about 250 microns.

50. The method of claim 49, wherein the magnesium stearate is magnesium stearate screened with a screen having a mesh size of about 600 microns.

51. The method of any one of claims 42-50, wherein the method further comprises screening the blended composition comprising niraparib and lactose monohydrate before combining the blended composition comprising niraparib and lactose monohydrate with magnesium stearate.

52. The method of claim 51, wherein the blended composition comprising niraparib and lactose monohydrate is screened with a screen having a mesh size of about 600 microns.

53. A method of making a formulation comprising niraparib comprising:
(a) obtaining niraparib that has been screened with a screen having a mesh size of greater than about 425 microns;
(b) obtaining lactose monohydrate that has been screened with a screen;
(c) combining the screened niraparib with lactose monohydrate to form a composition comprising niraparib and lactose monohydrate;
(d) blending the composition comprising niraparib and lactose monohydrate;
(e) screening the blended composition comprising niraparib and lactose monohydrate;
(f) combining the screened composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than about 250 microns; and
(g) blending the composition comprising niraparib, lactose monohydrate and magnesium stearate.

54. The method of claim 53, wherein the niraparib has been annealed one or more times.

55. The method of any one of claims 1-54, wherein the niraparib has been milled.

56. The method of claim 55, wherein the niraparib has been wet milled.

57. The method of any one of claims 1-56, wherein the niraparib is screened, wherein the screening may be delumping or other such powder handling manually or mechanically.

58. The method of any one of claims 1-57, wherein the method further comprises encapsulating the blended the composition comprising niraparib, lactose monohydrate and magnesium stearate into one or more capsules.

59. The method of claim 58, wherein the one or more capsules are gelatin capsules.

60. The method of claim 58 or 59, wherein the encapsulating comprises using an encapsulator.

61. The method of any one of claims 58-60, wherein the encapsulating comprises encapsulating at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 100,000, 150,000, 200,000, 300,000, 400,000, or 500,000 of the one or more capsules.

62. The method of any one of claims 58-61, wherein the encapsulating comprises encapsulating at a rate of at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 75,000, 100,000, 150,000 or 200,000 of the one or more capsules/hour.

63. The method of any one of claims 58-62, wherein the encapsulating comprises encapsulating the one or more capsules from a batch comprising the composition comprising niraparib, lactose monohydrate and magnesium stearate that is in the encapsulator.

64. The method of claim 63, wherein a portion of the volume of the batch in the encapsulator is used to encapsulate the one or more capsules.

65. The method of claim 64, the portion of the volume of the batch in the encapsulator used to encapsulate the one or more capsules is less than about 100%, 99%, 98%, 97%, 96%, 95%, 90%, 85%, 80%, or 75% of a total initial volume of the batch.

66. The method of any one of claims 58-65, wherein one or more parts of the encapsulator are coated with a coating.

67. The method of claim 66, wherein the one or more coated parts comprises a tamping pin, a dosing disc, or both.

68. The method of claim 66 or 67, wherein the coating comprises nickel, chrome, or a combination thereof.

69. The method of anyone of claims 58-68, wherein the encapsulating comprises automatic encapsulation.

70. The method of any one of claims 58-69, wherein adherence of the composition to one or more encapsulating components is reduced or prevented.

71. The method of any one of claims 58-70, wherein jamming of the encapsulator is reduced or prevented.

72. The method of any one of claims 1-71, wherein blending the composition comprising niraparib and lactose monohydrate comprises blending for about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

73. The method of any one of claims 1-72, wherein blending the composition comprising niraparib, lactose monohydrate and magnesium stearate comprises blending for about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

74. The method of any one of claims 1-73, wherein the blending comprises using a blender, and wherein the niraparib is distributed with substantial uniformity throughout the blender.

75. The method of any one of claims 58-74, wherein a dose-to-dose niraparib concentration variation in the one or more capsules is less than about 50%.

76. The method of claim 75, wherein the dose-to-dose niraparib concentration variation in the one or more capsules is less than about 40%.

77. The method of claim 75, wherein the dose-to-dose niraparib concentration variation in the one or more capsules is less than about 30%.

78. The method of claim 75, wherein the dose-to-dose niraparib concentration variation in the one or more capsules is less than about 20%.

79. The method of claim 75, wherein the dose-to-dose niraparib concentration variation in the one or more capsules is less than about 10%.

80. The method of claim 75, wherein the dose-to-dose niraparib concentration variation in the one or more capsules is less than about 5%.

81. The method of any one of claims 75-80, wherein the dose-to-dose niraparib concentration variation is based on 10 consecutive doses or fewer.

82. The method of claim 81, wherein the dose-to-dose niraparib concentration variation is based on 8 consecutive doses.

83. The method of claim 81, wherein the dose-to-dose niraparib concentration variation is based on 5 consecutive doses.

84. The method of claim 81, wherein the dose-to-dose niraparib concentration variation is based on 3 consecutive doses.

85. The method of claim 81, wherein the dose-to-dose niraparib concentration variation is based on 2 consecutive doses.

86. A formulation comprising
(a) an effective amount of niraparib to inhibit polyadenosine diphosphate ribose polymerase (PARP) when administered to a human,
(b) lactose monohydrate, and
(c) magnesium stearate;
wherein the formulation comprising niraparib, lactose monohydrate and magnesium stearate produced according the method of any one of claims 1-85.

87. A formulation comprising
(a) an effective amount of niraparib to inhibit polyadenosine diphosphate ribose polymerase (PARP) when administered to a human,
(b) lactose monohydrate, and
(c) magnesium stearate.

88. A formulation comprising
(a) an effective amount of niraparib to inhibit polyadenosine diphosphate ribose polymerase (PARP) when administered to a human,
(b) lactose monohydrate, and
(c) magnesium stearate;
wherein the niraparib has been annealed two or more times.

89. A formulation comprising
(a) an effective amount of niraparib to inhibit polyadenosine diphosphate ribose polymerase (PARP) when administered to a human,
(b) lactose monohydrate, and
(c) magnesium stearate;
wherein the niraparib in the capsule has a Hausner's ratio of less than about 1.7.

90. The formulation of claim 89, wherein the niraparib has a Hausner's ratio of about 1.48 or less.

91. The formulation of claim 89, wherein the niraparib has a Hausner's ratio of about 1.38 or less.

92. A formulation comprising
(a) an effective amount of niraparib to inhibit polyadenosine diphosphate ribose polymerase (PARP) when administered to a human,
(b) lactose monohydrate, and
(c) magnesium stearate;
wherein the formulation has a Hausner's ratio of about 1.7 or less.

93. The formulation of claim 92, wherein the formulation has a Hausner's ratio of about 1.64 or less.

94. The formulation of claim 92, wherein the formulation has a Hausner's ratio of about 1.52 or less.

95. The formulation of claim 92, wherein the formulation has a Hausner's ratio of about 1.47 or less.

96. The formulation of claim 92, wherein the formulation has a Hausner's ratio of about 1.43 or less.

97. The formulation of claim 92, wherein the formulation has a Hausner's ratio of about 1.41 or less.

98. A formulation comprising
(a) an effective amount of niraparib to inhibit polyadenosine diphosphate ribose polymerase (PARP) when administered to a human,
(b) lactose monohydrate, and
(c) magnesium stearate;
wherein the lactose monohydrate has (i) a bulk density of about 0.2-0.8 mg/cm³ and/or (ii) a tapped density of about 0.3-0.9 mg/cm³.

99. A formulation comprising
(a) an effective amount of niraparib to inhibit polyadenosine diphosphate ribose polymerase (PARP) when administered to a human,
(b) lactose monohydrate particles, and
(c) magnesium stearate;
wherein about 50% or more of the lactose monohydrate particles has a diameter of at least about 53 microns to about 500 microns, and/or about 50% or more of the lactose monohydrate particles has a diameter of at most about 250 microns.

100. The formulation of any one of claims 86-99, wherein the niraparib has an internal friction angle of about 33.1 degrees or higher.

101. The formulation of any one of claims 86-100, wherein the formulation has an internal friction angle of less than about 34 degrees.

102. The formulation of any one of claims 86-101, wherein the niraparib has a flow function ratio value of more than about 6.4.

103. The formulation of any one of claims 86-102, wherein the formulation has a flow function ratio value of more than about 14.4.

104. The formulation of any one of claims 86-103, wherein the niraparib has a wall friction angle of less than about 29 at an Ra of about 0.05.

105. The formulation of any one of claims 86-104, wherein the formulation has a wall friction angle of less than about 15 degrees at an Ra of about 0.05.

106. The formulation of any one of claims 86-105, wherein the formulation has a wall friction angle of less than about 26 degrees at an Ra of about 1.2.

107. The formulation of any one of claims 86-106, wherein the formulation is stable with respect to niraparib degradation after storage for about 1 month, 3 months, 6 months, 9 months, 12 months, 24 months, or 36 months.

108. The formulation of claim 107, wherein the formulation is stable with respect to niraparib degradation after storage for about 1 month, 3 months, 6 months, 9 months, 12 months, 24 months, or 36 months at 5° C.

109. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 5° C.

110. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 25° C. and about 60% relative humidity (RH).

111. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 30° C. and about 65% relative humidity (RH).

112. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for about 1 month, 3 months, 6 months, 9 months, 12 months, 24 months, or 36 months at about 40° C. and about 75% relative humidity (RH)

113. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 5° C.

114. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 25° C. and about 60% relative humidity (RH).

115. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 30° C. and about 65% relative humidity (RH).

116. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for about 1 month, 3 months, 6 months, 9 months, 12 months, 24 months, or 36 months at about 40° C. and about 75% relative humidity (RH).

117. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 5° C.

118. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 25° C. and about 60% relative humidity (RH).

119. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for 1 month, 3 months, 6 months, 9 months, 12 months, 24 months, or 36 months at about 30° C. and about 65% relative humidity (RH).

120. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for about 1 month, 3 months, 6 months, 9 months, 12 months, 24 months, or 36 months at about 40° C. and about 75% relative humidity (RH).

121. The formulation of claim 107, wherein the formulation comprises less than about 3.0%, 2.5%, 2.0%, 1.5% 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 5° C.

122. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 30° C. and about 65% relative humidity (RH).

123. The formulation of claim 107, wherein the formulation comprises less than about 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for about 1 month, 3 months, 6 months, 9 months, 12 months, 24 months, or 36 months at about 40° C. and about 70% relative humidity (RH).

124. The formulation of any one of claims 86-123, wherein the formulation has an absolute bioavailability of niraparib of about 60 to about 90%.

125. The formulation of any one of claims 86-124, wherein not less than about 30%, 35%, 40%, 45%, 55%, 60%, 65% 70%, 75%, 80%, 85%, 90%, 95%, or 100% of the niraparib dissolves in about 5, 10, 15, 20, 30, 45, 60, 90, or 120 minutes under dissolution evaluation.

126. The formulation of claim 125 or 126, wherein not less than about 30%, 35%, 40%, 45%, 55%, 60%, 65% 70%, 75%, 80%, 85%, 90%, 95%, or 100% of the niraparib dissolves in about 5, 10, 15, 20, 30, 45, 60, 90, or 120 minutes under dissolution evaluation after storage of the composition for about 1 month, 3 months, 6 months, 9 months, 12 months, 24 months, or 36 months at about 25° C. and about 60% relative humidity (RH).

127. The formulation of any one of claims 86-126, comprising niraparib tosylate monohydrate in an amount that is about 19.16%, 38.32%, 57.48%, or 76.64% by weight of the composition.

128. The formulation of any one of claims 86-126, comprising niraparib tosylate monohydrate in an amount that is about 19.2 to about 38.3% w/w niraparib.

129. The formulation of any one of claims 86-126, comprising about 50 mg to about 300 mg of niraparib tosylate monohydrate, about 100 mg to about 200 mg of niraparib tosylate monohydrate, or about 125 mg to about 175 mg of niraparib tosylate monohydrate.

130. The formulation of claim 129, comprising about 79.7 mg, about 159.4 mg, about 318.8 mg, or about 478.2 mg niraparib tosylate monohydrate.

131. The formulation of any one of claims 86-126, comprising about 100 mg of niraparib based on free base.

132. The formulation of claim 131, comprising about 159.4 mg niraparib tosylate monohydrate.

133. The formulation of any one of claims 86-132, comprising about 61.2 to about 80.3% w/w lactose monohydrate.

134. The formulation of any one of claims 86-133, comprising at least about 0.5% w/w magnesium stearate.

135. A capsule comprising the formulation of any one of claims 86-134.

136. A method of treating cancer, comprising administering to a subject in need thereof the formulation according to any one of claims 86-134 or the capsule of claim 135.

137. The method of claim 136, wherein the capsule is administered in doses having a dose-to-dose niraparib concentration variation of less than 50%, less than 40%, less than 30%, less than 20%, less than 10%, or less than 5%.

138. The method of claim 136 or 137, wherein the cancer is selected from the group consisting of ovarian cancer, breast cancer, cervical cancer, endometrial cancer, prostate cancer, testicular cancer, pancreatic cancer, esophageal cancer, head and neck cancer, gastric cancer, bladder cancer, lung cancer, bone cancer, colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma, bladder cancer, liver cancer, kidney cancer, myeloma, lymphoma, and combinations thereof.

139. The method of any one of claims 136-138, wherein the cancer is selected from the group consisting of ovarian cancer, fallopian tube cancer, primary peritoneal cancer, and combinations thereof.

140. The method of any one of claims 136-139, wherein the cancer is a recurrent cancer.

141. The method of any one of claims 136-140, wherein the subject is a human subject.

142. The method of claim 141, wherein the human subject was previously treated with a chemotherapy.

143. The method of claim 142, wherein a chemotherapy is a platinum-based chemotherapy.

144. The method of claim 142 or 143, wherein the human subject had a complete or partial response to the chemotherapy.

145. The method of any one of claims 136-144, wherein the subject has a mean peak plasma concentration (Cₘₐₓ) of 600 ng/mL to 1000 ng/mL of the niraparib.

146. The method of claim 145, wherein the subject has the mean peak plasma concentration (Cₘₐₓ) within 0.5 to 6 hours after the administering.

147. The method of any one of claims 136-146, wherein about 60%, 65%, 70%, 75%, 80%, 85% or 90% of the niraparib is bound to human plasma protein of the subject after the administering.

148. The method of any one of claims 136-147, wherein an apparent volume of distribution (Vd/F) of the niraparib is from about 500 L to about 2000 L after administration to a human subject.

149. The method of any one of claims 136-148, wherein the niraparib has a mean terminal half-life (t_{1/2}) of from about 30 to about 60 hours after the administering.

150. The method of any one of claims 136-149, wherein the niraparib has an apparent total clearance (CL/F) of from about 10 L/hour to about 20 L/hour after the administering.

151. The method of any one of claims 136-150, wherein at least about 60%, 65%, 70%, 75%, 80%, 85%, 90%, 95%, 96%, 97%, 98%, 99%, or 100% of the niraparib is released from the composition within 1 minute, or within 5 minutes, or within 10 minutes, or within 15 minutes, or within 30 minutes, or within 60 minutes or within 90 minutes after the administering.

152. The method of any one of claims 136-151, wherein the subject has a Cₘᵢₙ niraparib blood plasma level at steady state of from about 10 ng/ml to about 100 ng/ml after the administering.

153. The method of any one of claims 136-152, wherein at least about 70%, 80%, 90%, or 95% of the niraparib is absorbed into the bloodstream of the subject within 1, 2, 3, 4, 5, 6, 7, 8, 9, 10, 11, 12, 16, 18, or 24 hours after administering.
